SEQUENCE LISTING (4 sequences)

SEQ ID NO: 1 - protein, 23 aa, Artificial Sequence
  Synthetic Polypeptide
MAAVGPRTGPGTGAEALALAAEL

SEQ ID NO: 2 - protein, 13 aa, Artificial Sequence
  Synthetic Polypeptide
AATRAPPFPLPCP

SEQ ID NO: 3 - protein, 16 aa, Artificial Sequence
  Synthetic Polypeptide
HGSQAAAARAAAARCG

SEQ ID NO: 4 - protein, 30 aa, Artificial Sequence
  Synthetic Polypeptide
NVSLKTFVLKGMLKKFKEDLRGELEKEEKV

(19) United States
(12) Patent Application Publication
(10) Pub. No.: US 2024/0011101 A1
(43) Pub. Date: Jan. 11, 2024

(54) METHOD OF DETERMINING RESISTANCE TO CHECKPOINT INHIBITOR THERAPIES

(71) Applicant: Shattuck Labs, Inc., Austin, TX (US)

(72) Inventors: Taylor SCHREIBER, Austin, TX (US); George FROMM, Austin, TX (US); Suresh DE SILVA, Austin, TX (US)

(21) Appl. No.: 18/265,046

(22) PCT Filed: Dec. 3, 2021
(86) PCT No.: PCT/US21/61834

§ 371 Date: Jun. 2, 2023

Related U.S. Application Data
(60) Provisional application No. 63/276,066, filed on Nov. 5, 2021.
(60) Provisional application No. 63/215,735, filed on Jun. 28, 2021.
(60) Provisional application No. 63/173,090, filed on Apr. 9, 2021.
(60) Provisional application No. 63/121,083, filed on Dec. 3, 2020.

(51) Int. Cl. C12Q 1/6886; A01K 67/027; G01N 33/50

(52) U.S. Cl. CPC C12Q 1/6886; A01K 67/0275; G01N 33/5088; C12Q 2600/106; A01K 2227/105; A01K 2267/0331; A01K 2207/12

(57) ABSTRACT

The current disclosure relates to animal models, methods for screening and testing of an anti-cancer drug candidates, and to methods for treatment, evaluating efficacy of cancer treatment, and selecting patients for cancer therapy.

### PRIORITY

This application claims the benefit of, and priority to, U.S. Provisional Application No. 63/121,083, filed Dec. 3, 2020; 63/173,090, filed Apr. 9, 2021; 63/215,735, filed Jun. 28, 2021; and 63/276,066, filed Nov. 5, 2021, the contents of each of which are hereby incorporated by reference in its entirety.

### FIELD OF THE DISCLOSURE

The present disclosure relates to, in part, methods that are useful for detection and treatment of drug resistant cancer, and methods for developing new therapeutics against drug resistant cancers.

### DESCRIPTION OF THE TEXT FILE SUBMITTED ELECTRONICALLY

The contents of the text file submitted electronically herewith are incorporated herein by reference in their entirety: a computer readable format copy of the Sequence Listing (filename: SHK-038PC1_ST25; date created: Dec. 1, 2021; size 1,571 bytes).

### BACKGROUND

Drug resistance remains one of the biggest challenges in cancer therapy. Drug resistance is found across all types of cancer and all modes of treatment, including molecularly targeted therapy, immunotherapy, and chemotherapy. Moreover, the treatment of cancer typically is approached empirically, and many patients with chemo-resistant disease receive multiple cycles of often toxic therapy before the lack of efficacy becomes evident. The inability to predict responses to specific therapies is a major impediment to improving outcome for cancer patients. In some patients, initiation of efficacious therapy is delayed by the inability to predict responses. Moreover, it is also common that a patient with advanced cancer receives a drug that helps shrink their tumors, but then weeks or months later the cancer comes back and the drug no longer works. Unfortunately, few effective therapeutic options are available for some patients having cancers that are resistant to the anti-checkpoint therapies. Thus, drug resistance, either existing before treatment (intrinsic or primary resistance) or developed after therapy (acquired resistance), is responsible for most relapses of cancer, one of the major causes of death. Therefore, better understanding the mechanisms of drug resistance is required to provide guidance to future cancer treatment. Moreover, methods for developing new therapies for patients suffering from drug resistant cancer and methods for selecting appropriate drugs for patients having drug-resistant cancer are required for improving outcomes in cancer patients.

### SUMMARY

Accordingly, the present disclosure provides, in part, methods for selecting patients for cancer treatment, and methods for cancer treatment, based on, for instance, based on gene expression profiles of anti-PD-1 resistant cancers. The present disclosure also provides animal models suitable for testing an anti-cancer drug candidate, and methods for making a pharmaceutical composition for treating cancer.

In one aspect, the present disclosure relates to a method of determining a cancer treatment for a patient, the method comprising: (a) obtaining a biological sample from a subject; (b) evaluating the sample for the presence, absence, or level of one or more gene associated with a gene ontology (GO) pathway selected from: (i) positive regulation of cell cycle process, regulation of G1/S transition, regulation of cell division, regulation of cell proliferation, positive regulation of IκB kinase/NFκB signaling, type I IFN signaling pathway, cellular response to IFNγ, positive regulation of IFNα production, positive regulation of defense response, positive regulation of IFNβ production, regulation of inflammatory response, regulation of innate immune response, negative regulation of antigen processing/presentation, and antigen processing/presentation of endogenous peptides via MHC class I; and/or (ii) phospholipid efflux, negative regulation of fibrinolysis, chylomicron assembly, plasma membrane repair, SRP-dependent co-translational protein targeting to membrane, ribosomal small subunit assembly, phospholipid efflux, regulation of translation, mitochondrial respiratory chain complex I, mitochondrial translational elongation, DNA-dependent DNA replication, and ATP biosynthetic process; and (c) selecting the cancer therapy with an ability to inhibit function and/or activity of PD-1, PD-L1 and/or PD-L2 based on the evaluation of step (b). In embodiments, the mouse belongs to BALB/c or C57BL/6 strain. In embodiments, the cancer therapy that has the ability inhibit function and/or activity of PD-1, PD-L1 and/or PD-L2 is an antibody. In embodiments, the antibody is selected from nivolumab (OPDIVO), pembrolizumab (KEYTRUDA), pidilizumab (CT-611, CURE TECH), MK-3475 (MERCK), BMS 936559, MPDL328OA (ROCHE), Cemiplimab (LIBTAYO), Atezolizumab (TECENTRIQ), Avelumab (BAVENCIO), and Durvalumab (imfinzi).

In one aspect, the present disclosure relates to a method for selecting a patient for a cancer treatment, the method comprising: (a) obtaining a biological sample from a subject; (b) evaluating the sample for the presence, absence, or level of one or more genes associated with a gene ontology (GO) pathway selected from: (i) positive regulation of cell cycle process, regulation of G1/S transition, regulation of cell division, regulation of cell proliferation, positive regulation of IκB kinase/NFκB signaling, type I IFN signaling pathway, cellular response to IFNγ, positive regulation of IFNα production, positive regulation of defense response, positive regulation of IFNβ production, regulation of inflammatory response, regulation of innate immune response, negative regulation of antigen processing/presentation, and antigen processing/presentation of endogenous peptides via MHC class I; and/or (ii) phospholipid efflux, negative regulation of fibrinolysis, chylomicron assembly, plasma membrane repair, SRP-dependent co-translational protein targeting to membrane, ribosomal small subunit assembly, phospholipid efflux, regulation of translation, mitochondrial respiratory chain complex I, mitochondrial translational elongation, DNA-dependent DNA replication, and ATP biosynthetic process; and (c) selecting a cancer therapy with an ability to inhibit function and/or activity of PD-1, PD-L1 and/or PD-L2. In embodiments, the mouse belongs to BALB/c or C57BL/6 strain. In embodiments, the cancer therapy that has the ability inhibit function and/or activity of PD-1, PD-L1 and/or PD-L2 is an antibody. In embodiments, the antibody is selected from nivolumab (OPDIVO), pembrolizumab (KEYTRUDA), pidilizumab (CT-011, CURE TECH), MK-3475 (MERCK), BMS 936559, MPDL328OA (ROCHE), Cemiplimab (LIBTAYO), Atezolizumab (TECENTRIQ), Avelumab (BAVENCIO), and Durvalumab (imfinzi).

In one aspect, the present disclosure relates to a method of treating cancer, the method comprising: (a) obtaining a biological sample from a subject; (b) evaluating the sample for the presence, absence, or level of one or more genes associated with a gene ontology (GO) pathway selected from: (i) positive regulation of cell cycle process, regulation of G1/S transition, regulation of cell division, regulation of cell proliferation, positive regulation of 1 KB kinase/NFκB signaling, type I IFN signaling pathway, cellular response to IFNγ, positive regulation of IFNα production, positive regulation of defense response, positive regulation of IFNβ production, regulation of inflammatory response, regulation of innate immune response, negative regulation of antigen processing/presentation, and antigen processing/presentation of endogenous peptides via MHC class I; and/or (ii) phospholipid efflux, negative regulation of fibrinolysis, chylomicron assembly, plasma membrane repair, SRP-dependent co-translational protein targeting to membrane, ribosomal small subunit assembly, phospholipid efflux, regulation of translation, mitochondrial respiratory chain complex I, mitochondrial translational elongation, DNA-dependent DNA replication, and ATP biosynthetic process; and (c) selecting the cancer therapy selected from: (i) an agent with an ability to inhibit function and/or activity of PD-1, PD-L1 and/or PD-L2; (ii) an antimetabolite chemotherapeutic (e.g., 5-fluorouracil, methotrexate, capecitabine, azacitidine, 6-diazo-5-oxo-L-norleucine (DON), azaserine and acivicin), a topoisomerase inhibitor (e.g., irinotecan, topotecan, etoposide, doxorubicin, etc.); and (iii) a protein translation inhibitor (e.g., silvestrol and omacetaxine) ribosome biogenesis inhibitors (e.g., diazaborine, lamotrigine and ribozinoindoles), inhibitors of rRNA and/or tRNA synthesis (e.g., quarfloxin (CX-3543) and CX-5461), an inhibitor of synthesis of amino acids (e.g., GLUD1 inhibitor R162, BCAT1 inhibitor gabapentin, glutaminase inhibitor bis-2-(5-phenylacetamido-1,2,4-thiadiazol-2-yl)ethyl sulfide (BPTES), PAGDH inhibitor NCT-503), an inhibitor of uptake of amino acids (e.g., SLC7A11 inhibitors sulfasalazine, erastin or sorafenib), a modulator of post-translational modification (e.g., glycosylation inhibitor tunicamycin, ppGalNAc-T3), a modulator of protein degradation, and a modulator of protein transport (e.g., cyclosporin A, fendiline, parbendazole, paroxetine, parthenolide, quinacrine, sertraline, spiperone, thimerosal, astemizole, perhexiline, HUN-7293, CAM741, CK147, and cotransin); and (d) administering the cancer therapy with an ability to inhibit function and/or activity of PD-1, PD-L1 and/or PD-L2; and optionally administering the therapy selected in step (c)(ii) and (c)(iii). In embodiments, the mouse belongs to BALB/c or C57BL/6 strain. In embodiments, the cancer therapy that has the ability inhibit function and/or activity of PD-1, PD-L1 and/or PD-L2 is an antibody. In embodiments, the antibody is selected from nivoiumab (OPDIVO), pembrolizumab (KEYTRUDA), pidilizumab (CT-011, CURE TECH), MK-3475 (MERCK), BMS 936559, MPDL3280A (ROCHE), Cemiplimab (LIBTAYO), Atezolizumab (TECENTRIQ), Avelumab (BAVENCIO), and Durvalumab (imfinzi).

In embodiments, a lack of upregulation of one or more genes associated with cellular response to IFNγ compared to a prior biological sample from the subject indicates a response to the cancer therapy with an ability to inhibit function and/or activity of PD-1, PD-L1 and/or PD-L2. In embodiments, a lack of upregulation of one or more genes associated with cellular response to IFNγ compared to a patient that is known to be sensitive to anti-PD-1 therapy indicates a response to the cancer therapy with an ability to inhibit function and/or activity of PD-1, PD-L1 and/or PD-L2. In embodiments, a lack of upregulation of one or more genes associated with cellular response to IFNγ compared to a standard indicates a response to the cancer therapy with an ability to inhibit function and/or activity of PD-1, PD-L1 and/or PD-L2.

In embodiments, an upregulation of one or more genes associated with cellular response to IFNγ compared to a prior biological sample from the subject indicates resistance, a lack of response, or recalcitrance to the cancer therapy with an ability to inhibit function and/or activity of PD-1, PD-L1 and/or PD-L2. In embodiments, an upregulation of one or more genes associated with cellular response to IFNγ compared to a patient that is known to be sensitive to anti-PD-1 therapy indicates resistance, a lack of response, or recalcitrance to the cancer therapy with an ability to inhibit function and/or activity of PD-1, PD-L1 and/or PD-L2. In embodiments, an upregulation of one or more genes associated with cellular response to IFNγ compared to a standard indicates resistance, a lack of response, or recalcitrance to the cancer therapy with an ability to inhibit function and/or activity of PD-1, PD-L1 and/or PD-L2.

In embodiments, an upregulation of one or more genes associated with type I IFN signaling pathway compared to a prior biological sample from the subject indicates resistance, a lack of response, or recalcitrance to the cancer therapy with an ability to inhibit function and/or activity of PD-1, PD-L1 and/or PD-L2. In embodiments, an upregulation of one or more genes associated with type I IFN signaling pathway compared to a patient that is known to be sensitive to anti-PD-1 therapy indicates resistance, a lack of response, or recalcitrance to the cancer therapy with an ability to inhibit function and/or activity of PD-1, PD-L1 and/or PD-L2. In embodiments, an upregulation of one or more genes associated with type I IFN signaling pathway compared to a standard indicates resistance, a lack of response, or recalcitrance to the cancer therapy with an ability to inhibit function and/or activity of PD-1, PD-L1 and/or PD-L2.

In embodiments, a lack of upregulation of one or more genes associated with type I IFN signaling pathway compared to a prior biological sample from the subject indicates a response to the cancer therapy with an ability to inhibit function and/or activity of PD-1, PD-L1 and/or PD-L2. In embodiments, a lack of upregulation of one or more genes associated with type I IFN signaling pathway compared to a patient that is known to be sensitive to anti-PD-1 therapy indicates a response to the cancer therapy with an ability to inhibit function and/or activity of PD-1, PD-L1 and/or PD-L2. In embodiments, a lack of upregulation of one or more genes associated with type I IFN signaling pathway compared to a standard indicates a response to the cancer therapy with an ability to inhibit function and/or activity of PD-1, PD-L1 and/or PD-L2.

In embodiments, the biological sample is a fresh tissue sample, frozen tumor tissue specimen, cultured cells, circulating tumor cells, or a formalin-fixed paraffin-embedded tumor tissue specimen. In embodiments, the biological sample is a biopsy sample. In embodiments, the biological sample comprises a body fluid selected from blood, plasma, serum, lacrimal fluid, tears, bone marrow, blood, blood cells, ascites, tissue or fine needle biopsy sample, cell-containing body fluid, free floating nucleic acids, sputum, saliva, urine, cerebrospinal fluid, peritoneal fluid, pleural fluid, feces, lymph, gynecological fluid, skin swab, vaginal swab, oral swab, nasal swab, washing or lavage such as a ductal lavage or broncheoalveolar lavage, aspirate, scraping, bone marrow specimen, tissue biopsy specimen, surgical specimen, feces, other body fluids, secretions, and/or excretions, and/or cells therefrom.

In embodiments, the biological sample comprises at least one tumor cell.

In embodiments, the evaluating is performed by DNA sequencing, RNA sequencing, immunohistochemical staining, western blotting, in cell western, immunofluorescent staining, ELISA, and fluorescent activating cell sorting (FACS) or a combination thereof.

In embodiments, the evaluating is performed by contacting the sample with an agent that specifically binds to one or more proteins encoded by one or more genes associated with a gene ontology (GO) pathways identified herein.

In embodiments, the evaluating is performed by contacting the sample with an agent that specifically binds to one or more of nucleic acids of one or more genes associated with a gene ontology (GO) pathway identified herein.

In embodiments, the evaluating informs classifying the patient into a high or low risk group. In embodiments, the high risk classification comprises a high level of tumor cells having resistance to the cancer therapy with an ability to inhibit function and/or activity of Pa-1, PD-L1 and/or PD-L2, In embodiments, the low risk classification comprises a low level of tumor cells having resistance to the cancer therapy with an ability to inhibit function and/or activity of PD-1, PD-L1 and/or PD-L2.

In one aspect, the present disclosure relates to a transgenic non-human animal comprising one or more tumor cells, wherein the tumor cells have: (a) upregulation of one or more genes associated with a gene ontology (GO) pathway selected from: positive regulation of cell cycle process, regulation of &I'S transition, regulation of cell division, regulation of cell proliferation, positive regulation of IκB kinase/NFκB signaling, type I IFN signaling pathway, cellular response to IFNγ, positive regulation of IFNα production, positive regulation of defense response, positive regulation of IFNβ production, regulation of inflammatory response, regulation of innate immune response, negative regulation of antigen processing and presentation, and antigen processing, presentation of endogenous peptides via MHC class I; and/or (b) downregulation of one or more genes associated with a gene ontology (GO) pathway selected from: phospholipid efflux, negative regulation of fibrinolysis, chylomicron assembly, plasma membrane repair, SRP-dependent co-translational protein targeting to membrane, ribosomal small subunit assembly, phospholipid efflux, regulation of translation, mitochondrial respiratory chain complex I, mitochondrial translational elongation, DNA-dependent DNA replication, and ATP biosynthetic process. In embodiments, the transgenic non-human animal is transgenic. In embodiments, the tumor cells are resistant to a cancer therapy with an ability to inhibit function and/or activity of PD-1, PD-L1 and/or PD-L2. In embodiments, the transgenic non-human animal is a rodent. In embodiments, the rodent is a mouse. In embodiments, the mouse belongs to BALB/c or C57BL/6 strain.

In one aspect, the present disclosure relates to a method for testing an anti-cancer drug candidate, the method comprising: (a) providing a transgenic non-human animal of any one of any of the embodiments disclosed herein; (b) administering the anti-cancer drug candidate to the transgenic non-human animal, and (c) evaluating whether the anti-cancer drug candidate is effective in slowing or inhibiting cancer growth in the transgenic non-human animal. In one aspect, the present disclosure relates to the anti-cancer drug candidate is selected from a chemotherapeutic agent, a cytotoxic agent, and a checkpoint inhibitor.

In one aspect, the present disclosure relates to a method for making a pharmaceutical composition for treating cancer, the method comprising: (a) providing a transgenic non-human animal of any one of any of the embodiments disclosed herein; (b) administering the anti-cancer drug candidate to the transgenic non-human animal, and (c) selecting an anti-cancer drug that is effective in slowing or inhibiting cancer growth in the transgenic non-human animal; and (d) formulating the anti-cancer drug or candidate for administration to a human patient. In one aspect, the present disclosure relates to the anti-cancer drug candidate is selected from a chemotherapeutic agent, a cytotoxic agent, and a checkpoint inhibitor.

In embodiments, the upregulation is in comparison to a healthy tissue. In embodiments, the upregulation is in comparison to another biological sample from patient that is known to be sensitive to anti-PD-1 therapy. In embodiments, the upregulation is in comparison to a prior biological sample obtained from the subject. In embodiments, the downregulation is in comparison to a healthy tissue. In embodiments, the downregulation is in comparison to another biological sample from patient that is known to be sensitive to anti-PD-1 therapy. In embodiments, the downregulation is in comparison to a prior biological sample obtained from the subject.

In embodiments, the anti-checkpoint agent an antibody selected from nivolumab (OPDIVO), pembrolizumab (KEYTRUDA), pidilizumab (CT-011, CURE TECH), MK-3475 (MERCK), BMS 936559, MPDL3280A (ROCHE), Cemiplimab (LIBTAYO), Atezolizumab (TECENTRIQ), Avelumab (BAVENCIO), and Durvalumab (imfinzi).

In one aspect, the present disclosure relates to a method of determining a cancer treatment for a patient, the method comprising: (a) obtaining a biological sample from a subject; (b) evaluating the biological sample for the expression of: (i) a gene selected from CD274, B2M, STAT1, STAT2, TRIM7, IRF1, TAP1, TAP2, CASP1, IRF, LTBR, PVR, GASTA3, LRG1, SPRY2, ARG1, TRIM8, TRIM2, MAPK8IP1, TRIM6, and KRT1; and/or (ii) a gene selected from RPL41, RPS15, RPS8, TRIM7 and LRG1; and (c) selecting the cancer therapy based on the evaluation of step (b), wherein cancer therapy is selected from: (i) an agent with an ability to inhibit function and/or activity of PD-1, PD-L1 and/or PD-L2; (ii) an antimetabolite chemotherapeutic (e.g., 5-fluorouracil, methotrexate, capecitabine, azacitidine, 6-diazo-5-oxo-L-norleucine (DON), azaserine and acivicin), a topoisomerase inhibitor (e.g., irinotecan, topotecan, etoposide, doxorubicin, etc.); and (iii) a protein translation inhibitor (e.g., silvestrol and omacetaxine) ribosome biogenesis inhibitors (e.g., diazaborine, lamotrigine and ribozinoindoles), inhibitors of rRNA and/or tRNA synthesis (e.g., quarfloxin (CX-3543) and CX-5461), an inhibitor of synthesis of amino acids (e.g., GLUD1 inhibitor R162, BCAT1 inhibitor gabapentin, glutaminase inhibitor bis-2-(5-phenylacetamido-1,2,4-thiadiazol-2-yl)ethyl sulfide (BPTES), PAGDH inhibitor NCT-503), an inhibitor of uptake of amino acids (e.g., SLC7A11 inhibitors sulfasalazine, erastin or sorafenib), a modulator of post-translational modification (e.g., glycosylation inhibitor tunicamycin, ppGalNAc-T3), a modulator of protein degradation, and a modulator of protein transport (e.g., cyclosporin A, fendiline, parbendazole, paroxetine, parthenolide, quinacrine, sertraline, spiperone, thimerosal, astemizole, perhexiline, HUN-7293, CAM741, CK147, and cotransin, etc.) or a topoisomerase inhibitor.

In one aspect, the present disclosure relates to a method for selecting a patient for a cancer treatment, the method comprising: (a) obtaining a biological sample from a subject; (b) evaluating the biological sample for the expression of: (i) a gene selected from CD274, B2M, STAT1, STAT2, TRIM7, IRF1, TAP1, TAP2, CASP1, IRF, LTBR, PVR, GASTA3, LRG1, SPRY2, ARG1, TRIM8, TRIM2, MAPK81P1, TRIM6, and KRT1; and/or (ii) a gene selected from RPL41, RPS15, RPS8, TRIM7 and LRG1; and (c) selecting the cancer therapy based on the evaluation of step (b), wherein cancer therapy is selected from: (i) an agent with an ability to inhibit function and/or activity of PD-1, PD-L1 and/or PD-L2; (ii) an antimetabolite chemotherapeutic (e.g., 5-fluorouracil, methotrexate, capecitabine, azacitidine, 6-diazo-5-oxo-L-norleucine (DON), azaserine and acivicin), a topoisomerase inhibitor (e.g., irinotecan, topotecan, etoposide, doxorubicin, etc.); and (iii) a protein translation inhibitor (e.g., silvestrol and omacetaxine) ribosome biogenesis inhibitors (e.g., diazaborine, lamotrigine and ribozinoindoles), inhibitors of rRNA and/or tRNA synthesis (e.g., quarfloxin (CX-3543) and CX-5461), an inhibitor of synthesis of amino acids (e.g., GLUD1 inhibitor R162, BCAT1 inhibitor gabapentin, glutaminase inhibitor bis-2-(5-phenylacetamido-1,2,4-thiadiazol-2-yl)ethyl sulfide (BPTES), PAGDH inhibitor NCT-503), an inhibitor of uptake of amino acids (e.g., SLC7A11 inhibitors sulfasalazine, erastin or sorafenib), a modulator of post-translational modification (e.g., glycosylation inhibitor tunicamycin, ppGalNAc-T3), a modulator of protein degradation, and a modulator of protein transport (e.g., cyclosporin A, fendiline, parbendazole, paroxetine, parthenolide, quinacrine, sertraline, spiperone, thimerosal, astemizole, perhexiline, HUN-7293, CAM741, CK147, and cotransin, etc.) or a topoisomerase inhibitor. In one aspect, the present disclosure relates to a method of treating cancer, the method comprising: (a) obtaining a biological sample from a subject; (b) evaluating the biological sample for the expression of: (i) a gene selected from CD274, B2M, STAT1, STAT2, TRIM7, IRF1, TAP1, TAP2, CASP1, IRF, LTBR, PVR, GASTA3, LRG1, SPRY2, ARG1, TRIM8, TRIM2, MAPK81P1, TRIM6, and KRT1; and/or (ii) a gene selected from RPL41, RPS15, RPS8, TRIM7 and LRG1; and (c) selecting the cancer therapy selected from: (i) an agent with an ability to inhibit function and/or activity of PD-1, PD-L1 and/or PD-L2; (ii) an antimetabolite chemotherapeutic (e.g., 5-fluorouracil, methotrexate, capecitabine, azacitidine, 6-diazo-5-oxo-L-norleucine (DON), azaserine and acivicin), a topoisomerase inhibitor (e.g., irinotecan, topotecan, etoposide, doxorubicin, etc.); and (iii) a protein translation inhibitor (e.g., silvestrol and omacetaxine) ribosome biogenesis inhibitors (e.g., diazaborine, lamotrigine and ribozinoindoles), inhibitors of rRNA and/or tRNA synthesis (e.g., quarfloxin (CX-3543) and CX-5461), an inhibitor of synthesis of amino acids (e.g., GLUD1 inhibitor R162, BCAT1 inhibitor gabapentin, glutaminase inhibitor bis-2-(5-phenylacetamido-1,2,4-thiadiazol-2-yl)ethyl sulfide (BPTES), PAGDH inhibitor NCT-503), an inhibitor of uptake of amino acids (e.g., SLC7A11 inhibitors sulfasalazine, erastin or sorafenib), a modulator of post-translational modification (e.g., glycosylation inhibitor tunicamycin, ppGalNAc-T3), a modulator of protein degradation, and a modulator of protein transport (e.g., cyclosporin A, fendiline, parbendazole, paroxetine, parthenolide, quinacrine, sertraline, spiperone, thimerosal, astemizole, perhexiline, HUN-7293, CAM741, CK147, and cotransin, etc.) or a topoisomerase inhibitor; and (d) administering the cancer therapy with an ability to inhibit function and/or activity of PD-1, PD-L1 and/or PD-L2; and optionally administering the therapy selected in step (c)(ii) and (c)(iii).

In one aspect, the present disclosure relates to a method of determining a cancer treatment for a patient, the method comprising: (a) obtaining a biological sample from a subject; (b) evaluating the biological sample for the activation of a pathway selected from Mapk8ip1, Trim7, Elk1, Lrg1, Arg1, Rap1, and Ras; and (c) selecting the cancer therapy based on the evaluation of step (b), wherein cancer therapy is selected from: (i) an agent with an ability to inhibit function and/or activity of PD-1, PD-L1 and/or PD-L2; (ii) an antimetabolite chemotherapeutic (e.g., 5-fluorouracil, methotrexate, capecitabine, azacitidine, 6-diazo-5-oxo-L-norleucine (DON), azaserine and acivicin), a topoisomerase inhibitor (e.g., irinotecan, topotecan, etoposide, doxorubicin, etc.); and (iii) a protein translation inhibitor (e.g., silvestrol and omacetaxine) ribosome biogenesis inhibitors (e.g., diazaborine, lamotrigine and ribozinoindoles), inhibitors of rRNA and/or tRNA synthesis (e.g., quarfloxin (CX-3543) and CX-5461), an inhibitor of synthesis of amino acids (e.g., GLUD1 inhibitor R162, BCAT1 inhibitor gabapentin, glutaminase inhibitor bis-2-(5-phenylacetamido-1,2,4-thiadiazol-2-yl)ethyl sulfide (BPTES), PAGDH inhibitor NCT-503), an inhibitor of uptake of amino acids (e.g., SLC7A11 inhibitors sulfasalazine, erastin or sorafenib), a modulator of post-translational modification (e.g., glycosylation inhibitor tunicamycin, ppGalNAc-T3), a modulator of protein degradation, and a modulator of protein transport (e.g., cyclosporin A, fendiline, parbendazole, paroxetine, parthenolide, quinacrine, sertraline, spiperone, thimerosal, astemizole, perhexiline, HUN-7293, CAM741, CK147, and cotransin, etc.) or a topoisomerase inhibitor.

In one aspect, the present disclosure relates to a method for selecting a patient for a cancer treatment, the method comprising: (a) obtaining a biological sample from a subject; (b) evaluating the biological sample for the activation of a pathway selected from Mapk8ip1, Trim7, Elk1, Lrg1, Arg1, Rap1, and Ras; and (c) selecting the cancer therapy based on the evaluation of step (b), wherein cancer therapy is selected from: (i) an agent with an ability to inhibit function and/or activity of PD-1, PD-L1 and/or PD-L2; (ii) an antimetabolite chemotherapeutic (e.g., 5-fluorouracil, methotrexate, capecitabine, azacitidine, 6-diazo-5-oxo-L-norleucine (DON), azaserine and acivicin), a topoisomerase inhibitor (e.g., irinotecan, topotecan, etoposide, doxorubicin, etc.); and (iii) a protein translation inhibitor (e.g., silvestrol and omacetaxine) ribosome biogenesis inhibitors (e.g., diazaborine, lamotrigine and ribozinoindoles), inhibitors of rRNA and/or tRNA synthesis (e.g., quarfloxin (CX-3543) and CX-5461), an inhibitor of synthesis of amino acids (e.g., GLUD1 inhibitor R162, BCAT1 inhibitor gabapentin, glutaminase inhibitor bis-2-(5-phenylacetamido-1,2,4-thiadiazol-2-yl)ethyl sulfide (BPTES), PAGDH inhibitor NCT-503), an inhibitor of uptake of amino acids (e.g., SLC7A11 inhibitors sulfasalazine, erastin or sorafenib), a modulator of post-translational modification (e.g., glycosylation inhibitor tunicamycin, ppGalNAc-T3), a modulator of protein degradation, and a modulator of protein transport (e.g., cyclosporin A, fendiline, parbendazole, paroxetine, parthenolide, quinacrine, sertraline, spiperone, thimerosal, astemizole, perhexiline, HUN-7293, CAM741, CK147, and cotransin, etc.) or a topoisomerase inhibitor.

In one aspect, the present disclosure relates to a method of treating cancer, the method comprising: (a) obtaining a biological sample from a subject; (b) evaluating the biological sample for the activation of a pathway selected from Mapk8ip1, Trim7, Elk1, Lrg1, Arg1, Rap1, and Ras; and (c) selecting the cancer therapy selected from: (i) an agent with an ability to inhibit function and/or activity of PD-1, PD-L1 and/or PD-L2; (ii) an antimetabolite chemotherapeutic (e.g., 5-fluorouracil, methotrexate, capecitabine, azacitidine, 6-diazo-5-oxo-L-norleucine (DON), azaserine and acivicin), a topoisomerase inhibitor (e.g., irinotecan, topotecan, etoposide, doxorubicin, etc.); and (iii) a protein translation inhibitor (e.g., silvestrol and omacetaxine) ribosome biogenesis inhibitors (e.g., diazaborine, lamotrigine and ribozinoindoles), inhibitors of rRNA and/or tRNA synthesis (e.g., quarfloxin (CX-3543) and CX-5461), an inhibitor of synthesis of amino acids (e.g., GLUD1 inhibitor R162, BCAT1 inhibitor gabapentin, glutaminase inhibitor bis-2-(5-phenylacetamido-1,2,4-thiadiazol-2-yl)ethyl sulfide (BPTES), PAGDH inhibitor NCT-503), an inhibitor of uptake of amino acids (e.g., SLC7A11 inhibitors sulfasalazine, erastin or sorafenib), a modulator of post-translational modification (e.g., glycosylation inhibitor tunicamycin, ppGalNAc-T3), a modulator of protein degradation, and a modulator of protein transport (e.g., cyclosporin A, fendiline, parbendazole, paroxetine, parthenolide, quinacrine, sertraline, spiperone, thimerosal, astemizole, perhexiline, HUN-7293, CAM741, CK147, and cotransin, etc.) or a topoisomerase inhibitor; and (d) administering the cancer therapy with an ability to inhibit function and/or activity of PD-1, PD-L1 and/or PD-L2; and optionally administering the therapy selected in step (c)(ii) and (c)(iii).

In one aspect, the present disclosure relates to a method of treating a cancer in a subject in need thereof, the method comprising administering a cancer therapy is selected from: an antimetabolite chemotherapeutic, a topoisomerase inhibitor, a protein translation inhibitor, a ribosome biogenesis inhibitor, an inhibitor of rRNA and/or tRNA synthesis, an inhibitor of synthesis of amino acids, an inhibitor of uptake of amino acids, a modulator of post-translational modification, a modulator of protein degradation, a modulator of protein transport, a topoisomerase inhibitor, wherein the subject has received or is receiving an anticancer treatment with an ability to inhibit function and/or activity of PD-1, PD-L1 and/or PD-L2, and wherein the subject has developed a lack of response, resistance or recalcitrance to the cancer therapy with an ability to inhibit function and/or activity of PD-1, PD-L1 and/or PD-L2; and wherein pathway selected from Mapk8ip1, Trim7, Elk1, Lrg1, Arg1, Rap1, and Ras is upregulated in the at least one tumor cell of the subject compared to a healthy tissue, a prior biological sample obtained from the subject, or another biological sample from patient that is known to be sensitive to anti-PD-1 therapy.

In one aspect, the present disclosure relates to a method of treating a cancer in a subject in need thereof, the method comprising: (a) administering an anticancer treatment with an ability to inhibit function and/or activity of PD-1, PD-L1 and/or PD-L2; (b) evaluating anti-tumor response with the anticancer treatment with an ability to inhibit function and/or activity of PD-1, PD-L1 and/or PD-L2 by monitoring a tumor size reduction in the subject; (c) administering Trim7 modulator and/or a proteasome inhibitor if a lack of tumor size reduction is observed; (d) re-evaluating anti-tumor response with the anticancer treatment with an ability to inhibit function and/or activity of PD-1, PD-L1 and/or PD-L2 by monitoring the tumor reduction in the subject; and (e) withdrawing Trim7 modulator administration if a tumor size reduction is observed. In embodiments, the proteasome inhibitor is selected from bortezomib, carfilzomib, ixazomib, oprozomib, delanzomib and marizomib.

In one aspect, the present disclosure relates to a method of treating a cancer in a subject in need thereof, the method comprising: (a) administering an anticancer treatment with an ability to inhibit function and/or activity of PD-1, PD-L1 and/or PD-L2; (b) evaluating anti-tumor response with the anticancer treatment with an ability to inhibit function and/or activity of PD-1, PD-L1 and/or PD-L2 using the steps of: (i) obtaining a biological sample from the subject; (ii) evaluating the biological sample for the overexpression and/or activation of TRIM7; (c) administering Trim7 modulator and/or a proteasome inhibitor if an overexpression and/or activation of TRIM7 is observed; (d) re-evaluating anti-tumor response with the anticancer treatment with an ability to inhibit function and/or activity of PD-1, PD-L1 and/or PD-L2 using the steps of: (i) obtaining a biological sample from the subject; (ii) evaluating the biological sample for the overexpression and/or activation of TRIM7; and (e) withdrawing Trim7 modulator administration if an overexpression and/or activation of TRIM7 is not observed. In embodiments, the proteasome inhibitor is selected from bortezomib, carfilzomib, ixazomib, oprozomib, delanzomib and marizomib.

In one aspect, the present disclosure relates to a method of treating a cancer in a subject in need thereof, the method comprising: (a) administering an anticancer treatment with an ability to inhibit function and/or activity of PD-1, PD-L1 and/or PD-L2; (b) evaluating overexpression and/or activation of TRIM7 using the steps of: (i) obtaining a biological sample from the subject; and (ii) evaluating the biological sample for the overexpression and/or activation of TRIM7; (c) administering Trim7 modulator and/or a proteasome inhibitor if an overexpression and/or activation of TRIM7 is observed; (d) re-evaluating overexpression and/or activation of TRIM7 using the steps of: (i) obtaining a biological sample from the subject; (ii) evaluating the biological sample for the overexpression and/or activation of TRIM7; and (e) withdrawing Trim7 modulator administration if an overexpression and/or activation of TRIM7 is not observed. In embodiments, the proteasome inhibitor is selected from bortezomib, carfilzomib, ixazomib, oprozomib, delanzomib and marizomib.

In embodiments, the Trim7 modulator is a Trim 7 inhibitor. In embodiments, the Trim7 modulator is selected a small interference RNA (siRNA), a short hairpin RNA (shRNA), a microRNA (miRNA), an antisense RNA, a guide RNA (gRNA), a small molecule, an antibody, a peptide, and a peptidomimetic. In embodiments, the small interference RNA (siRNA), the short hairpin RNA (shRNA), the microRNA (miRNA), the antisense RNA, or the guide RNA (gRNA) inhibit the production of Trim7 protein. In embodiments, the peptidomimetic mimics a target of Trim7 and thereby inhibits the activity of Trim7.

In embodiments, the Trim7 modulator is an mitogen- and stress-activated kinase 1 (MSK1) inhibitor, wherein the MSK1 inhibitor modulates Trim7 via downstream effect of an inhibition of MSK1. In embodiments, the MSK1 inhibitor is selected from Ro 31-8220, SB-747651A, and H89. In embodiments, the MSK1 inhibitor is SB-747651A.

Any aspects disclosed herein may be combined with any other aspects.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1A and FIG. 1B illustrate the generation of anti-PD-1-resistant CT26 tumors. FIG. 1A shows a schematic representation of the method used to generation of anti-PD-1-resistant CT26 tumors. Briefly, BALB/C mice were inoculated with 500,000 murine colon carcinoma CT26 cells, and when average tumor volume reached mm 3 (indicating day 0), mice were treated with an anti-PD-1 (clone RMP1-14; BioXcell) antibody. Tumors were excised and surviving tumor cells were dissociated and cultures in vitro. These cells were called “1st round,” “1st generation,” or “F1 generation” cells. 1st generation cells were inoculated in BALB/C mice, and following another treatment course of anti-PD-1 “2nd round,” “2nd generation,” or “F2 generation” cells were isolated. Following two more rounds, (a total of 4 rounds) of anti-PD-1 selection, “4th round,” “4th generation,” or “F4 generation” cells were isolated. FIG. 1B shows a graph comparing the efficacy of an anti-PD-1 antibody (100 μg anti-PD-1 clone RMP1-14; BioXcell) in BALB/C mice harboring CT26 parental cells and PD-1 resistant cells. BALB/C mice were inoculated with CT26 parental cells and PD-1 resistant 4th generation cells in rear flanks. When the starting tumor volume (STV) reached 80-100 mm³, mice were randomly divided in the following two treatment groups: (1) vehicle (PBS), and (2) anti-PD-1 antibody. Mice were given a series of intraperitoneal injections of vehicle or 100 μg anti-PD-1 (clone RMP1-14; BioXcell) on days 0, 3, and 6. Tumor volumes were measured on indicated days.

FIG. 2A to FIG. 2D show the transcriptomic profiling of anti-PD-1 resistant cell lines using RNA-seq. FIG. 2A (Top Panel) shows the Principal Component Analysis (PCA) that spatially separated the samples based on transcriptome expression. FIG. 2A (Bottom Panel) shows the Differentially Expressed Genes (DEGs) that were determined between the groups (parent vs. 2nd generation, parent vs. 4th generation, 2nd generation vs. 4th generation), and plotted in the heatmap. Hierarchical clustering was performed to rank order genes on each row, which separated genes into 2 major clusters in each comparison, where a subset of gene expression was lower (blue) in one dataset and higher (red) in the other. FIG. 2B shows the up- or downregulated genes in each dataset. The genes were input into PANTHER to identify Gene Ontologies (GO) associated with each gene set. Gene sets are shown with associated p-values. FIG. 2C (Top Panel) shows a Venn diagram of overlap in gene expression between all datasets. FIG. 2C (Bottom Panel) shows the transcripts per million (TPM; normalized expression data) at select genes, demonstrating higher baseline expression of genes associated with PD-L1, antigen processing/presentation, protein translation, ER trafficking; in some datasets over others. FIG. 2D shows the transcripts per million (TPM; normalized expression data) of select genes, demonstrating higher baseline expression of genes associated with PD-L1, antigen processing/presentation, protein translation, ER trafficking; in some datasets over others.

FIG. 3 illustrates that there is a discordance in gene expression and cell surface protein expression in PD-L1/2 MHC Class I and B2M. Parental and 4th generation anti-PD-1 resistant cells were harvested from culture and analyzed by flow cytometry for surface expression of PD-L1, PD-L2, MHC Class I, and β2 microglobulin (B2M). Gates were drawn as shown, and shown above each plot is the percentage of cells in each gate, and to the right of each percentage, the MFI (mean fluorescent intensity) of each marker.

FIG. 4A and FIG. 4B show the transcriptomic profiling of anti-PD-1 resistant cell lines. FIG. 4A shows the in comparison of 2nd- and 4th generation anti-PD-1 resistance cell lines with B16.F10 is a murine melanoma tumor, which served as a model of anti-PD-1 “primary resistance,” as these tumors are not responsive to anti-PD-1 therapy. Cells were cultured for 24 hours in IFNγ to assess in vitro responsiveness. This mimics how tumor cells respond in vivo, as immune cell infiltrate and secrete effector cytokines like IFNγ. FIG. 4A (Left Panel) shows the DEGs that were identified between untreated and IFNγ treated parental CT26. Of these, 338 genes had usable data from the other datasets; and those values are shown in the other columns. Log 2 fold-change was plotted in the heatmap and genes are hierarchical clustered based on parental CT26. Genes separated into 3 major clusters. FIG. 4A (Right Panel) shows the GO pathways associated with the DEGs. Associated genes were input into PANTHER to identify pathways associated with the dysregulated genes. FIG. 4B shows the transcripts per million (TPM; normalized expression data) at select genes, demonstrating that although CT26 anti-PD-1 resistant cells have baseline hyperactivation of type I and type II interferons, when those cells are challenged with IFNγ, those cells downregulate these genes.

FIG. 5A to FIG. 5D illustrate the paradoxical dysregulation of certain genes associated with the acquired resistance to anti-PD-1. The genes encoding CD274 (FIG. 5A) and B2m (FIG. 5B), which are overexpressed in the 4th generation anti-PD-1 resistant cells, are overexpressed in wild type CT26 cells but are repressed in the 4th generation anti-PD-1 resistant cells in the presence of IFNγ. On the other hand, the genes encoding Trim7 (FIG. 5C) and Lrg1 (FIG. 5D), which are repressed in the 4th generation anti-PD-1 resistant cells, are repressed in wild type CT26 cells but are but are overexpressed in the 4th generation anti-PD-1 resistant cells in response to IFNγ. Second generation anti-PD-1 resistant cells show an intermediate phenotype.

FIG. 6A and FIG. 6F show the identification of driver genes involved in acquired resistance to anti-PD-1 and the functional pathways affected by the driver genes. FIG. 6A, Panels 1-4 show the methodology used for the identification of the driver genes. FIG. 6A (Panel 1) shows that 1,999 genes are downregulated and 3607 genes are upregulated the 4th generation anti-PD-1 resistant cells in the presence of IFNγ compared to the CT26 cells. FIG. 6A (Panel 2) shows that 1,060 genes that are downregulated the 4th generation anti-PD-1 resistant cells but are upregulated the CT26 cells. FIG. 6A (Panel 3) shows that 688 genes that are downregulated the 4th generation anti-PD-1 resistant cells compared to the CT26 cells as revealed by sorting according to responsiveness to IFNγ. FIG. 6A (Panel 4) shows that 70 genes that upregulated in vivo in the 4th generation anti-PD-1 resistant cells compared to the CT26 cells. FIG. 6B shows the gene ontology (GO) pathways associated with the genes identified using the methodology of FIG. 6A. FIG. 6C shows the functional pathways affected by the TRIM family of proteins. FIG. 6D shows the functional pathways in which Elk1 and c-Jun play a role. FIG. 6E shows the functional connections between Lrg1, B2m and Arg1 with other genes. FIG. 6F shows the levels of expression of Elk1 in tumors and surrounding normal tissue in the Cancer Genome Atlas (TCGA) cancer genomics program.

FIG. 7A to FIG. 7D show that Stat1 (FIG. 7A), Stat2 (FIG. 7B), Irf1 (FIG. 7C) and Tap1 (FIG. 7D) genes, which are overexpressed in response to IFNγ, are overexpressed in the 4th generation anti-PD-1 resistant cells, and are repressed in the 4th generation anti-PD-1 resistant cells in the presence of IFNγ. Second generation anti-PD-1 resistant cells show an intermediate phenotype.

FIG. 8A and FIG. 8B show the pathway analysis of the differentially expressed genes, which led to the identification of the Ras and Rap1 signaling pathways. FIG. 8A shows the identification of pathways using the WEB-based GEne SeT AnaLysis Toolkit (WebGestalt) using the top 1,000 genes from FIG. 6A (Panel 2). FIG. 8B shows a Volcano plot of the data presented in FIG. 8A. FIG. 8C shows the RAS signaling pathway. The convergence with Raf/Mek/Erk signaling is shown using ovals. FIG. 8D shows the RAP1 signaling pathway. The convergence with Raf/Mek/Erk signaling is shown using ovals.

FIG. 9A to FIG. 9C show that Ccl5 (RANTES) (FIG. 9A), Cxcl10 (IP-10) (FIG. 9B), and Ifnb1 (FIG. 9C) genes, which are overexpressed in response to IFNγ, are overexpressed in the 4th generation anti-PD-1 resistant cells, and are repressed in the 4th generation anti-PD-1 resistant cells in the presence of IFNγ. Second generation anti-PD-1 resistant cells show an intermediate phenotype.

### DETAILED DESCRIPTION

The current disclosure is based, in part, on surprising discovery that upregulation of one or more genes associated with the following Gene Ontology (GO) functions are associated with resistance to anti-PD-1 therapy (acquired or primary resistance): positive regulation of IκB kinase/NFκB signaling, type I IFN signaling pathways, positive regulation of IFNα production, positive regulation of defense response, positive regulation of IFNβ production, and regulation of inflammatory response. This is surprising, inter alia, because these pathways are all are known to be involved in sensitivity of cancer cells to anti-PD-1 antibodies. Karachaliou et al., Interferon gamma, an important marker of response to immune checkpoint blockade in non-small cell lung cancer and melanoma patients, Ther Adv Med Oncol. 10: 1758834017749748 (2018); Rafique et al., Immune Checkpoint Blockade and Interferon-α in Melanoma, Semin Oncol. 42(3): 436-447 (2015); Uehara et al., Intratumoral injection of IFN-β induces chemokine production in melanoma and augments the therapeutic efficacy of anti-PD-L1 mAb, Biochemical and Biophysical Research Communications 490(2) 521-527 (2017). Thus, these results establish biomarkers associated with acquired and primary resistance to anti-PD-1 therapy.

In embodiments, these results establish biomarkers associated with acquired and primary resistance to anti-PD-1 therapy. Therefore, based on these biomarkers, in embodiments disclosed herein a patient may be selected for treatment with an anti-PD-1 therapy based on evaluating the sample for the presence, absence, or level of genes associated with one or more gene ontology (GO) pathways disclosed herein from a biological sample from the patient. For example, in embodiments, the observed upregulation or downregulation of one or more genes associated with in various GO functions disclosed herein may be used to diagnose resistance to anti-PD-1 therapy (acquired or primary resistance).

Single-cell RNA sequencing of tumors, which have developed acquired resistance to PD-1 inhibitory antibodies, disclosed herein demonstrated the progressive acquisition of a transcriptionally hyperactive phenotype. Specifically, a larger number of transcripts were upregulated than downregulated in PD-1 acquired resistant tumors than the parental PD-1 antibody sensitive tumor. This observation suggests, without wishing to be bound by theory, that acquired resistance is an active process, in which tumor cells, which upregulate genes in certain pathways, acquire a survival advantage as compared to those cells that do not upregulate, or which downregulate, overall transcriptional activity. Accordingly, in embodiments, the tumors that have acquired resistance to checkpoint inhibitors, including PD-1 or PD-L1 blocking agents, may have increased sensitivity to certain classes of chemotherapy which target transcriptionally active cells. Therefore, in aspects, the tumors that have acquired resistance to checkpoint inhibitors, may be treated with the classes of chemotherapy which target transcriptionally active cells. In embodiments, the classes of chemotherapy include antimetabolite chemotherapeutics (e.g., 5-fluorouracil, methotrexate, capecitabine, azacitidine, etc.) or topoisomerase inhibitors (e.g., irinotecan, topotecan, etoposide, doxorubicin, etc.). In embodiments, the classes of chemotherapy may be used as neoadjuvant therapy or as adjuvant therapy.

In addition, the data presented herein demonstrate that while acquired resistant tumors are characterized by a transcriptionally hyperactive phenotype, many of the transcripts that are upregulated are not accompanied by an increase in corresponding protein expression. For example, the genes associated with CD274 (PD-L1), beta 2 macroglobulin, and other transcripts associated with interferon sensitivity and antigen presentation are upregulated in acquired resistant tumors, however there is not a corresponding increase in the amount of PD-L1 or beta 2 macroglobulin protein expression in acquired resistant tumors. Together, these findings suggest, inter alia, that acquired resistant tumors are attempting to upregulate many of the key genes that would drive increased sensitivity to an anti-tumor immune response, but that an acquired defect in a post-transcriptional protein or proteins has disrupted the response. For example, the increased PD-L1 mRNA levels would be expected to translate to increased levels of the PD-L1 protein. Accordingly, in embodiments, the modulators of one or more processes including: protein translation (e.g., assembly and/or function of ribosomal complex, adequate expression and/or function of tRNA, adequate synthesis and/or uptake of amino acids, etc.), post-translational modification (e.g., decoration of the translated protein with carbohydrates important for function or protection from degradation), or transport mechanisms (e.g., post-translational peptide processing, signal peptide recognition and cleavage, transport through the ER/Golgi network, etc.) may be helpful as combining defects in post-translational processes to create synthetic lethal phenotypes in cancer cell populations developing resistance to PD-1 or PD-L1 blocking agents. In embodiments, the classes of chemotherapy may be used as neoadjuvant therapy or as adjuvant therapy.

### Methods of Determining a Cancer Treatment for a Patient; Methods of Selecting a Patient for a Cancer Treatment; and Methods of Treatment

Accordingly, in one aspect, the present disclosure relates to a method of determining a cancer treatment for a patient, the method comprising: (a) obtaining a biological sample from a subject; (b) evaluating the sample for the presence, absence, or level of one or more gene associated with a gene ontology (GO) pathway selected from: (i) positive regulation of cell cycle process, regulation of G1/S transition, regulation of cell division, regulation of cell proliferation, positive regulation of IκB kinase/NFκB signaling, type I IFN signaling pathway, cellular response to IFNγ, positive regulation of IFNα production, positive regulation of defense response, positive regulation of IFNβ production, regulation of inflammatory response, regulation of innate immune response, negative regulation of antigen processing and presentation, and antigen processing, presentation of endogenous peptides via MHC class I; and/or (ii) phospholipid efflux, negative regulation of fibrinolysis, chylomicron assembly, plasma membrane repair, SRP-dependent co-translational protein targeting to membrane, ribosomal small subunit assembly, phospholipid efflux, regulation of translation, mitochondrial respiratory chain complex I, mitochondrial translational elongation, DNA-dependent DNA replication, and ATP biosynthetic process; and (c) selecting the cancer therapy with an ability to inhibit function and/or activity of PD-1, PD-L1 and/or PD-L2 based on the evaluation of step (b).

In one aspect, the present disclosure relates to a method of treating cancer, the method comprising: (a) obtaining a biological sample from a subject; (b) evaluating the sample for the presence, absence, or level of one or more genes associated with a gene ontology (GO) pathway selected from: (i) positive regulation of cell cycle process, regulation of G1/S transition, regulation of cell division, regulation of cell proliferation, positive regulation of IκB kinase/NFκB signaling, type I IFN signaling pathway, cellular response to IFNγ, positive regulation of IFNα production, positive regulation of defense response, positive regulation of IFNβ production, regulation of inflammatory response, regulation of innate immune response, negative regulation of antigen processing and presentation, and antigen processing, presentation of endogenous peptides via MHC class I; and/or (ii) phospholipid efflux, negative regulation of fibrinolysis, chylomicron assembly, plasma membrane repair, SRP-dependent co-translational protein targeting to membrane, ribosomal small subunit assembly, phospholipid efflux, regulation of translation, mitochondrial respiratory chain complex I, mitochondrial translational elongation, DNA-dependent DNA replication, and ATP biosynthetic process; and (c) selecting the cancer therapy selected from: (i) an agent with an ability to inhibit function and/or activity of PD-1, PD-L1 and/or PD-L2; (ii) an antimetabolite chemotherapeutic (e.g., 5-fluorouracil, methotrexate, capecitabine, azacitidine, 6-diazo-5-oxo-L-norleucine (DON), azaserine and acivicin), a topoisomerase inhibitor (e.g., irinotecan, topotecan, etoposide, doxorubicin, etc.); and (iii) a protein translation inhibitor (e.g., silvestrol and omacetaxine) ribosome biogenesis inhibitors (e.g., diazaborine, lamotrigine and ribozinoindoles), inhibitors of rRNA and/or tRNA synthesis (e.g., quarfloxin (CX-3543) and CX-5461), an inhibitor of synthesis of amino acids (e.g., GLUD1 inhibitor R162, BCAT1 inhibitor gabapentin, glutaminase inhibitor bis-2-(5-phenylacetamido-1,2,4-thiadiazol-2-yl)ethyl sulfide (BPTES), PAGDH inhibitor NCT-503), an inhibitor of uptake of amino acids (e.g., SLC7A11 inhibitors sulfasalazine, erastin or sorafenib), a modulator of post-translational modification (e.g., glycosylation inhibitor tunicamycin, ppGalNAc-T3), a modulator of protein degradation, and a modulator of protein transport (e.g., cyclosporin A, fendiline, parbendazole, paroxetine, parthenolide, quinacrine, sertraline, spiperone, thimerosal, astemizole, perhexiline, HUN-7293, CAM741, CK147, and cotransin); and (d) administering the cancer therapy with an ability to inhibit function and/or activity of PD-1, PD-L1 and/or PD-L2; and optionally administering the therapy selected in step (c)(ii) and (c)(iii).

In non-limiting embodiments, administering the cancer therapy with an ability to inhibit function and/or activity of PD-1, PD-L1 and/or PD-L2 is continued if the upregulation of genes associated with a GO pathway selected from positive regulation of cell cycle process, regulation of G1/S transition, regulation of cell division, regulation of cell proliferation, positive regulation of IκB kinase/NFκB signaling, type I IFN signaling pathway, cellular response to IFNγ, positive regulation of IFNα production, positive regulation of defense response, positive regulation of IFNβ production, regulation of inflammatory response, regulation of innate immune response, negative regulation of antigen processing and presentation, and antigen processing, presentation of endogenous peptides via MHC class I is not observed and/or if the downregulation of genes associated with a GO pathway selected from phospholipid efflux, negative regulation of fibrinolysis, chylomicron assembly, plasma membrane repair, SRP-dependent co-translational protein targeting to membrane, ribosomal small subunit assembly, phospholipid efflux, regulation of translation, mitochondrial respiratory chain complex I, mitochondrial translational elongation, DNA-dependent DNA replication, and ATP biosynthetic process is not observed.

In non-limiting embodiments, administering the cancer therapy with an ability to inhibit function and/or activity of PD-1, PD-L1 and/or PD-L2 is continued if the upregulation of genes associated with a GO pathway selected from positive regulation of cell cycle process, regulation of G1/S transition, regulation of cell division, regulation of cell proliferation, positive regulation of IκB kinase/NFκB signaling, type I IFN signaling pathway, cellular response to IFNγ, positive regulation of IFNα production, positive regulation of defense response, positive regulation of IFNβ production, regulation of inflammatory response, regulation of innate immune response, negative regulation of antigen processing and presentation, and antigen processing, presentation of endogenous peptides via MHC class I is observed and/or if the downregulation of genes associated with a GO pathway selected from phospholipid efflux, negative regulation of fibrinolysis, chylomicron assembly, plasma membrane repair, SRP-dependent co-translational protein targeting to membrane, ribosomal small subunit assembly, phospholipid efflux, regulation of translation, mitochondrial respiratory chain complex I, mitochondrial translational elongation, DNA-dependent DNA replication, and ATP biosynthetic process is observed, wherein the supplementation of administration of cancer therapy selected from: (i) an agent with an ability to inhibit function and/or activity of PD-1, PD-L1 and/or PD-L2; (ii) an antimetabolite chemotherapeutic (e.g., 5-fluorouracil, methotrexate, capecitabine, azacitidine, 6-diazo-5-oxo-L-norleucine (DON), azaserine and acivicin), a topoisomerase inhibitor (e.g., irinotecan, topotecan, etoposide, doxorubicin, etc.); and (iii) a protein translation inhibitor (e.g., silvestrol and omacetaxine) ribosome biogenesis inhibitors (e.g., diazaborine, lamotrigine and ribozinoindoles), inhibitors of rRNA and/or tRNA synthesis (e.g., quarfloxin (CX-3543) and CX-5461), an inhibitor of synthesis of amino acids (e.g., GLUD1 inhibitor R162, BCAT1 inhibitor gabapentin, glutaminase inhibitor bis-2-(5-phenylacetamido-1,2,4-thiadiazol-2-yl)ethyl sulfide (BPTES), PAGDH inhibitor NCT-503), an inhibitor of uptake of amino acids (e.g., SLC7A11 inhibitors sulfasalazine, erastin or sorafenib), a modulator of post-translational modification (e.g., glycosylation inhibitor tunicamycin, ppGalNAc-T3), a modulator of protein degradation, and a modulator of protein transport (e.g., cyclosporin A, fendiline, parbendazole, paroxetine, parthenolide, quinacrine, sertraline, spiperone, thimerosal, astemizole, perhexiline, HUN-7293, CAM741, CK147, and cotransin) is carried out.

In non-limiting embodiments, administering the cancer therapy with an ability to inhibit function and/or activity of PD-1, PD-1.1 and/or PD-L2 is not continued if the upregulation of genes associated with a GO pathway selected from positive regulation of cell cycle process, regulation of G1/S transition, regulation of cell division, regulation of cell proliferation, positive regulation of IκB kinase/NFκB signaling, type I IFN signaling pathway, cellular response to IFNγ, positive regulation of IFNα production, positive regulation of defense response, positive regulation of IFNβ production, regulation of inflammatory response, regulation of innate immune response, negative regulation of antigen processing and presentation, and antigen processing, presentation of endogenous peptides via MHC class I is observed and/or if the downregulation of genes associated with a GO pathway selected from phospholipid efflux, negative regulation of fibrinolysis, chylomicron assembly, plasma membrane repair, SRP-dependent co-translational protein targeting to membrane, ribosomal small subunit assembly, phospholipid efflux, regulation of translation, mitochondrial respiratory chain complex I, mitochondrial translational elongation, DNA-dependent DNA replication, and ATP biosynthetic process is observed, wherein the administration of cancer therapy selected from: (i) an agent with an ability to inhibit function and/or activity of PD-1, PD-L1 and/or PD-L2; (ii) an antimetabolite chemotherapeutic (e.g., 5-fluorouracil, methotrexate, capecitabine, azacitidine, 6-diazo-5-oxo-L-norleucine (DON), azaserine and acivicin), a topoisomerase inhibitor (e.g., irinotecan, topotecan, etoposide, doxorubicin, etc.); and (iii) a protein translation inhibitor (e.g., silvestrol and omacetaxine) ribosome biogenesis inhibitors (e.g., diazaborine, lamotrigine and ribozinoindoles), inhibitors of rRNA and/or tRNA synthesis (e.g., quarfloxin (CX-3543) and CX-5461), an inhibitor of synthesis of amino acids (e.g., GLUD1 inhibitor R162, BCAT1 inhibitor gabapentin, glutaminase inhibitor bis-2-(5-phenylacetamido-1,2,4-thiadiazol-2-yl)ethyl sulfide (BPTES), PAGDH inhibitor NCT-503), an inhibitor of uptake of amino acids (e.g., SLC7A11 inhibitors sulfasalazine, erastin or sorafenib), a modulator of post-translational modification (e.g., glycosylation inhibitor tunicamycin, ppGalNAc-T3), a modulator of protein degradation, and a modulator of protein transport (e.g., cyclosporin A, fendiline, parbendazole, paroxetine, parthenolide, quinacrine, sertraline, spiperone, thimerosal, astemizole, perhexiline, HUN-7293, CAM741, CK147, and cotransin) is carried out.

In embodiments, an upregulation of one or more genes associated with a GO pathway listed in (i) compared to a healthy tissue indicates a lack of response, resistance or recalcitrance to the cancer therapy with an ability to inhibit function and/or activity of PD-1, PD-L1 and/or PD-L2. In embodiments, an upregulation of one or more genes associated with a GO pathway listed in (i) compared to another biological sample from patient that is known to be sensitive to anti-PD-1 therapy indicates a lack of response, resistance or recalcitrance to the cancer therapy with an ability to inhibit function and/or activity of PD-1, PD-L1 and/or PD-L2. In embodiments, an upregulation of one or more genes associated with a GO pathway listed in (i) compared to a prior biological sample obtained from the subject indicates a development of resistance, a lack of response, or recalcitrance to the cancer therapy with an ability to inhibit function and/or activity of PD-1, PD-L1 and/or PD-L2.

In embodiments, a lack of upregulation of one or more genes associated with a GO pathway listed in (i) compared to a healthy tissue indicates a response to the cancer therapy with an ability to inhibit function and/or activity of PD-1, PD-L1 and/or PD-L2. In embodiments, a lack of upregulation of one or more genes associated with a GO pathway listed in (i) compared to another biological sample from patient that is known to be sensitive to anti-PD-1 therapy indicates a response to the cancer therapy with an ability to inhibit function and/or activity of PD-1, PD-L1 and/or PD-L2. In embodiments, a lack of upregulation of one or more genes associated with a GO pathway listed in (i) compared to a prior biological sample obtained from the subject indicates a response to the cancer therapy with an ability to inhibit function and/or activity of PD-1, PD-L1 and/or PD-L2.

In embodiments, a downregulation of one or more genes associated with a GO pathway listed in (ii) compared to a healthy tissue indicates a lack of response, resistance or recalcitrance to the cancer therapy with an ability to inhibit function and/or activity of PD-1, PD-L1 and/or PD-L2. In embodiments, a downregulation of one or more genes associated with a GO pathway listed in (ii) compared to another biological sample from patient that is known to be sensitive to anti-PD-1 therapy indicates a lack of response, resistance or recalcitrance to the cancer therapy with an ability to inhibit function and/or activity of PD-1, PD-L1 and/or PD-L2. In embodiments, a downregulation of one or more genes associated with a GO pathway listed in (ii) compared to a prior biological sample obtained from the subject indicates a development of resistance, a lack of response, or recalcitrance to the cancer therapy with an ability to inhibit function and/or activity of PD-1, PD-L1 and/or PD-L2.

In embodiments, a lack of downregulation of one or more genes associated with a GO pathway listed in (ii) compared to a healthy tissue indicates a response to the cancer therapy with an ability to inhibit function and/or activity of PD-1, PD-L1 and/or PD-L2. In embodiments, a lack of downregulation of one or more genes associated with a GO pathway listed in (ii) compared to another biological sample from patient that is known to be sensitive to anti-PD-1 therapy indicates a response to the cancer therapy with an ability to inhibit function and/or activity of PD-1, PD-L1 and/or PD-L2. In embodiments, a lack of downregulation of one or more genes associated with a GO pathway listed in (ii) compared to a prior biological sample obtained from the subject indicates a response to the cancer therapy with an ability to inhibit function and/or activity of PD-1, PD-L1 and/or PD-L2.

In embodiments, a lack of upregulation of one or more genes associated with a GO pathway compared to a prior biological sample from the subject indicates a response to the cancer therapy with an ability to inhibit function and/or activity of PD-1, PD-L1 and/or PD-L2, wherein the GO pathway is selected from (a) cellular response to IFNγ, (b) negative regulation of antigen processing and presentation, (c) type I IFN signaling pathway, (d) positive regulation of IκB kinase/NFκB signaling, and antigen processing, and (e) presentation of endogenous peptides via MHC class I. In embodiments, a lack of upregulation of one or more genes associated with a GO pathway compared to a patient that is known to be sensitive to anti-PD-1 therapy indicates a response to the cancer therapy with an ability to inhibit function and/or activity of PD-1, PD-L1 and/or PD-L2, wherein the GO pathway is selected from (a) cellular response to IFNγ, (b) negative regulation of antigen processing and presentation, (c) type I IFN signaling pathway, (d) positive regulation of IκB kinase/NFκB signaling, and antigen processing, and (e) presentation of endogenous peptides via MHC class I. In embodiments, a lack of upregulation of one or more genes associated with a GO pathway compared to a standard indicates a response to the cancer therapy with an ability to inhibit function and/or activity of PD-1, PD-L1 and/or PD-L2, wherein the GO pathway is selected from (a) cellular response to IFNγ, (b) negative regulation of antigen processing and presentation, (c) type I IFN signaling pathway, (d) positive regulation of IκB kinase/NFκB signaling, and antigen processing, and (e) presentation of endogenous peptides via MHC class I.

In embodiments, an upregulation of one or more genes associated with a GO pathway compared to a prior biological sample from the subject indicates resistance, a lack of response, or recalcitrance to the cancer therapy with an ability to inhibit function and/or activity of PD-1, PD-L1 and/or PD-L2, wherein the GO pathway is selected from (a) cellular response to IFNγ, (b) negative regulation of antigen processing and presentation, (c) type I IFN signaling pathway, (d) positive regulation of IκB kinase/NFκB signaling, and antigen processing, and (e) presentation of endogenous peptides via MHC class I. In embodiments, an upregulation of one or more genes associated with a GO pathway compared to a patient that is known to be sensitive to anti-PD-1 therapy indicates resistance, a lack of response, or recalcitrance to the cancer therapy with an ability to inhibit function and/or activity of PD-1, PD-L1 and/or PD-L2, wherein the GO pathway is selected from (a) cellular response to IFNγ, (b) negative regulation of antigen processing and presentation, (c) type I IFN signaling pathway, (d) positive regulation of IκB kinase/NFκB signaling, and antigen processing, and (e) presentation of endogenous peptides via MHC class I. In embodiments, an upregulation of one or more genes associated with a GO pathway compared to a standard indicates resistance, a lack of response, or recalcitrance to the cancer therapy with an ability to inhibit function and/or activity of PD-1, PD-L1 and/or PD-L2, wherein the GO pathway is selected from (a) cellular response to IFNγ, (b) negative regulation of antigen processing and presentation, (c) type I IFN signaling pathway, (d) positive regulation of IκB kinase/NFκB signaling, and antigen processing, and (e) presentation of endogenous peptides via MHC class I.

In embodiments, a lack of upregulation of one or more genes associated with cellular response to IFNγ compared to a prior biological sample from the subject indicates a response to the cancer therapy with an ability to inhibit function and/or activity of PD-1, PD-L1 and/or PD-L2. In embodiments, a lack of upregulation of one or more genes associated with cellular response to IFNγ compared to a patient that is known to be sensitive to anti-PD-1 therapy indicates a response to the cancer therapy with an ability to inhibit function and/or activity of PD-1, PD-L1 and/or PD-L2. In embodiments, a lack of upregulation of one or more genes associated with cellular response to IFNγ compared to a standard indicates a response to the cancer therapy with an ability to inhibit function and/or activity of PD-1, PD-L1 and/or PD-L2.

In embodiments, an upregulation of one or more genes associated with cellular response to IFNγ compared to a prior biological sample from the subject indicates resistance, a lack of response, or recalcitrance to the cancer therapy with an ability to inhibit function and/or activity of PD-1, PD-L1 and/or PD-L2. In embodiments, an upregulation of one or more genes associated with cellular response to IFNγ compared to a patient that is known to be sensitive to anti-PD-1 therapy indicates resistance, a lack of response, or recalcitrance to the cancer therapy with an ability to inhibit function and/or activity of PD-1, PD-L1 and/or PD-L2. In embodiments, an upregulation of one or more genes associated with cellular response to IFNγ compared to a standard indicates resistance, a lack of response, or recalcitrance to the cancer therapy with an ability to inhibit function and/or activity of PD-1, PD-L1 and/or PD-L2.

In embodiments, an upregulation of one or more genes associated with type I IFN signaling pathway compared to a prior biological sample from the subject indicates resistance, a lack of response, or recalcitrance to the cancer therapy with an ability to inhibit function and/or activity of PD-1, PD-L1 and/or PD-L2. In embodiments, an upregulation of one or more genes associated with type I IFN signaling pathway compared to a patient that is known to be sensitive to anti-PD-1 therapy indicates resistance, a lack of response, or recalcitrance to the cancer therapy with an ability to inhibit function and/or activity of PD-1, PD-L1 and/or PD-L2. In embodiments, an upregulation of one or more genes associated with type I IFN signaling pathway compared to a standard indicates resistance, a lack of response, or recalcitrance to the cancer therapy with an ability to inhibit function and/or activity of PD-1, PD-L1 and/or PD-L2.

In embodiments, a lack of upregulation of one or more genes associated with type I IFN signaling pathway compared to a prior biological sample from the subject indicates a response to the cancer therapy with an ability to inhibit function and/or activity of PD-1, PD-L1 and/or PD-L2. In embodiments, a lack of upregulation of one or more genes associated with type I IFN signaling pathway compared to a patient that is known to be sensitive to anti-PD-1 therapy indicates a response to the cancer therapy with an ability to inhibit function and/or activity of PD-1, PD-L1 and/or PD-L2. In embodiments, a lack of upregulation of one or more genes associated with type I IFN signaling pathway compared to a standard indicates a response to the cancer therapy with an ability to inhibit function and/or activity of PD-1, PD-L1 and/or PD-L2.

In embodiments, an upregulation of one or more genes associated with positive regulation of cell cycle process compared to a prior biological sample from the subject indicates resistance, a lack of response, or recalcitrance to the cancer therapy with an ability to inhibit function and/or activity of PD-1, PD-L1 and/or PD-L2. In embodiments, an upregulation of one or more genes associated with positive regulation of cell cycle process compared to a patient that is known to be sensitive to anti-PD-1 therapy indicates resistance, a lack of response, or recalcitrance to the cancer therapy with an ability to inhibit function and/or activity of PD-1, PD-L1 and/or PD-L2. In embodiments, an upregulation of one or more genes associated with positive regulation of cell cycle process compared to a standard indicates resistance, a lack of response, or recalcitrance to the cancer therapy with an ability to inhibit function and/or activity of PD-1, PD-L1 and/or PD-L2.

In embodiments, a lack of upregulation of one or more genes associated with positive regulation of cell cycle process compared to a prior biological sample from the subject indicates a response to the cancer therapy with an ability to inhibit function and/or activity of PD-1, PD-L1 and/or PD-L2. In embodiments, a lack of upregulation of one or more genes associated with positive regulation of cell cycle process compared to a patient that is known to be sensitive to anti-PD-1 therapy indicates a response to the cancer therapy with an ability to inhibit function and/or activity of PD-1, PD-L1 and/or PD-L2. In embodiments, a lack of upregulation of one or more genes associated with positive regulation of cell cycle process compared to a standard indicates a response to the cancer therapy with an ability to inhibit function and/or activity of PD-1, PD-L1 and/or PD-L2.

In embodiments, an upregulation of one or more genes associated with regulation of G1/S transition compared to a prior biological sample from the subject indicates resistance, a lack of response, or recalcitrance to the cancer therapy with an ability to inhibit function and/or activity of PD-1, PD-L1 and/or PD-L2. In embodiments, an upregulation of one or more genes associated with regulation of G1/S transition compared to a patient that is known to be sensitive to anti-PD-1 therapy indicates resistance, a lack of response, or recalcitrance to the cancer therapy with an ability to inhibit function and/or activity of PD-1, PD-L1 and/or PD-L2. In embodiments, an upregulation of one or more genes associated with regulation of G1/S transition compared to a standard indicates resistance, a lack of response, or recalcitrance to the cancer therapy with an ability to inhibit function and/or activity of PD-1, PD-L1 and/or PD-L2.

In embodiments, a lack of upregulation of one or more genes associated with regulation of G1/S transition compared to a prior biological sample from the subject indicates a response to the cancer therapy with an ability to inhibit function and/or activity of PD-1, PD-L1 and/or PD-L2. In embodiments, a lack of upregulation of one or more genes associated with regulation of G1/S transition compared to a patient that is known to be sensitive to anti-PD-1 therapy indicates a response to the cancer therapy with an ability to inhibit function and/or activity of PD-1, PD-L1 and/or PD-L2. In embodiments, a lack of upregulation of one or more genes associated with regulation of G1/S transition compared to a standard indicates a response to the cancer therapy with an ability to inhibit function and/or activity of PD-1, PD-L1 and/or PD-L2.

In embodiments, an upregulation of one or more genes associated with regulation of cell division compared to a prior biological sample from the subject indicates resistance, a lack of response, or recalcitrance to the cancer therapy with an ability to inhibit function and/or activity of PD-1, PD-L1 and/or PD-L2. In embodiments, an upregulation of one or more genes associated with regulation of cell division compared to a patient that is known to be sensitive to anti-PD-1 therapy indicates resistance, a lack of response, or recalcitrance to the cancer therapy with an ability to inhibit function and/or activity of PD-1, PD-L1 and/or PD-L2. In embodiments, an upregulation of one or more genes associated with regulation of cell division compared to a standard indicates resistance, a lack of response, or recalcitrance to the cancer therapy with an ability to inhibit function and/or activity of PD-1, PD-L1 and/or PD-L2.

In embodiments, a lack of upregulation of one or more genes associated with regulation of cell division compared to a prior biological sample from the subject indicates a response to the cancer therapy with an ability to inhibit function and/or activity of PD-1, PD-L1 and/or PD-L2. In embodiments, a lack of upregulation of one or more genes associated with regulation of cell division compared to a patient that is known to be sensitive to anti-PD-1 therapy indicates a response to the cancer therapy with an ability to inhibit function and/or activity of PD-1, PD-L1 and/or PD-L2. In embodiments, a lack of upregulation of one or more genes associated with regulation of cell division compared to a standard indicates a response to the cancer therapy with an ability to inhibit function and/or activity of PD-1, PD-L1 and/or PD-L2.

In embodiments, an upregulation of one or more genes associated with regulation of cell proliferation compared to a prior biological sample from the subject indicates resistance, a lack of response, or recalcitrance to the cancer therapy with an ability to inhibit function and/or activity of PD-1, PD-L1 and/or PD-L2. In embodiments, an upregulation of one or more genes associated with regulation of cell proliferation compared to a patient that is known to be sensitive to anti-PD-1 therapy indicates resistance, a lack of response, or recalcitrance to the cancer therapy with an ability to inhibit function and/or activity of PD-1, PD-L1 and/or PD-L2. In embodiments, an upregulation of one or more genes associated with regulation of cell proliferation compared to a standard indicates resistance, a lack of response, or recalcitrance to the cancer therapy with an ability to inhibit function and/or activity of PD-1, PD-L1 and/or PD-L2.

In embodiments, a lack of upregulation of one or more genes associated with regulation of cell proliferation compared to a prior biological sample from the subject indicates a response to the cancer therapy with an ability to inhibit function and/or activity of PD-1, PD-L1 and/or PD-L2. In embodiments, a lack of upregulation of one or more genes associated with regulation of cell proliferation compared to a patient that is known to be sensitive to anti-PD-1 therapy indicates a response to the cancer therapy with an ability to inhibit function and/or activity of PD-1, PD-L1 and/or PD-L2. In embodiments, a lack of upregulation of one or more genes associated with regulation of cell proliferation compared to a standard indicates a response to the cancer therapy with an ability to inhibit function and/or activity of PD-1, PD-L1 and/or PD-L2.

In embodiments, an upregulation of one or more genes associated with positive regulation of IκB kinase/NFκB signaling compared to a prior biological sample from the subject indicates resistance, a lack of response, or recalcitrance to the cancer therapy with an ability to inhibit function and/or activity of PD-1, PD-L1 and/or PD-L2. In embodiments, an upregulation of one or more genes associated with positive regulation of IκB kinase/NFκB signaling compared to a patient that is known to be sensitive to anti-PD-1 therapy indicates resistance, a lack of response, or recalcitrance to the cancer therapy with an ability to inhibit function and/or activity of PD-1, PD-L1 and/or PD-L2. In embodiments, an upregulation of one or more genes associated with positive regulation of IκB kinase/NFκB signaling compared to a standard indicates resistance, a lack of response, or recalcitrance to the cancer therapy with an ability to inhibit function and/or activity of PD-1, PD-L1 and/or PD-L2.

In embodiments, a lack of upregulation of one or more genes associated with positive regulation of IκB kinase/NFκB signaling compared to a prior biological sample from the subject indicates a response to the cancer therapy with an ability to inhibit function and/or activity of PD-1, PD-L1 and/or PD-L2. In embodiments, a lack of upregulation of one or more genes associated with positive regulation of IκB kinase/NFκB signaling compared to a patient that is known to be sensitive to anti-PD-1 therapy indicates a response to the cancer therapy with an ability to inhibit function and/or activity of PD-1, PD-L1 and/or PD-L2. In embodiments, a lack of upregulation of one or more genes associated with positive regulation of IκB kinase/NFκB signaling compared to a standard indicates a response to the cancer therapy with an ability to inhibit function and/or activity of PD-1, PD-L1 and/or PD-L2.

In embodiments, an upregulation of one or more genes associated with regulation of innate immune response compared to a prior biological sample from the subject indicates resistance, a lack of response, or recalcitrance to the cancer therapy with an ability to inhibit function and/or activity of PD-1, PD-L1 and/or PD-L2. In embodiments, an upregulation of one or more genes associated with regulation of innate immune response compared to a patient that is known to be sensitive to anti-PD-1 therapy indicates resistance, a lack of response, or recalcitrance to the cancer therapy with an ability to inhibit function and/or activity of PD-1, PD-L1 and/or PD-L2. In embodiments, an upregulation of one or more genes associated with regulation of innate immune response compared to a standard indicates resistance, a lack of response, or recalcitrance to the cancer therapy with an ability to inhibit function and/or activity of PD-1, PD-L1 and/or PD-L2.

In embodiments, a lack of upregulation of one or more genes associated with regulation of innate immune response compared to a prior biological sample from the subject indicates a response to the cancer therapy with an ability to inhibit function and/or activity of PD-1, PD-L1 and/or PD-L2. In embodiments, a lack of upregulation of one or more genes associated with regulation of innate immune response compared to a patient that is known to be sensitive to anti-PD-1 therapy indicates a response to the cancer therapy with an ability to inhibit function and/or activity of PD-1, PD-L1 and/or PD-L2. In embodiments, a lack of upregulation of one or more genes associated with regulation of innate immune response compared to a standard indicates a response to the cancer therapy with an ability to inhibit function and/or activity of PD-1, PD-L1 and/or PD-L2.

In embodiments, an upregulation of one or more genes associated with negative regulation of antigen processing/presentation compared to a prior biological sample from the subject indicates resistance, a lack of response, or recalcitrance to the cancer therapy with an ability to inhibit function and/or activity of PD-1, PD-L1 and/or PD-L2. In embodiments, an upregulation of one or more genes associated with negative regulation of antigen processing/presentation compared to a patient that is known to be sensitive to anti-PD-1 therapy indicates resistance, a lack of response, or recalcitrance to the cancer therapy with an ability to inhibit function and/or activity of PD-1, PD-L1 and/or PD-L2. In embodiments, an upregulation of one or more genes associated with negative regulation of antigen processing/presentation compared to a standard indicates resistance, a lack of response, or recalcitrance to the cancer therapy with an ability to inhibit function and/or activity of PD-1, PD-L1 and/or PD-L2.

In embodiments, a lack of upregulation of one or more genes associated with negative regulation of antigen processing/presentation compared to a prior biological sample from the subject indicates a response to the cancer therapy with an ability to inhibit function and/or activity of PD-1, PD-L1 and/or PD-L2. In embodiments, a lack of upregulation of one or more genes associated with negative regulation of antigen processing/presentation compared to a patient that is known to be sensitive to anti-PD-1 therapy indicates a response to the cancer therapy with an ability to inhibit function and/or activity of PD-1, PD-L1 and/or PD-L2. In embodiments, a lack of upregulation of one or more genes associated with negative regulation of antigen processing/presentation compared to a standard indicates a response to the cancer therapy with an ability to inhibit function and/or activity of PD-1, PD-L1 and/or PD-L2.

In embodiments, an upregulation of one or more genes associated with antigen processing/presentation of endogenous peptides via MHC class I compared to a prior biological sample from the subject indicates resistance, a lack of response, or recalcitrance to the cancer therapy with an ability to inhibit function and/or activity of PD-1, PD-L1 and/or PD-L2. In embodiments, an upregulation of one or more genes associated with antigen processing/presentation of endogenous peptides via MHC class I compared to a patient that is known to be sensitive to anti-PD-1 therapy indicates resistance, a lack of response, or recalcitrance to the cancer therapy with an ability to inhibit function and/or activity of PD-1, PD-L1 and/or PD-L2. In embodiments, an upregulation of one or more genes associated with antigen processing/presentation of endogenous peptides via MHC class I compared to a standard indicates resistance, a lack of response, or recalcitrance to the cancer therapy with an ability to inhibit function and/or activity of PD-1, PD-L1 and/or PD-L2.

In embodiments, a lack of upregulation of one or more genes associated with antigen processing/presentation of endogenous peptides via MHC class I compared to a prior biological sample from the subject indicates a response to the cancer therapy with an ability to inhibit function and/or activity of PD-1, PD-L1 and/or PD-L2. In embodiments, a lack of upregulation of one or more genes associated with antigen processing/presentation of endogenous peptides via MHC class I compared to a patient that is known to be sensitive to anti-PD-1 therapy indicates a response to the cancer therapy with an ability to inhibit function and/or activity of PD-1, PD-L1 and/or PD-L2. In embodiments, a lack of upregulation of one or more genes associated with antigen processing/presentation of endogenous peptides via MHC class I compared to a standard indicates a response to the cancer therapy with an ability to inhibit function and/or activity of PD-1, PD-L1 and/or PD-L2.

In embodiments, an upregulation of one or more genes associated with positive regulation of IFNα production compared to a prior biological sample from the subject indicates resistance, a lack of response, or recalcitrance to the cancer therapy with an ability to inhibit function and/or activity of PD-1, PD-L1 and/or PD-L2. In embodiments, an upregulation of one or more genes associated with positive regulation of IFNα production compared to a patient that is known to be sensitive to anti-PD-1 therapy indicates resistance, a lack of response, or recalcitrance to the cancer therapy with an ability to inhibit function and/or activity of PD-1, PD-L1 and/or PD-L2. In embodiments, an upregulation of one or more genes associated with positive regulation of IFNα production compared to a standard indicates resistance, a lack of response, or recalcitrance to the cancer therapy with an ability to inhibit function and/or activity of PD-1, PD-L1 and/or PD-L2.

In embodiments, a lack of upregulation of one or more genes associated with positive regulation of IFNα production compared to a prior biological sample from the subject indicates a response to the cancer therapy with an ability to inhibit function and/or activity of PD-1, PD-L1 and/or PD-L2. In embodiments, a lack of upregulation of one or more genes associated with positive regulation of IFNα production compared to a patient that is known to be sensitive to anti-PD-1 therapy indicates a response to the cancer therapy with an ability to inhibit function and/or activity of PD-1, PD-L1 and/or PD-L2. In embodiments, a lack of upregulation of one or more genes associated with positive regulation of IFNα production compared to a standard indicates a response to the cancer therapy with an ability to inhibit function and/or activity of PD-1, PD-L1 and/or PD-L2.

In embodiments, an upregulation of one or more genes associated with positive regulation of defense response compared to a prior biological sample from the subject indicates resistance, a lack of response, or recalcitrance to the cancer therapy with an ability to inhibit function and/or activity of PD-1, PD-L1 and/or PD-L2. In embodiments, an upregulation of one or more genes associated with positive regulation of defense response compared to a patient that is known to be sensitive to anti-PD-1 therapy indicates resistance, a lack of response, or recalcitrance to the cancer therapy with an ability to inhibit function and/or activity of PD-1, PD-L1 and/or PD-L2. In embodiments, an upregulation of one or more genes associated with positive regulation of defense response compared to a standard indicates resistance, a lack of response, or recalcitrance to the cancer therapy with an ability to inhibit function and/or activity of PD-1, PD-L1 and/or PD-L2.

In embodiments, a lack of upregulation of one or more genes associated with positive regulation of defense response compared to a prior biological sample from the subject indicates a response to the cancer therapy with an ability to inhibit function and/or activity of PD-1, PD-L1 and/or PD-L2. In embodiments, a lack of upregulation of one or more genes associated with positive regulation of defense response compared to a patient that is known to be sensitive to anti-PD-1 therapy indicates a response to the cancer therapy with an ability to inhibit function and/or activity of PD-1, PD-L1 and/or PD-L2. In embodiments, a lack of upregulation of one or more genes associated with positive regulation of defense response compared to a standard indicates a response to the cancer therapy with an ability to inhibit function and/or activity of PD-1, PD-L1 and/or PD-L2.

In embodiments, an upregulation of one or more genes associated with positive regulation of IFNβ production compared to a prior biological sample from the subject indicates resistance, a lack of response, or recalcitrance to the cancer therapy with an ability to inhibit function and/or activity of PD-1, PD-L1 and/or PD-L2. In embodiments, an upregulation of one or more genes associated with positive regulation of IFNβ production compared to a patient that is known to be sensitive to anti-PD-1 therapy indicates resistance, a lack of response, or recalcitrance to the cancer therapy with an ability to inhibit function and/or activity of PD-1, PD-L1 and/or PD-L2. In embodiments, an upregulation of one or more genes associated with positive regulation of IFN production compared to a standard indicates resistance, a lack of response, or recalcitrance to the cancer therapy with an ability to inhibit function and/or activity of PD-1, PD-L1 and/or PD-L2.

In embodiments, a lack of upregulation of one or more genes associated with positive regulation of IFNβ production compared to a prior biological sample from the subject indicates a response to the cancer therapy with an ability to inhibit function and/or activity of PD-1, PD-L1 and/or PD-L2. In embodiments, a lack of upregulation of one or more genes associated with positive regulation of IFNβ production compared to a patient that is known to be sensitive to anti-PD-1 therapy indicates a response to the cancer therapy with an ability to inhibit function and/or activity of PD-1, PD-L1 and/or PD-L2. In embodiments, a lack of upregulation of one or more genes associated with positive regulation of IFNβ production compared to a standard indicates a response to the cancer therapy with an ability to inhibit function and/or activity of PD-1, PD-L1 and/or PD-L2.

In embodiments, an upregulation of one or more genes associated with regulation of inflammatory response compared to a prior biological sample from the subject indicates resistance, a lack of response, or recalcitrance to the cancer therapy with an ability to inhibit function and/or activity of PD-1, PD-L1 and/or PD-L2. In embodiments, an upregulation of one or more genes associated with regulation of inflammatory response compared to a patient that is known to be sensitive to anti-PD-1 therapy indicates resistance, a lack of response, or recalcitrance to the cancer therapy with an ability to inhibit function and/or activity of PD-1, PD-L1 and/or PD-L2. In embodiments, an upregulation of one or more genes associated with regulation of inflammatory response compared to a standard indicates resistance, a lack of response, or recalcitrance to the cancer therapy with an ability to inhibit function and/or activity of PD-1, PD-L1 and/or PD-L2.

In embodiments, a lack of upregulation of one or more genes associated with regulation of inflammatory response compared to a prior biological sample from the subject indicates a response to the cancer therapy with an ability to inhibit function and/or activity of PD-1, PD-L1 and/or PD-L2. In embodiments, a lack of upregulation of one or more genes associated with regulation of inflammatory response compared to a patient that is known to be sensitive to anti-PD-1 therapy indicates a response to the cancer therapy with an ability to inhibit function and/or activity of PD-1, PD-L1 and/or PD-L2. In embodiments, a lack of upregulation of one or more genes associated with regulation of inflammatory response compared to a standard indicates a response to the cancer therapy with an ability to inhibit function and/or activity of PD-1, PD-L1 and/or PD-L2.

In embodiments, a downregulation of one or more genes associated with phospholipid efflux compared to a prior biological sample from the subject indicates resistance, a lack of response, or recalcitrance to the cancer therapy with an ability to inhibit function and/or activity of PD-1, PD-L1 and/or PD-L2. In embodiments, a downregulation of one or more genes associated with phospholipid efflux compared to a patient that is known to be sensitive to anti-PD-1 therapy indicates resistance, a lack of response, or recalcitrance to the cancer therapy with an ability to inhibit function and/or activity of PD-1, PD-L1 and/or PD-L2. In embodiments, a downregulation of one or more genes associated with phospholipid efflux compared to a standard indicates resistance, a lack of response, or recalcitrance to the cancer therapy with an ability to inhibit function and/or activity of PD-1, PD-L1 and/or PD-L2.

In embodiments, a lack of downregulation of one or more genes associated with phospholipid efflux compared to a prior biological sample from the subject indicates a response to the cancer therapy with an ability to inhibit function and/or activity of PD-1, PD-L1 and/or PD-L2. In embodiments, a lack of downregulation of one or more genes associated with phospholipid efflux compared to a patient that is known to be sensitive to anti-PD-1 therapy indicates a response to the cancer therapy with an ability to inhibit function and/or activity of PD-1, PD-L1 and/or PD-L2. In embodiments, a lack of downregulation of one or more genes associated with phospholipid efflux compared to a standard indicates a response to the cancer therapy with an ability to inhibit function and/or activity of PD-1, PD-L1 and/or PD-L2.

In embodiments, a downregulation of one or more genes associated with negative regulation of fibrinolysis compared to a prior biological sample from the subject indicates resistance, a lack of response, or recalcitrance to the cancer therapy with an ability to inhibit function and/or activity of PD-1, PD-L1 and/or PD-L2. In embodiments, a downregulation of one or more genes associated with negative regulation of fibrinolysis compared to a patient that is known to be sensitive to anti-PD-1 therapy indicates resistance, a lack of response, or recalcitrance to the cancer therapy with an ability to inhibit function and/or activity of PD-1, PD-L1 and/or PD-L2. In embodiments, a downregulation of one or more genes associated with negative regulation of fibrinolysis compared to a standard indicates resistance, a lack of response, or recalcitrance to the cancer therapy with an ability to inhibit function and/or activity of PD-1, PD-L1 and/or PD-L2.

In embodiments, a lack of downregulation of one or more genes associated with negative regulation of fibrinolysis compared to a prior biological sample from the subject indicates a response to the cancer therapy with an ability to inhibit function and/or activity of PD-1, PD-L1 and/or PD-L2. In embodiments, a lack of downregulation of one or more genes associated with negative regulation of fibrinolysis compared to a patient that is known to be sensitive to anti-PD-1 therapy indicates a response to the cancer therapy with an ability to inhibit function and/or activity of PD-1, PD-L1 and/or PD-L2. In embodiments, a lack of downregulation of one or more genes associated with negative regulation of fibrinolysis compared to a standard indicates a response to the cancer therapy with an ability to inhibit function and/or activity of PD-1, PD-L1 and/or PD-L2.

In embodiments, a downregulation of one or more genes associated with chylomicron assembly compared to a prior biological sample from the subject indicates resistance, a lack of response, or recalcitrance to the cancer therapy with an ability to inhibit function and/or activity of PD-1, PD-L1 and/or PD-L2. In embodiments, a downregulation of one or more genes associated with chylomicron assembly compared to a patient that is known to be sensitive to anti-PD-1 therapy indicates resistance, a lack of response, or recalcitrance to the cancer therapy with an ability to inhibit function and/or activity of PD-1, PD-L1 and/or PD-L2. In embodiments, a downregulation of one or more genes associated with chylomicron assembly compared to a standard indicates resistance, a lack of response, or recalcitrance to the cancer therapy with an ability to inhibit function and/or activity of PD-1, PD-L1 and/or PD-L2.

In embodiments, a lack of downregulation of one or more genes associated with chylomicron assembly compared to a prior biological sample from the subject indicates a response to the cancer therapy with an ability to inhibit function and/or activity of PD-1, PD-L1 and/or PD-L2. In embodiments, a lack of downregulation of one or more genes associated with chylomicron assembly compared to a patient that is known to be sensitive to anti-PD-1 therapy indicates a response to the cancer therapy with an ability to inhibit function and/or activity of PD-1, PD-L1 and/or PD-L2. In embodiments, a lack of downregulation of one or more genes associated with chylomicron assembly compared to a standard indicates a response to the cancer therapy with an ability to inhibit function and/or activity of PD-1, PD-L1 and/or PD-L2.

In embodiments, a downregulation of one or more genes associated with plasma membrane repair compared to a prior biological sample from the subject indicates resistance, a lack of response, or recalcitrance to the cancer therapy with an ability to inhibit function and/or activity of PD-1, PD-L1 and/or PD-L2. In embodiments, a downregulation of one or more genes associated with plasma membrane repair compared to a patient that is known to be sensitive to anti-PD-1 therapy indicates resistance, a lack of response, or recalcitrance to the cancer therapy with an ability to inhibit function and/or activity of PD-1, PD-L1 and/or PD-L2. In embodiments, a downregulation of one or more genes associated with plasma membrane repair compared to a standard indicates resistance, a lack of response, or recalcitrance to the cancer therapy with an ability to inhibit function and/or activity of PD-1, PD-L1 and/or PD-L2.

In embodiments, a lack of downregulation of one or more genes associated with plasma membrane repair compared to a prior biological sample from the subject indicates a response to the cancer therapy with an ability to inhibit function and/or activity of PD-1, PD-L1 and/or PD-L2. In embodiments, a lack of downregulation of one or more genes associated with plasma membrane repair compared to a patient that is known to be sensitive to anti-PD-1 therapy indicates a response to the cancer therapy with an ability to inhibit function and/or activity of PD-1, PD-L1 and/or PD-L2. In embodiments, a lack of downregulation of one or more genes associated with plasma membrane repair compared to a standard indicates a response to the cancer therapy with an ability to inhibit function and/or activity of PD-1, PD-L1 and/or PD-L2.

In embodiments, a downregulation of one or more genes associated with SRP-dependent co-translational protein targeting to membrane compared to a prior biological sample from the subject indicates resistance, a lack of response, or recalcitrance to the cancer therapy with an ability to inhibit function and/or activity of PD-1, PD-L1 and/or PD-L2. In embodiments, a downregulation of one or more genes associated with SRP-dependent co-translational protein targeting to membrane compared to a patient that is known to be sensitive to anti-PD-1 therapy indicates resistance, a lack of response, or recalcitrance to the cancer therapy with an ability to inhibit function and/or activity of PD-1, PD-L1 and/or PD-L2. In embodiments, a downregulation of one or more genes associated with SRP-dependent co-translational protein targeting to membrane compared to a standard indicates resistance, a lack of response, or recalcitrance to the cancer therapy with an ability to inhibit function and/or activity of PD-1, PD-L1 and/or PD-L2.

In embodiments, a lack of downregulation of one or more genes associated with SRP-dependent co-translational protein targeting to membrane compared to a prior biological sample from the subject indicates a response to the cancer therapy with an ability to inhibit function and/or activity of PD-1, PD-L1 and/or PD-L2. In embodiments, a lack of downregulation of one or more genes associated with SRP-dependent co-translational protein targeting to membrane compared to a patient that is known to be sensitive to anti-PD-1 therapy indicates a response to the cancer therapy with an ability to inhibit function and/or activity of PD-1, PD-L1 and/or PD-L2. In embodiments, a lack of downregulation of one or more genes associated with SRP-dependent co-translational protein targeting to membrane compared to a standard indicates a response to the cancer therapy with an ability to inhibit function and/or activity of PD-1, PD-L1 and/or PD-L2.

In embodiments, a downregulation of one or more genes associated with ribosomal small subunit assembly compared to a prior biological sample from the subject indicates resistance, a lack of response, or recalcitrance to the cancer therapy with an ability to inhibit function and/or activity of PD-1, PD-L1 and/or PD-L2. In embodiments, a downregulation of one or more genes associated with ribosomal small subunit assembly compared to a patient that is known to be sensitive to anti-PD-1 therapy indicates resistance, a lack of response, or recalcitrance to the cancer therapy with an ability to inhibit function and/or activity of PD-1, PD-L1 and/or PD-L2. In embodiments, a downregulation of one or more genes associated with ribosomal small subunit assembly compared to a standard indicates resistance, a lack of response, or recalcitrance to the cancer therapy with an ability to inhibit function and/or activity of PD-1, PD-L1 and/or PD-L2.

In embodiments, a lack of downregulation of one or more genes associated with ribosomal small subunit assembly compared to a prior biological sample from the subject indicates a response to the cancer therapy with an ability to inhibit function and/or activity of PD-1, PD-L1 and/or PD-L2. In embodiments, a lack of downregulation of one or more genes associated with ribosomal small subunit assembly compared to a patient that is known to be sensitive to anti-PD-1 therapy indicates a response to the cancer therapy with an ability to inhibit function and/or activity of PD-1, PD-L1 and/or PD-L2. In embodiments, a lack of downregulation of one or more genes associated with ribosomal small subunit assembly compared to a standard indicates a response to the cancer therapy with an ability to inhibit function and/or activity of PD-1, PD-L1 and/or PD-L2.

In embodiments, a downregulation of one or more genes associated with phospholipid efflux compared to a prior biological sample from the subject indicates resistance, a lack of response, or recalcitrance to the cancer therapy with an ability to inhibit function and/or activity of PD-1, PD-L1 and/or PD-L2. In embodiments, a downregulation of one or more genes associated with phospholipid efflux compared to a patient that is known to be sensitive to anti-PD-1 therapy indicates resistance, a lack of response, or recalcitrance to the cancer therapy with an ability to inhibit function and/or activity of PD-1, PD-L1 and/or PD-L2. In embodiments, a downregulation of one or more genes associated with phospholipid efflux compared to a standard indicates resistance, a lack of response, or recalcitrance to the cancer therapy with an ability to inhibit function and/or activity of PD-1, PD-L1 and/or PD-L2.

In embodiments, a lack of downregulation of one or more genes associated with phospholipid efflux compared to a prior biological sample from the subject indicates a response to the cancer therapy with an ability to inhibit function and/or activity of PD-1, PD-L1 and/or PD-L2. In embodiments, a lack of downregulation of one or more genes associated with phospholipid efflux compared to a patient that is known to be sensitive to anti-PD-1 therapy indicates a response to the cancer therapy with an ability to inhibit function and/or activity of PD-1, PD-L1 and/or PD-L2. In embodiments, a lack of downregulation of one or more genes associated with phospholipid efflux compared to a standard indicates a response to the cancer therapy with an ability to inhibit function and/or activity of PD-1, PD-L1 and/or PD-L2.

In embodiments, a downregulation of one or more genes associated with regulation of translation compared to a prior biological sample from the subject indicates resistance, a lack of response, or recalcitrance to the cancer therapy with an ability to inhibit function and/or activity of PD-1, PD-L1 and/or PD-L2. In embodiments, a downregulation of one or more genes associated with regulation of translation compared to a patient that is known to be sensitive to anti-PD-1 therapy indicates resistance, a lack of response, or recalcitrance to the cancer therapy with an ability to inhibit function and/or activity of PD-1, PD-L1 and/or PD-L2. In embodiments, a downregulation of one or more genes associated with regulation of translation compared to a standard indicates resistance, a lack of response, or recalcitrance to the cancer therapy with an ability to inhibit function and/or activity of PD-1, PD-L1 and/or PD-L2.

In embodiments, a lack of downregulation of one or more genes associated with regulation of translation compared to a prior biological sample from the subject indicates a response to the cancer therapy with an ability to inhibit function and/or activity of PD-1, PD-L1 and/or PD-L2. In embodiments, a lack of downregulation of one or more genes associated with regulation of translation compared to a patient that is known to be sensitive to anti-PD-1 therapy indicates a response to the cancer therapy with an ability to inhibit function and/or activity of PD-1, PD-L1 and/or PD-L2. In embodiments, a lack of downregulation of one or more genes associated with regulation of translation compared to a standard indicates a response to the cancer therapy with an ability to inhibit function and/or activity of PD-1, PD-L1 and/or PD-L2.

In embodiments, a downregulation of one or more genes associated with mitochondrial respiratory chain complex I compared to a prior biological sample from the subject indicates resistance, a lack of response, or recalcitrance to the cancer therapy with an ability to inhibit function and/or activity of PD-1, PD-L1 and/or PD-L2. In embodiments, a downregulation of one or more genes associated with mitochondrial respiratory chain complex I compared to a patient that is known to be sensitive to anti-PD-1 therapy indicates resistance, a lack of response, or recalcitrance to the cancer therapy with an ability to inhibit function and/or activity of PD-1, PD-L1 and/or PD-L2. In embodiments, a downregulation of one or more genes associated with mitochondrial respiratory chain complex I compared to a standard indicates resistance, a lack of response, or recalcitrance to the cancer therapy with an ability to inhibit function and/or activity of PD-1, PD-L1 and/or PD-L2.

In embodiments, a lack of downregulation of one or more genes associated with mitochondrial respiratory chain complex I compared to a prior biological sample from the subject indicates a response to the cancer therapy with an ability to inhibit function and/or activity of PD-1, PD-L1 and/or PD-L2. In embodiments, a lack of downregulation of one or more genes associated with mitochondrial respiratory chain complex I compared to a patient that is known to be sensitive to anti-PD-1 therapy indicates a response to the cancer therapy with an ability to inhibit function and/or activity of PD-1, PD-L1 and/or PD-L2. In embodiments, a lack of downregulation of one or more genes associated with mitochondrial respiratory chain complex I compared to a standard indicates a response to the cancer therapy with an ability to inhibit function and/or activity of PD-1, PD-L1 and/or PD-L2.

In embodiments, a downregulation of one or more genes associated with mitochondrial translational elongation compared to a prior biological sample from the subject indicates resistance, a lack of response, or recalcitrance to the cancer therapy with an ability to inhibit function and/or activity of PD-1, PD-L1 and/or PD-L2. In embodiments, a downregulation of one or more genes associated with mitochondrial translational elongation compared to a patient that is known to be sensitive to anti-PD-1 therapy indicates resistance, a lack of response, or recalcitrance to the cancer therapy with an ability to inhibit function and/or activity of PD-1, PD-L1 and/or PD-L2. In embodiments, a downregulation of one or more genes associated with mitochondrial translational elongation compared to a standard indicates resistance, a lack of response, or recalcitrance to the cancer therapy with an ability to inhibit function and/or activity of PD-1, PD-L1 and/or PD-L2.

In embodiments, a lack of downregulation of one or more genes associated with mitochondrial translational elongation compared to a prior biological sample from the subject indicates a response to the cancer therapy with an ability to inhibit function and/or activity of PD-1, PD-L1 and/or PD-L2. In embodiments, a lack of downregulation of one or more genes associated with mitochondrial translational elongation compared to a patient that is known to be sensitive to anti-PD-1 therapy indicates a response to the cancer therapy with an ability to inhibit function and/or activity of PD-1, PD-L1 and/or PD-L2. In embodiments, a lack of downregulation of one or more genes associated with mitochondrial translational elongation compared to a standard indicates a response to the cancer therapy with an ability to inhibit function and/or activity of PD-1, PD-L1 and/or PD-L2.

In embodiments, a downregulation of one or more genes associated with DNA-dependent DNA replication compared to a prior biological sample from the subject indicates resistance, a lack of response, or recalcitrance to the cancer therapy with an ability to inhibit function and/or activity of PD-1, PD-L1 and/or PD-L2. In embodiments, a downregulation of one or more genes associated with DNA-dependent DNA replication compared to a patient that is known to be sensitive to anti-PD-1 therapy indicates resistance, a lack of response, or recalcitrance to the cancer therapy with an ability to inhibit function and/or activity of PD-1, PD-L1 and/or PD-L2. In embodiments, a downregulation of one or more genes associated with DNA-dependent DNA replication compared to a standard indicates resistance, a lack of response, or recalcitrance to the cancer therapy with an ability to inhibit function and/or activity of PD-1, PD-L1 and/or PD-L2.

In embodiments, a lack of downregulation of one or more genes associated with DNA-dependent DNA replication compared to a prior biological sample from the subject indicates a response to the cancer therapy with an ability to inhibit function and/or activity of PD-1, PD-L1 and/or PD-L2. In embodiments, a lack of downregulation of one or more genes associated with DNA-dependent DNA replication compared to a patient that is known to be sensitive to anti-PD-1 therapy indicates a response to the cancer therapy with an ability to inhibit function and/or activity of PD-1, PD-L1 and/or PD-L2. In embodiments, a lack of downregulation of one or more genes associated with DNA-dependent DNA replication compared to a standard indicates a response to the cancer therapy with an ability to inhibit function and/or activity of PD-1, PD-L1 and/or PD-L2.

In embodiments, a downregulation of one or more genes associated with ATP biosynthetic process compared to a prior biological sample from the subject indicates resistance, a lack of response, or recalcitrance to the cancer therapy with an ability to inhibit function and/or activity of PD-1, PD-L1 and/or PD-L2. In embodiments, a downregulation of one or more genes associated with ATP biosynthetic process compared to a patient that is known to be sensitive to anti-PD-1 therapy indicates resistance, a lack of response, or recalcitrance to the cancer therapy with an ability to inhibit function and/or activity of PD-1, PD-L1 and/or PD-L2. In embodiments, a downregulation of one or more genes associated with ATP biosynthetic process compared to a standard indicates resistance, a lack of response, or recalcitrance to the cancer therapy with an ability to inhibit function and/or activity of PD-1, PD-L1 and/or PD-L2.

In embodiments, a lack of downregulation of one or more genes associated with ATP biosynthetic process compared to a prior biological sample from the subject indicates a response to the cancer therapy with an ability to inhibit function and/or activity of PD-1, PD-L1 and/or PD-L2. In embodiments, a lack of downregulation of one or more genes associated with ATP biosynthetic process compared to a patient that is known to be sensitive to anti-PD-1 therapy indicates a response to the cancer therapy with an ability to inhibit function and/or activity of PD-1, PD-L1 and/or PD-L2. In embodiments, a lack of downregulation of one or more genes associated with ATP biosynthetic process compared to a standard indicates a response to the cancer therapy with an ability to inhibit function and/or activity of PD-1, PD-L1 and/or PD-L2.

In embodiments, a cancer therapy with an ability to inhibit function and/or activity of PD-1, PD-L1 and/or PD-L2 may be indicated, for example, when expression of one or more of Rpl41, Rps15 and Rps8 is high and/or the expression of one or more of Cd274, B2M, Tap1, Tap2, Casp1, and Gasta3 is low. In embodiments, a cancer therapy with an ability to inhibit function and/or activity of PD-1, PD-L1 and/or PD-L2 may not be indicated, for example, when expression of one or more of Rpl41, Rps15 and Rps8 is low and/or the expression of one or more of Cd274, B2M, Tap1, Tap2, Casp1, and Gasta3 is high. In embodiments, a patient featuring low expression of one or more of Rpl41, Rps15 and Rps8 and/or high expression of one or more of Cd274, B2M, Tap1, Tap2, Casp1, and Gasta3 are likely to benefit from adjuvant or neoadjuvant therapies that eliminate the PD-1-nonresponsive cells.

In embodiments, the biological sample is a fresh tissue sample, frozen tumor tissue specimen, cultured cells, circulating tumor cells, or a formalin-fixed paraffin-embedded tumor tissue specimen. In embodiments, the biological sample is a biopsy sample. In embodiments, the biopsy sample is selected from endoscopic biopsy, bone marrow biopsy, endoscopic biopsy (e.g., cystoscopy, bronchoscopy and colonoscopy), needle biopsy (e.g., fine-needle aspiration, core needle biopsy, vacuum-assisted biopsy, X-ray-assisted biopsy, computerized tomography (CT)-assisted biopsy, magnetic resonance imaging (MRI)-assisted biopsy and ultrasound-assisted biopsy), skin biopsy (e.g., shave biopsy, punch biopsy, and incisional biopsy) and surgical biopsy.

In embodiments, the biological sample comprises a body fluid selected from blood, plasma, serum, lacrimal fluid, tears, bone marrow, blood, blood cells, ascites, tissue or fine needle biopsy sample, cell-containing body fluid, free floating nucleic acids, sputum, saliva, urine, cerebrospinal fluid, peritoneal fluid, pleural fluid, feces, lymph, gynecological fluid, skin swab, vaginal swab, oral swab, nasal swab, washing or lavage such as a ductal lavage or broncheoalveolar lavage, aspirate, scraping, bone marrow specimen, tissue biopsy specimen, surgical specimen, feces, other body fluids, secretions, and/or excretions, and/or cells therefrom. In embodiments, the biological sample is obtained by a technique selected from scrapes, swabs, and biopsy. In embodiments, the biological sample is obtained by use of brushes, (cotton) swabs, spatula, rinse/wash fluids, punch biopsy devices, puncture of cavities with needles or surgical instrumentation.

In embodiments, the biological sample comprises at least one tumor cell. In embodiments, the tumor is selected from Hodgkin's and non-Hodgkin's lymphoma, B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; or chronic myeloblastic leukemia. In some embodiments, the cancer is basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (e.g., that associated with brain tumors), Meigs' syndrome cancer; renal carcinoma; colorectal cancer; and adrenal cancer.

In embodiments, the evaluating is performed by DNA sequencing, RNA sequencing, immunohistochemical staining, western blotting, in cell western, immunofluorescent staining, ELISA, and fluorescent activating cell sorting (FACS) or a combination thereof.

In embodiments, the evaluating is performed by contacting the sample with an agent that specifically binds to one or more proteins encoded by one or more genes associated with a gene ontology (GO) pathway selected from: (i) positive regulation of cell cycle process, regulation of G1/S transition, regulation of cell division, regulation of cell proliferation, positive regulation of IκB kinase/NFκB signaling, type I IFN signaling pathway, cellular response to IFNγ, positive regulation of IFNα production, positive regulation of defense response, positive regulation of IFNβ production, regulation of inflammatory response, regulation of innate immune response, negative regulation of antigen processing and presentation, and antigen processing, presentation of endogenous peptides via MHC class I; and/or (ii) phospholipid efflux, negative regulation of fibrinolysis, chylomicron assembly, plasma membrane repair, SRP-dependent co-translational protein targeting to membrane, ribosomal small subunit assembly, phospholipid efflux, regulation of translation, mitochondrial respiratory chain complex I, mitochondrial translational elongation, DNA-dependent DNA replication, and ATP biosynthetic process.

In embodiments, the evaluating is performed by contacting the sample with an agent that specifically binds to one or more proteins encoded by one or more genes associated with a gene ontology (GO) pathway selected from (a) cellular response to IFNγ, (b) negative regulation of antigen processing and presentation, (c) type I IFN signaling pathway, (d) positive regulation of IκB kinase/NFκB signaling, and antigen processing, and (e) presentation of endogenous peptides via MHC class I. In embodiments, the evaluating is performed by contacting the sample with an agent that specifically binds to one or more proteins encoded by one or more genes associated with cellular response to IFNγ. In embodiments, the evaluating is performed by contacting the sample with an agent that specifically binds to one or more proteins encoded by one or more genes associated with type I IFN signaling pathway. In embodiments, the evaluating is performed by contacting the sample with an agent that specifically binds to one or more proteins encoded by one or more genes associated with positive regulation of IFNα production. In embodiments, the evaluating is performed by contacting the sample with an agent that specifically binds to one or more proteins encoded by one or more genes associated with positive regulation of defense response. In embodiments, the evaluating is performed by contacting the sample with an agent that specifically binds to one or more proteins encoded by one or more genes associated with positive regulation of IFNβ production. In embodiments, the evaluating is performed by contacting the sample with an agent that specifically binds to one or more proteins encoded by one or more genes associated with regulation of inflammatory response.

In embodiments, the evaluating is performed by contacting the sample with an agent that specifically binds to one or more of nucleic acids of one or more genes associated with a gene ontology (GO) pathway selected from: (i) positive regulation of cell cycle process, regulation of G1/S transition, regulation of cell division, regulation of cell proliferation, positive regulation of IκB kinase/NFκB signaling, type I IFN signaling pathway, cellular response to IFNγ, positive regulation of IFNα production, positive regulation of defense response, positive regulation of IFNβ production, regulation of inflammatory response, regulation of innate immune response, negative regulation of antigen processing and presentation, and antigen processing, presentation of endogenous peptides via MHC class I; and/or (ii) phospholipid efflux, negative regulation of fibrinolysis, chylomicron assembly, plasma membrane repair, SRP-dependent co-translational protein targeting to membrane, ribosomal small subunit assembly, phospholipid efflux, regulation of translation, mitochondrial respiratory chain complex I, mitochondrial translational elongation, DNA-dependent DNA replication, and ATP biosynthetic process.

In embodiments, the evaluating is performed by contacting the sample with an agent that specifically binds to one or more nucleic acid of one or more genes associated with a gene ontology (GO) pathway selected from (a) cellular response to IFNγ, (b) negative regulation of antigen processing and presentation, (c) type I IFN signaling pathway, (d) positive regulation of IκB kinase/NFκB signaling, and antigen processing, and (e) presentation of endogenous peptides via MHC class I. In embodiments, the evaluating is performed by contacting the sample with an agent that specifically binds to one or more nucleic acid of one or more genes associated with cellular response to IFNγ. In embodiments, the evaluating is performed by contacting the sample with an agent that specifically binds to one or more nucleic acid of one or more genes associated with type I IFN signaling pathway. In embodiments, the evaluating is performed by contacting the sample with an agent that specifically binds to one or more nucleic acid of one or more genes associated with positive regulation of IFNα production. In embodiments, the evaluating is performed by contacting the sample with an agent that specifically binds to one or more nucleic acid of one or more genes associated with positive regulation of defense response. In embodiments, the evaluating is performed by contacting the sample with an agent that specifically binds to one or more nucleic acid of one or more genes associated with positive regulation of IFN production. In embodiments, the evaluating is performed by contacting the sample with an agent that specifically binds to one or more nucleic acid of one or more genes associated with regulation of inflammatory response. In embodiments, the agent that specifically binds to one or more of the nucleic acids is a nucleic acid primer or probe.

In embodiments, the evaluating informs classifying the patient into a high or low risk group. In embodiments, the high risk classification comprises a high level of tumor cells having resistance to the cancer therapy with an ability to inhibit function and/or activity of PD-1, PD-L1 and/or PD-L2. In embodiments, the low risk classification comprises a low level of tumor cells having resistance to the cancer therapy with an ability to inhibit function and/or activity of PD-1, PD-L1 and/or PD-L2.

In embodiments, the low risk or high risk classification is indicative of withholding of the neoadjuvant therapy. In embodiments, the low risk or high risk classification is indicative of withholding of the adjuvant therapy. In embodiments, the evaluating is predictive of a positive response to and/or benefit from the cancer treatment. In embodiments, the evaluating is predictive of a negative or neutral response to and/or benefit from the cancer treatment. In embodiments, the evaluating is predictive of a positive response to and/or benefit from neoadjuvant chemotherapy or a non-responsiveness to and/or lack of benefit from neoadjuvant chemotherapy In embodiments, the evaluating is predictive of a positive response to and/or benefit from adjuvant chemotherapy or a non-responsiveness to and/or lack of benefit from adjuvant chemotherapy. In embodiments, the evaluating is predictive of a negative or neutral response to and/or benefit from neoadjuvant chemotherapy or a non-responsiveness to and/or lack of benefit from neoadjuvant chemotherapy. In embodiments, the evaluating is predictive of a negative or neutral response to and/or benefit from adjuvant chemotherapy or a non-responsiveness to and/or lack of benefit from adjuvant chemotherapy. In embodiments, the evaluating informs administration or withholding of the cancer treatment. In embodiments, the evaluating informs administration of neoadjuvant therapy. In embodiments, the evaluating informs administration of adjuvant therapy. In embodiments, the evaluating informs withholding of neoadjuvant therapy. In embodiments, the evaluating informs withholding of adjuvant therapy. In embodiments, the neoadjuvant therapy and/or the adjuvant therapy is a chemotherapeutic agent. In embodiments, the neoadjuvant therapy and/or the adjuvant therapy is a cytotoxic agent. In embodiments, the neoadjuvant therapy and/or the adjuvant therapy is a checkpoint inhibitor.

In embodiments, the anti-cancer drug candidate is selected from a chemotherapeutic agent, a cytotoxic agent, and a checkpoint inhibitor.

In embodiments, the classes of chemotherapy include antimetabolite chemotherapeutics (e.g., 5-fluorouracil, methotrexate, capecitabine, azacitidine, etc.) or topoisomerase inhibitors (e.g., irinotecan, topotecan, etoposide, doxorubicin, etc.)

In embodiments, the chemotherapy is selected from protein translation (e.g., assembly and/or function of ribosomal complex, adequate expression and/or function of tRNA, adequate synthesis and/or uptake of amino acids, etc.), post-translational modification (e.g., decoration of the translated protein with carbohydrates important for function or protection from degradation), or transport mechanisms (e.g., post-translational peptide processing, signal peptide recognition and cleavage, transport through the ER/Golgi network, etc.) may be helpful as combining defects in post-translational processes to create synthetic lethal phenotypes in cancer cell populations developing resistance to PD-1 or PD-L1 blocking agents. the classes of chemotherapy include antimetabolite chemotherapeutics (e.g., 5-fluorouracil, methotrexate, capecitabine, azacitidine, etc.) or topoisomerase inhibitors (e.g., irinotecan, topotecan, etoposide, doxorubicin, etc.). in embodiments, the neoadjuvant therapy and/or the adjuvant therapy is selected from a chemotherapeutic agent, a cytotoxic agent, a checkpoint inhibitor, an antimetabolite chemotherapeutic (e.g., 5-fluorouracil, methotrexate, capecitabine, azacitidine), a topoisomerase inhibitor (e.g., irinotecan, topotecan, etoposide, doxorubicin, etc.). In embodiments, the neoadjuvant therapy and/or the adjuvant therapy is selected from a protein translation inhibitor (e.g., a modulator of assembly and/or function of ribosomal complex, a modulator of expression and/or function of tRNA, a modulator of synthesis and/or uptake of amino acids, a modulator of post-translational modification (e.g., decoration of the translated protein with carbohydrates), a modulator of protein degradation, and a modulator of protein transport (e.g., post-translational peptide processing, signal peptide recognition and cleavage, transport through the ER/Golgi network, etc.), etc.) or a topoisomerase inhibitor.

In embodiments, the neoadjuvant therapy and/or the adjuvant therapy is selected from a protein translation inhibitor (e.g., silvestrol and omacetaxine) ribosome biogenesis inhibitors (e.g., diazaborine, lamotrigine and ribozinoindoles), inhibitors of rRNA and/or tRNA synthesis (e.g., quarfloxin (CX-3543) and CX-5461), an inhibitor of synthesis of amino acids (e.g., GLUD1 inhibitor R162, BCAT1 inhibitor gabapentin, glutaminase inhibitor bis-2-(5-phenylacetamido-1,2,4-thiadiazol-2-yl)ethyl sulfide (BPTES), PAGDH inhibitor NCT-503), an inhibitor of uptake of amino acids (e.g., SLC7A11 inhibitors sulfasalazine, erastin or sorafenib), a modulator of post-translational modification (e.g., glycosylation inhibitor tunicamycin, ppGalNAc-T3), a modulator of protein degradation, and a modulator of protein transport (e.g., cyclosporin A, fendiline, parbendazole, paroxetine, parthenolide, quinacrine, sertraline, spiperone, thimerosal, astemizole, perhexiline, HUN-7293, CAM741, CK147, and cotransin).

In embodiments, the upregulation is in comparison to a healthy tissue. In embodiments, the upregulation is in comparison to another biological sample from patient that is known to be sensitive to anti-PD-1 therapy. In embodiments, the upregulation is in comparison to a prior biological sample obtained from the subject. In embodiments, the downregulation is in comparison to a healthy tissue. In embodiments, the downregulation is in comparison to another biological sample from patient that is known to be sensitive to anti-PD-1 therapy. In embodiments, the downregulation is in comparison to a prior biological sample obtained from the subject.

In embodiments, the biological sample is a fresh tissue sample, frozen tumor tissue specimen, cultured cells, circulating tumor cells, or a formalin-fixed paraffin-embedded tumor tissue specimen. In embodiments, the biological sample is a biopsy sample. In embodiments, the biopsy sample is selected from endoscopic biopsy, bone marrow biopsy, endoscopic biopsy (e.g., cystoscopy, bronchoscopy and colonoscopy), needle biopsy (e.g., fine-needle aspiration, core needle biopsy, vacuum-assisted biopsy, X-ray-assisted biopsy, computerized tomography (CT)-assisted biopsy, magnetic resonance imaging (MRI)-assisted biopsy and ultrasound-assisted biopsy), skin biopsy (e.g., shave biopsy, punch biopsy, and incisional biopsy) and surgical biopsy. In embodiments, the biological sample comprises a body fluid selected from blood, plasma, serum, lacrimal fluid, tears, bone marrow, blood, blood cells, ascites, tissue or fine needle biopsy sample, cell-containing body fluid, free floating nucleic acids, sputum, saliva, urine, cerebrospinal fluid, peritoneal fluid, pleural fluid, feces, lymph, gynecological fluid, skin swab, vaginal swab, oral swab, nasal swab, washing or lavage such as a ductal lavage or broncheoalveolar lavage, aspirate, scraping, bone marrow specimen, tissue biopsy specimen, surgical specimen, feces, other body fluids, secretions, and/or excretions, and/or cells therefrom. In embodiments, the biological sample is obtained by a technique selected from scrapes, swabs, and biopsy. In embodiments, the biological sample is obtained by use of brushes, (cotton) swabs, spatula, rinse/wash fluids, punch biopsy devices, puncture of cavities with needles or surgical instrumentation. In embodiments, the biological sample comprises at least one tumor cell.

In embodiments, the tumor is selected from Hodgkin's and non-Hodgkin's lymphoma, B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; or chronic myeloblastic leukemia, basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (e.g., that associated with brain tumors), Meigs' syndrome cancer; renal carcinoma; colorectal cancer; and adrenal cancer.

In embodiments, the evaluating is performed by DNA sequencing, RNA sequencing, immunohistochemical staining, western blotting, in cell western, immunofluorescent staining, ELISA, and fluorescent activating cell sorting (FACS) or a combination thereof. In embodiments, the evaluating is performed by contacting the sample with an agent that specifically binds to one or more proteins encoded by one or more genes associated with a gene ontology (GO) pathway selected from: (i) positive regulation of cell cycle process, regulation of G1/S transition, regulation of cell division, regulation of cell proliferation, positive regulation of IκB kinase/NFκB signaling, type I IFN signaling pathway, cellular response to IFNγ, positive regulation of IFNα production, positive regulation of defense response, positive regulation of IFNβ production, regulation of inflammatory response, regulation of innate immune response, negative regulation of antigen processing/presentation, and antigen processing/presentation of endogenous peptides via MHC class I; and/or (ii) phospholipid efflux, negative regulation of fibrinolysis, chylomicron assembly, plasma membrane repair, SRP-dependent co-translational protein targeting to membrane, ribosomal small subunit assembly, phospholipid efflux, regulation of translation, mitochondrial respiratory chain complex I, mitochondrial translational elongation, DNA-dependent DNA replication, and ATP biosynthetic process.

In embodiments, the evaluating is performed by contacting the sample with an agent that specifically binds to one or more proteins encoded by one or more genes associated with a gene ontology (GO) pathway selected from (a) cellular response to IFNγ, (b) negative regulation of antigen processing/presentation, (c) type I IFN signaling pathway, (d) positive regulation of IκB kinase/NFκB signaling, and antigen processing, and (e) presentation of endogenous peptides via MHC class I. In embodiments, the evaluating is performed by contacting the sample with an agent that specifically binds to one or more proteins encoded by one or more genes associated with cellular response to IFNγ. In embodiments, the evaluating is performed by contacting the sample with an agent that specifically binds to one or more proteins encoded by one or more genes associated with type I IFN signaling pathway.

In embodiments, the evaluating is performed by contacting the sample with an agent that specifically binds to one or more of nucleic acids of one or more genes associated with a gene ontology (GO) pathway selected from: (i) positive regulation of cell cycle process, regulation of G1/S transition, regulation of cell division, regulation of cell proliferation, positive regulation of IκB kinase/NFκB signaling, type I IFN signaling pathway, cellular response to IFNγ, positive regulation of IFNα production, positive regulation of defense response, positive regulation of IFNβ production, regulation of inflammatory response, regulation of innate immune response, negative regulation of antigen processing/presentation, and antigen processing/presentation of endogenous peptides via MHC class I; and/or (ii) phospholipid efflux, negative regulation of fibrinolysis, chylomicron assembly, plasma membrane repair, SRP-dependent co-translational protein targeting to membrane, ribosomal small subunit assembly, phospholipid efflux, regulation of translation, mitochondrial respiratory chain complex I, mitochondrial translational elongation, DNA-dependent DNA replication, and ATP biosynthetic process. In embodiments, the evaluating is performed by contacting the sample with an agent that specifically binds to one or more nucleic acid of one or more genes associated with a gene ontology (GO) pathway selected from (a) cellular response to IFNγ, (b) negative regulation of antigen processing/presentation, (c) type I IFN signaling pathway, (d) positive regulation of IκB kinase/NFκB signaling, and antigen processing, and (e) presentation of endogenous peptides via MHC class I. In embodiments, the evaluating is performed by contacting the sample with an agent that specifically binds to one or more nucleic acid of one or more genes associated with cellular response to IFNγ. In embodiments, the evaluating is performed by contacting the sample with an agent that specifically binds to one or more nucleic acid of one or more genes associated with type I IFN signaling pathway. In embodiments, the agent that specifically binds to one or more of the nucleic acids is a nucleic acid primer or probe.

In embodiments, the evaluating informs classifying the patient into a high or low risk group. In embodiments, the high risk classification comprises a high level of tumor cells having resistance to the cancer therapy with an ability to inhibit function and/or activity of PD-1, PD-L1 and/or PD-L2. In embodiments, the low risk classification comprises a low level of tumor cells having resistance to the cancer therapy with an ability to inhibit function and/or activity of PD-1, PD-L1 and/or PD-L2. In embodiments, the low risk classification is indicative of withholding of the cancer therapy. In embodiments, the high risk classification is indicative of administering the cancer therapy.

In one aspect, the present disclosure relates to a method of determining a cancer treatment for a patient, the method comprising: (a) obtaining a biological sample from a subject; (b) evaluating the biological sample for the expression of: (i) a gene selected from CD274, B2M, STAT1, STAT2, TRIM7, IRF1, TAP1, TAP2, CASP1, IRF, LTBR, PVR, GASTA3, LRG1, SPRY2, ARG1, TRIM8, TRIM2, MAPK81P1, TRIM6, and KRT1; and/or (ii) a gene selected from RPL41, RPS15, RPS8, TRIM7 and LRG1; and (c) selecting the cancer therapy based on the evaluation of step (b).

In one aspect, the present disclosure relates to a method of determining a cancer treatment for a patient, the method comprising: (a) obtaining a biological sample from a subject; (b) evaluating the biological sample for the expression of: (i) a gene selected from CD274, B2M, STAT1, STAT2, TRIM7, IRF1, TAP1, TAP2, CASP1, IRF, LTBR, PVR, GASTA3, LRG1, SPRY2, ARG1, TRIM8, TRIM2, MAPK81P1, TRIM6, and KRT1; and/or (ii) a gene selected from RPL41, RPS15, RPS8, TRIM7 and LRG1; and (c) selecting the cancer therapy based on the evaluation of step (b), wherein cancer therapy is selected from: (i) an agent with an ability to inhibit function and/or activity of PD-1, PD-L1 and/or PD-L2; (ii) an antimetabolite chemotherapeutic (e.g., 5-fluorouracil, methotrexate, capecitabine, azacitidine, 6-diazo-5-oxo-L-norleucine (DON), azaserine and acivicin), a topoisomerase inhibitor (e.g., irinotecan, topotecan, etoposide, doxorubicin, etc.); and (iii) a protein translation inhibitor (e.g., silvestrol and omacetaxine) ribosome biogenesis inhibitors (e.g., diazaborine, lamotrigine and ribozinoindoles), inhibitors of rRNA and/or tRNA synthesis (e.g., quarfloxin (CX-3543) and CX-5461), an inhibitor of synthesis of amino acids (e.g., GLUD1 inhibitor R162, BCAT1 inhibitor gabapentin, glutaminase inhibitor bis-2-(5-phenylacetamido-1,2,4-thiadiazol-2-yl)ethyl sulfide (BPTES), PAGDH inhibitor NCT-503), an inhibitor of uptake of amino acids (e.g., SLC7A11 inhibitors sulfasalazine, erastin or sorafenib), a modulator of post-translational modification (e.g., glycosylation inhibitor tunicamycin, ppGalNAc-T3), a modulator of protein degradation, and a modulator of protein transport (e.g., cyclosporin A, fendiline, parbendazole, paroxetine, parthenolide, quinacrine, sertraline, spiperone, thimerosal, astemizole, perhexiline, HUN-7293, CAM741, CK147, and cotransin, etc.) or a topoisomerase inhibitor.

In one aspect, the present disclosure relates to a method for selecting a patient for a cancer treatment, the method comprising: (a) obtaining a biological sample from a subject; (b) evaluating the biological sample for the expression of: (i) a gene selected from CD274, B2M, STAT1, STAT2, TRIM7, IRF1, TAP1, TAP2, CASP1, IRF, LTBR, PVR, GASTA3, LRG1, SPRY2, ARG1, TRIM8, TRIM2, MAPK8IP1, TRIM6, and KRT1; and/or (ii) a gene selected from RPL41, RPS15, RPS8, TRIM7 and LRG1; and (c) selecting the cancer therapy based on the evaluation of step (b).

In one aspect, the present disclosure relates to a method for selecting a patient for a cancer treatment, the method comprising: (a) obtaining a biological sample from a subject; (b) evaluating the biological sample for the expression of: (i) a gene selected from CD274, B2M, STAT1, STAT2, TRIM7, IRF1, TAP1, TAP2, CASP1, IRF, LTBR, PVR, GASTA3, LRG1, SPRY2, ARG1, TRIM8, TRIM2, MAPK8IP1, TRIM6, and KRT1; and/or (ii) a gene selected from RPL41, RPS15, RPS8, TRIM7 and LRG1; and (c) selecting the cancer therapy based on the evaluation of step (b), wherein cancer therapy is selected from: (i) an agent with an ability to inhibit function and/or activity of PD-1, PD-L1 and/or PD-L2; (ii) an antimetabolite chemotherapeutic (e.g., 5-fluorouracil, methotrexate, capecitabine, azacitidine, 6-diazo-5-oxo-L-norleucine (DON), azaserine and acivicin), a topoisomerase inhibitor (e.g., irinotecan, topotecan, etoposide, doxorubicin, etc.); and (iii) a protein translation inhibitor (e.g., silvestrol and omacetaxine) ribosome biogenesis inhibitors (e.g., diazaborine, lamotrigine and ribozinoindoles), inhibitors of rRNA and/or tRNA synthesis (e.g., quarfloxin (CX-3543) and CX-5461), an inhibitor of synthesis of amino acids (e.g., GLUD1 inhibitor R162, BCAT1 inhibitor gabapentin, glutaminase inhibitor bis-2-(5-phenylacetamido-1,2,4-thiadiazol-2-yl)ethyl sulfide (BPTES), PAGDH inhibitor NCT-503), an inhibitor of uptake of amino acids (e.g., SLC7A11 inhibitors sulfasalazine, erastin or sorafenib), a modulator of post-translational modification (e.g., glycosylation inhibitor tunicamycin, ppGalNAc-T3), a modulator of protein degradation, and a modulator of protein transport (e.g., cyclosporin A, fendiline, parbendazole, paroxetine, parthenolide, quinacrine, sertraline, spiperone, thimerosal, astemizole, perhexiline, HUN-7293, CAM741, CK147, and cotransin, etc.) or a topoisomerase inhibitor.

In one aspect, the present disclosure relates to a method of treating cancer, the method comprising: (a) obtaining a biological sample from a subject; (b) evaluating the biological sample for the expression of: (i) a gene selected from CD274, B2M, STAT1, STAT2, TRIM7, IRF1, TAP1, TAP2, CASP1, IRF, LTBR, PVR, GASTA3, LRG1, SPRY2, ARG1, TRIM8, TRIM2, MAPK8IP1, TRIM6, and KRT1; and/or (ii) a gene selected from RPL41, RPS15, RPS8, TRIM7 and LRG1; and (c) selecting the cancer therapy selected from: (i) an agent with an ability to inhibit function and/or activity of PD-1, PD-L1 and/or PD-L2; (ii) an antimetabolite chemotherapeutic (e.g., 5-fluorouracil, methotrexate, capecitabine, azacitidine, 6-diazo-5-oxo-L-norleucine (DON), azaserine and acivicin), a topoisomerase inhibitor (e.g., irinotecan, topotecan, etoposide, doxorubicin, etc.); and (iii) a protein translation inhibitor (e.g., silvestrol and omacetaxine) ribosome biogenesis inhibitors (e.g., diazaborine, lamotrigine and ribozinoindoles), inhibitors of rRNA and/or tRNA synthesis (e.g., quarfloxin (CX-3543) and CX-5461), an inhibitor of synthesis of amino acids (e.g., GLUD1 inhibitor R162, BCAT1 inhibitor gabapentin, glutaminase inhibitor bis-2-(5-phenylacetamido-1,2,4-thiadiazol-2-yl)ethyl sulfide (BPTES), PAGDH inhibitor NCT-503), an inhibitor of uptake of amino acids (e.g., SLC7A11 inhibitors sulfasalazine, erastin or sorafenib), a modulator of post-translational modification (e.g., glycosylation inhibitor tunicamycin, ppGalNAc-T3), a modulator of protein degradation, and a modulator of protein transport (e.g., cyclosporin A, fendiline, parbendazole, paroxetine, parthenolide, quinacrine, sertraline, spiperone, thimerosal, astemizole, perhexiline, HUN-7293, CAM741, CK147, and cotransin, etc.) or a topoisomerase inhibitor; and (d) administering the cancer therapy with an ability to inhibit function and/or activity of PD-1, PD-L1 and/or PD-L2.

In non-limiting embodiments, administering the cancer therapy with an ability to inhibit function and/or activity of PD-1 PD-L1 and/or PD-L2 is continued if the upregulation of the overexpression of a gene selected from CD274, B2M, STAT1, STAT2, TRIM7, IRF1, TAP1, TAP2, CASP1, IRF, LTBR, PVR, GASTA3, LRG1, SPRY2, ARG1, TRIM8, TRIM2, MAPK8IP1, TRIM6, and KRT1 is not observed; and/or downregulation a gene selected from RPL41, RPS15, RPS8, TRIM7 and LRG1 is not observed.

In non-limiting embodiments, administering the cancer therapy with an ability to inhibit function and/or activity of PD-1, PD-L1 and/or PD-L2 is continued if the upregulation of the overexpression of a gene selected from CD274, B2M, STAT1, STAT2, TRIM7, IRF1, TAP1, TAP2, CASP1, IRF, LTBR, PVR, GASTA3, LRG1, SPRY2, ARG1, TRIM8, TRIM2, MAPK8IP1, TRIM6, and KRT1 is observed; and/or downregulation a gene selected from RPL41, RPS15, RPS8, TRIM7 and LRG1 is observed, wherein the supplementation of administration of cancer therapy selected from: (i) an agent with an ability to inhibit function and/or activity of PD-1, PD-L1 and/or PD-L2; (ii) an antimetabolite chemotherapeutic (e.g., 5-fluorouracil, methotrexate, capecitabine, azacitidine, 6-diazo-5-oxo-L-norleucine (DON), azaserine and acivicin), a topoisomerase inhibitor (e.g., irinotecan, topotecan, etoposide, doxorubicin, etc.); and (iii) a protein translation inhibitor (e.g., silvestrol and omacetaxine) ribosome biogenesis inhibitors (e.g., diazaborine, lamotrigine and ribozinoindoles), inhibitors of rRNA and/or tRNA synthesis (e.g., quarfloxin (CX-3543) and CX-5461), an inhibitor of synthesis of amino acids (e.g., GLUD1 inhibitor R162, BCAT1 inhibitor gabapentin, glutaminase inhibitor bis-2-(5-phenylacetamido-1,2,4-thiadiazol-2-yl)ethyl sulfide (BPTES), PAGDH inhibitor NCT-503), an inhibitor of uptake of amino acids (e.g., SLC7A11 inhibitors sulfasalazine, erastin or sorafenib), a modulator of post-translational modification (e.g., glycosylation inhibitor tunicamycin, ppGalNAc-T3), a modulator of protein degradation, and a modulator of protein transport (e.g., cyclosporin A, fendiline, parbendazole, paroxetine, parthenolide, quinacrine, sertraline, spiperone, thimerosal, astemizole, perhexiline, HUN-7293, CAM741, CK147, and cotransin) is carried out.

In non-limiting embodiments, administering the cancer therapy with an ability to inhibit function and/or activity of PD-1, PD-L1 and/or PD-L2 is not continued if the upregulation of the overexpression of a gene selected from CD274, B2M, STAT1, STAT2, TRIM7, IRF1, TAP1, TAP2, CASP1, IRF, LTBR, PVR, GASTA3, LRG1, SPRY2, ARG1, TRIM8, TRIM2, MAPK8IP1, TRIM6, and KRT1 is observed; and/or downregulation a gene selected from RPL41, RPS15, RPS8, TRIM7 and LRG1 is observed, wherein the administration of cancer therapy selected from: (i) an agent with an ability to inhibit function and/or activity of PD-1, PD-L1 and/or PD-L2; (ii) an antimetabolite chemotherapeutic (e.g., 5-fluorouracil, methotrexate, capecitabine, azacitidine, 6-diazo-5-oxo-L-norleucine (DON), azaserine and acivicin), a topoisomerase inhibitor (e.g., irinotecan, topotecan, etoposide, doxorubicin, etc.); and (iii) a protein translation inhibitor (e.g., silvestrol and omacetaxine) ribosome biogenesis inhibitors (e.g., diazaborine, lamotrigine and ribozinoindoles), inhibitors of rRNA and/or tRNA synthesis (e.g., quarfloxin (CX-3543) and CX-5461), an inhibitor of synthesis of amino acids (e.g., GLUD1 inhibitor R162, BCAT1 inhibitor gabapentin, glutaminase inhibitor bis-2-(5-phenylacetamido-1,2,4-thiadiazol-2-yl)ethyl sulfide (BPTES), PAGDH inhibitor NCT-503), an inhibitor of uptake of amino acids (e.g., SLC7A11 inhibitors sulfasalazine, erastin or sorafenib), a modulator of post-translational modification (e.g., glycosylation inhibitor tunicamycin, ppGalNAc-T3), a modulator of protein degradation, and a modulator of protein transport (e.g., cyclosporin A, fendiline, parbendazole, paroxetine, parthenolide, quinacrine, sertraline, spiperone, thimerosal, astemizole, perhexiline, HUN-7293, CAM741, CK147, and cotransin) is carried out.

In embodiments, the cancer therapy with an ability to inhibit function and/or activity of PD-1, PD-L1 and/or PD-L2 is selected when the biological sample comprises at least one tumor cell, and a gene selected from CD274, B2M, STAT1, STAT2, TRIM7, IRF1, TAP1, TAP2, CASP1, IRF, LTBR, PVR, GASTA3, LRG1, SPRY2, ARG1, TRIM8, TRIM2, MAPK8IP1, TRIM6, and KRT1 is not upregulated in the at least one tumor cell compared to a healthy tissue, a prior biological sample obtained from the subject, or another biological sample from patient that is known to be sensitive to anti-PD-1 therapy; and/or a gene selected from RPL41, RPS15, RPS8, TRIM7 and LRG1 is not downregulated in the at least one tumor cell compared to a healthy tissue, a prior biological sample obtained from the subject, or another biological sample from patient that is known to be sensitive to anti-PD-1 therapy.

In embodiments, when the biological sample comprises at least one tumor cell, and a gene selected from CD274, B2M, STAT1, STAT2, TRIM7, IRF1, TAP1, TAP2, CASP1, IRF, LTBR, PVR, GASTA3, LRG1, SPRY2, ARG1, TRIM8, TRIM2, MAPK8IP1, TRIM6, and KRT1 is upregulated in the at least one tumor cell compared to a healthy tissue, a prior biological sample obtained from the subject, or another biological sample from patient that is known to be sensitive to anti-PD-1 therapy; and/or a gene selected from RPL41, RPS15, RPS8, TRIM7 and LRG1 is downregulated in the at least one tumor cell compared to a healthy tissue, a prior biological sample obtained from the subject, or another biological sample from patient that is known to be sensitive to anti-PD-1 therapy, the cancer therapy is selected from: an antimetabolite chemotherapeutic, a topoisomerase inhibitor, a protein translation inhibitor, a ribosome biogenesis inhibitor, an inhibitor of rRNA and/or tRNA synthesis, an inhibitor of synthesis of amino acids, an inhibitor of uptake of amino acids, a modulator of post-translational modification, a modulator of protein degradation, a modulator of protein transport, a topoisomerase inhibitor.

In embodiments, an upregulation of one or more genes listed in (b)(i) compared to a healthy tissue indicates a lack of response, resistance or recalcitrance to the cancer therapy with an ability to inhibit function and/or activity of PD-1, PD-L1 and/or PD-L2. In embodiments, an downregulation of one or more genes listed in (b)(ii) compared to a healthy tissue indicates a lack of response, resistance or recalcitrance to the cancer therapy with an ability to inhibit function and/or activity of PD-1, PD-L1 and/or PD-L2.

In embodiments, an upregulation of one or more genes listed in (b)(i) compared to another biological sample from patient that is known to be sensitive to anti-PD-1 therapy indicates a lack of response, resistance or recalcitrance to the cancer therapy with an ability to inhibit function and/or activity of PD-1, PD-L1 and/or PD-L2. In embodiments, an downregulation of one or more genes listed in (b)(ii) compared to another biological sample from patient that is known to be sensitive to anti-PD-1 therapy indicates a lack of response, resistance or recalcitrance to the cancer therapy with an ability to inhibit function and/or activity of PD-1, PD-L1 and/or PD-L2.

In embodiments, an upregulation of one or more genes listed in (b)(i) compared to a prior biological sample obtained from the subject indicates a development of a lack of response, resistance or recalcitrance to the cancer therapy with an ability to inhibit function and/or activity of PD-1, PD-L1 and/or PD-L2. In embodiments, an downregulation of one or more genes listed in (b)(ii) compared to a prior biological sample obtained from the subject indicates a development of a lack of response, resistance or recalcitrance to the cancer therapy with an ability to inhibit function and/or activity of PD-1, PD-L1 and/or PD-L2.

In embodiments, a lack of upregulation of one or more genes listed in (b)(i) compared to a healthy tissue indicates a response to the cancer therapy with an ability to inhibit function and/or activity of PD-1, PD-L1 and/or PD-L2. In embodiments, a lack of downregulation of one or more genes listed in (b)(ii) compared to a healthy tissue indicates a response to the cancer therapy with an ability to inhibit function and/or activity of PD-1, PD-L1 and/or PD-L2.

In embodiments, a lack of upregulation of one or more genes listed in (b)(i) compared to another biological sample from patient that is known to be sensitive to anti-PD-1 therapy indicates a response to the cancer therapy with an ability to inhibit function and/or activity of PD-1, PD-L1 and/or PD-L2. In embodiments, a lack of downregulation of one or more genes listed in (b)(ii) compared to another biological sample from patient that is known to be sensitive to anti-PD-1 therapy indicates a response to the cancer therapy with an ability to inhibit function and/or activity of PD-1, PD-L1 and/or PD-L2.

In embodiments, a lack of upregulation of one or more genes listed in (b)(i) compared to a prior biological sample obtained from the subject indicates a development of lack of a response to the cancer therapy with an ability to inhibit function and/or activity of PD-1, PD-L1 and/or PD-L2. In embodiments, a lack of downregulation of one or more genes listed in (b)(ii) compared to a prior biological sample obtained from the subject indicates a development of lack of a response to the cancer therapy with an ability to inhibit function and/or activity of PD-1, PD-L1 and/or PD-L2.

In embodiments, the biological sample is a fresh tissue sample, frozen tumor tissue specimen, cultured cells, circulating tumor cells, or a formalin-fixed paraffin-embedded tumor tissue specimen. In embodiments, the biological sample is a biopsy sample. In embodiments, the biopsy sample is selected from endoscopic biopsy, bone marrow biopsy, endoscopic biopsy (e.g., cystoscopy, bronchoscopy and colonoscopy), needle biopsy (e.g., fine-needle aspiration, core needle biopsy, vacuum-assisted biopsy, X-ray-assisted biopsy, computerized tomography (CT)-assisted biopsy, magnetic resonance imaging (MRI)-assisted biopsy and ultrasound-assisted biopsy), skin biopsy (e.g., shave biopsy, punch biopsy, and incisional biopsy) and surgical biopsy. In embodiments, the biological sample comprises a body fluid selected from blood, plasma, serum, lacrimal fluid, tears, bone marrow, blood, blood cells, ascites, tissue or fine needle biopsy sample, cell-containing body fluid, free floating nucleic acids, sputum, saliva, urine, cerebrospinal fluid, peritoneal fluid, pleural fluid, feces, lymph, gynecological fluid, skin swab, vaginal swab, oral swab, nasal swab, washing or lavage such as a ductal lavage or broncheoalveolar lavage, aspirate, scraping, bone marrow specimen, tissue biopsy specimen, surgical specimen, feces, other body fluids, secretions, and/or excretions, and/or cells therefrom. In embodiments, the biological sample is obtained by a technique selected from scrapes, swabs, and biopsy. In embodiments, the biological sample is obtained by use of brushes, (cotton) swabs, spatula, rinse/wash fluids, punch biopsy devices, puncture of cavities with needles or surgical instrumentation.

In embodiments, the biological sample comprises at least one tumor cell. In embodiments, the tumor is selected from Hodgkin's and non-Hodgkin's lymphoma, B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; or chronic myeloblastic leukemia, basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (e.g., that associated with brain tumors), Meigs' syndrome cancer; renal carcinoma; colorectal cancer; and adrenal cancer.

In embodiments, the evaluating is performed by DNA sequencing, RNA sequencing, immunohistochemical staining, western blotting, in cell western, immunofluorescent staining, ELISA, and fluorescent activating cell sorting (FACS) or a combination thereof. In embodiments, the evaluating is performed by contacting the sample with an agent that specifically binds to one or more proteins encoded by one or more genes listed in (b)(i) and/or (b)(ii). In embodiments, the agent that specifically binds to one or proteins comprises an antibody, antibody-like molecule or binding a fragment thereof. In embodiments, the evaluating is performed by contacting the sample with an agent that specifically binds to one or more of nucleic acids of one or more genes associated with a gene listed in (b)(i) and/or (b)(ii). In embodiments, the agent that specifically binds to one or more of the nucleic acids is a nucleic acid primer or probe.

In one aspect, the present disclosure relates to a method of determining a cancer treatment for a patient, the method comprising: (a) obtaining a biological sample from a subject; (b) evaluating the biological sample for the activation of a pathway selected from Mapk8ip1, Trim7, Elk1, Lrg1, Arg1, Rap1, and Ras; and (c) selecting the cancer therapy based on the evaluation of step (b), wherein cancer therapy is selected from: (i) an agent with an ability to inhibit function and/or activity of PD-1, PD-L1 and/or PD-L2; (ii) an antimetabolite chemotherapeutic (e.g., 5-fluorouracil, methotrexate, capecitabine, azacitidine, 6-diazo-5-oxo-L-norleucine (DON), azaserine and acivicin), a topoisomerase inhibitor (e.g., irinotecan, topotecan, etoposide, doxorubicin, etc.); and (iii) a protein translation inhibitor (e.g., silvestrol and omacetaxine) ribosome biogenesis inhibitors (e.g., diazaborine, lamotrigine and ribozinoindoles), inhibitors of rRNA and/or tRNA synthesis (e.g., quarfloxin (CX-3543) and CX-5461), an inhibitor of synthesis of amino acids (e.g., GLUD1 inhibitor R162, BCAT1 inhibitor gabapentin, glutaminase inhibitor bis-2-(5-phenylacetamido-1,2,4-thiadiazol-2-yl)ethyl sulfide (BPTES), PAGDH inhibitor NCT-503), an inhibitor of uptake of amino acids (e.g., SLC7A11 inhibitors sulfasalazine, erastin or sorafenib), a modulator of post-translational modification (e.g., glycosylation inhibitor tunicamycin, ppGalNAc-T3), a modulator of protein degradation, and a modulator of protein transport (e.g., cyclosporin A, fendiline, parbendazole, paroxetine, parthenolide, quinacrine, sertraline, spiperone, thimerosal, astemizole, perhexiline, HUN-7293, CAM741, CK147, and cotransin, etc.) or a topoisomerase inhibitor.

In one aspect, the present disclosure relates to a method for selecting a patient for a cancer treatment, the method comprising: (a) obtaining a biological sample from a subject; (b) evaluating the biological sample for the activation of a pathway selected from Mapk8ip1, Trim7, Elk1, Lrg1, Arg1, Rap1, and Ras; and (c) selecting the cancer therapy based on the evaluation of step (b), wherein cancer therapy is selected from: (i) an agent with an ability to inhibit function and/or activity of PD-1, PD-L1 and/or PD-L2; (ii) an antimetabolite chemotherapeutic (e.g., 5-fluorouracil, methotrexate, capecitabine, azacitidine, 6-diazo-5-oxo-L-norleucine (DON), azaserine and acivicin), a topoisomerase inhibitor (e.g., irinotecan, topotecan, etoposide, doxorubicin, etc.); and (iii) a protein translation inhibitor (e.g., silvestrol and omacetaxine) ribosome biogenesis inhibitors (e.g., diazaborine, lamotrigine and ribozinoindoles), inhibitors of rRNA and/or tRNA synthesis (e.g., quarfloxin (CX-3543) and CX-5461), an inhibitor of synthesis of amino acids (e.g., GLUD1 inhibitor R162, BCAT1 inhibitor gabapentin, glutaminase inhibitor bis-2-(5-phenylacetamido-1,2,4-thiadiazol-2-yl)ethyl sulfide (BPTES), PAGDH inhibitor NCT-503), an inhibitor of uptake of amino acids (e.g., SLC7A11 inhibitors sulfasalazine, erastin or sorafenib), a modulator of post-translational modification (e.g., glycosylation inhibitor tunicamycin, ppGalNAc-T3), a modulator of protein degradation, and a modulator of protein transport (e.g., cyclosporin A, fendiline, parbendazole, paroxetine, parthenolide, quinacrine, sertraline, spiperone, thimerosal, astemizole, perhexiline, HUN-7293, CAM741, CK147, and cotransin, etc.) or a topoisomerase inhibitor.

In one aspect, the present disclosure relates to a method of treating cancer, the method comprising: (a) obtaining a biological sample from a subject; (b) evaluating the biological sample for the activation of a pathway selected from Mapk8ip1, Trim7, Elk1, Lrg1, Arg1, Rap1, and Ras; and (c) selecting the cancer therapy selected from: (i) an agent with an ability to inhibit function and/or activity of PD-1, PD-L1 and/or PD-L2; (ii) an antimetabolite chemotherapeutic (e.g., 5-fluorouracil, methotrexate, capecitabine, azacitidine, 6-diazo-5-oxo-L-norleucine (DON), azaserine and acivicin), a topoisomerase inhibitor (e.g., irinotecan, topotecan, etoposide, doxorubicin, etc.); and (iii) a protein translation inhibitor (e.g., silvestrol and omacetaxine) ribosome biogenesis inhibitors (e.g., diazaborine, lamotrigine and ribozinoindoles), inhibitors of rRNA and/or tRNA synthesis (e.g., quarfloxin (CX-3543) and CX-5461), an inhibitor of synthesis of amino acids (e.g., GLUD1 inhibitor R162, BCAT1 inhibitor gabapentin, glutaminase inhibitor bis-2-(5-phenylacetamido-1,2,4-thiadiazol-2-yl)ethyl sulfide (BPTES), PAGDH inhibitor NCT-503), an inhibitor of uptake of amino acids (e.g., SLC7A11 inhibitors sulfasalazine, erastin or sorafenib), a modulator of post-translational modification (e.g., glycosylation inhibitor tunicamycin, ppGalNAc-T3), a modulator of protein degradation, and a modulator of protein transport (e.g., cyclosporin A, fendiline, parbendazole, paroxetine, parthenolide, quinacrine, sertraline, spiperone, thimerosal, astemizole, perhexiline, HUN-7293, CAM741, CK147, and cotransin, etc.) or a topoisomerase inhibitor; and (d) administering the cancer therapy with an ability to inhibit function and/or activity of PD-1, PD-L1 and/or PD-L2; and optionally administering the therapy selected in step (c)(ii) and (c)(iii).

In embodiments, the cancer therapy with an ability to inhibit function and/or activity of PD-1, PD-L1 and/or PD-L2 is selected when the biological sample comprises at least one tumor cell, and pathway selected from Mapk8ip1, Trim7, Elk1, Lrg1, Arg1, Rap1, and Ras is not upregulated in the at least one tumor cell compared to a healthy tissue, a prior biological sample obtained from the subject, or another biological sample from patient that is known to be sensitive to anti-PD-1 therapy.

In embodiments, when the biological sample comprises at least one tumor cell, and pathway selected from Mapk8ip1, Trim7, Elk1, Lrg1, Arg1, Rap1, and Ras is upregulated in the at least one tumor cell compared to a healthy tissue, a prior biological sample obtained from the subject, or another biological sample from patient that is known to be sensitive to anti-PD-1 therapy, the cancer therapy is selected from: an antimetabolite chemotherapeutic, a topoisomerase inhibitor, a protein translation inhibitor, a ribosome biogenesis inhibitor, an inhibitor of rRNA and/or tRNA synthesis, an inhibitor of synthesis of amino acids, an inhibitor of uptake of amino acids, a modulator of post-translational modification, a modulator of protein degradation, a modulator of protein transport, a topoisomerase inhibitor.

In embodiments, an upregulation of pathway selected from Mapk8ip1, Trim7, Elk1, Lrg1, Arg1, Rap1, and Ras compared to a healthy tissue indicates a lack of response, resistance or recalcitrance to the cancer therapy with an ability to inhibit function and/or activity of PD-1, PD-L1 and/or PD-L2. In embodiments, an upregulation of pathway selected from Mapk8ip1, Trim7, Elk1, Lrg1, Arg1, Rap1, and Ras compared to another biological sample from patient that is known to be sensitive to anti-PD-1 therapy indicates a lack of response, resistance or recalcitrance to the cancer therapy with an ability to inhibit function and/or activity of PD-1, PD-L1 and/or PD-L2. In embodiments, an upregulation of pathway selected from Mapk8ip1, Trim7, Elk1, Lrg1, Arg1, Rap1, and Ras compared to a prior biological sample obtained from the subject indicates a development of a lack of response, resistance or recalcitrance to the cancer therapy with an ability to inhibit function and/or activity of PD-1, PD-L1 and/or PD-L2.

In embodiments, a lack of upregulation of pathway selected from Mapk8ip1, Trim7, Elk1, Lrg1, Arg1, Rap1, and Ras compared to a healthy tissue indicates a response to the cancer therapy with an ability to inhibit function and/or activity of PD-1, PD-L1 and/or PD-L2. In embodiments, a lack of upregulation of pathway selected from Mapk8ip1, Trim7, Elk1, Lrg1, Arg1, Rap1, and Ras compared to another biological sample from patient that is known to be sensitive to anti-PD-1 therapy indicates a response to the cancer therapy with an ability to inhibit function and/or activity of PD-1, PD-L1 and/or PD-L2. In embodiments, in a lack of upregulation of pathway selected from Mapk8ip1, Trim7, Elk1, Lrg1, Arg1, Rap1, and Ras compared to a prior biological sample obtained from the subject indicates a development of lack of a response to the cancer therapy with an ability to inhibit function and/or activity of PD-1, PD-L1 and/or PD-L2.

In embodiments, the biological sample is a fresh tissue sample, frozen tumor tissue specimen, cultured cells, circulating tumor cells, or a formalin-fixed paraffin-embedded tumor tissue specimen. In embodiments, the biological sample is a biopsy sample. In embodiments, the biopsy sample is selected from endoscopic biopsy, bone marrow biopsy, endoscopic biopsy (e.g., cystoscopy, bronchoscopy and colonoscopy), needle biopsy (e.g., fine-needle aspiration, core needle biopsy, vacuum-assisted biopsy, X-ray-assisted biopsy, computerized tomography (CT)-assisted biopsy, magnetic resonance imaging (MRI)-assisted biopsy and ultrasound-assisted biopsy), skin biopsy (e.g., shave biopsy, punch biopsy, and incisional biopsy) and surgical biopsy. In embodiments, the biological sample comprises a body fluid selected from blood, plasma, serum, lacrimal fluid, tears, bone marrow, blood, blood cells, ascites, tissue or fine needle biopsy sample, cell-containing body fluid, free floating nucleic acids, sputum, saliva, urine, cerebrospinal fluid, peritoneal fluid, pleural fluid, feces, lymph, gynecological fluid, skin swab, vaginal swab, oral swab, nasal swab, washing or lavage such as a ductal lavage or broncheoalveolar lavage, aspirate, scraping, bone marrow specimen, tissue biopsy specimen, surgical specimen, feces, other body fluids, secretions, and/or excretions, and/or cells therefrom. In embodiments, the biological sample is obtained by a technique selected from scrapes, swabs, and biopsy. In embodiments, the biological sample is obtained by use of brushes, (cotton) swabs, spatula, rinse/wash fluids, punch biopsy devices, puncture of cavities with needles or surgical instrumentation.

In embodiments, the biological sample comprises at least one tumor cell. In embodiments, the tumor is selected from Hodgkin's and non-Hodgkin's lymphoma, B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; or chronic myeloblastic leukemia, basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (e.g., that associated with brain tumors), Meigs' syndrome cancer; renal carcinoma; colorectal cancer; and adrenal cancer.

In embodiments, the evaluating is performed by DNA sequencing, RNA sequencing, immunohistochemical staining, western blotting, in cell western, immunofluorescent staining, ELISA, and fluorescent activating cell sorting (FACS) or a combination thereof. In embodiments, the evaluating is performed by contacting the sample with an agent that specifically binds to one or more proteins encoded by a pathway selected from Mapk8ip1, Trim7, Elk1, Lrg1, Arg1, Rap1, and Ras. In embodiments, the agent that specifically binds to one or proteins comprises an antibody, antibody-like molecule or binding a fragment thereof. In embodiments, the evaluating is performed by contacting the sample with an agent that specifically binds to one or more of nucleic acids of one or more genes associated with a pathway selected from Mapk8ip1, Trim7, Elk1, Lrg1, Arg1, Rap1, and Ras. In embodiments, the agent that specifically binds to one or more of the nucleic acids is a nucleic acid primer or probe.

Trim7 is a member of a large family comprising approximately 80 distinct tripartite motif proteins (Tripartite motif-containing (TRIM)). The protein family comprises 80 TRIM protein members in humans. In embodiments, the Trim-family activation may be assayed by modulation of innate immune responses through the control of IFN responsive genes (i.e. IRF1/IRF3/IRF7, JAK/STAT, and NFκB. In embodiments, the Trim-family members have a C-terminal SPRY domain (e.g., in Trim6 and Trim7), In embodiments, Trim7 is assayed based on induction of proliferation, EMT, and acquisition of a chemo-resistant phenotype.

In embodiments, Trim7 pathway activation may be assayed using E3 ubiquitin ligase activity. In embodiments, Trim7 pathway activation may be assayed c-Jun/AP1 activation by Ras-Raf-MEK-ERK signaling. In embodiments, Trim7 pathway activation may be assayed by assaying protein ubiquitylation.

In embodiments, Trim7 pathway activation may be assayed using the ubiquitination and stabilization of AP1 co-activator RACO-1 and/or an increase in AP1 mediated gene expression. AP1-mediated gene expression signature is well known in the art.

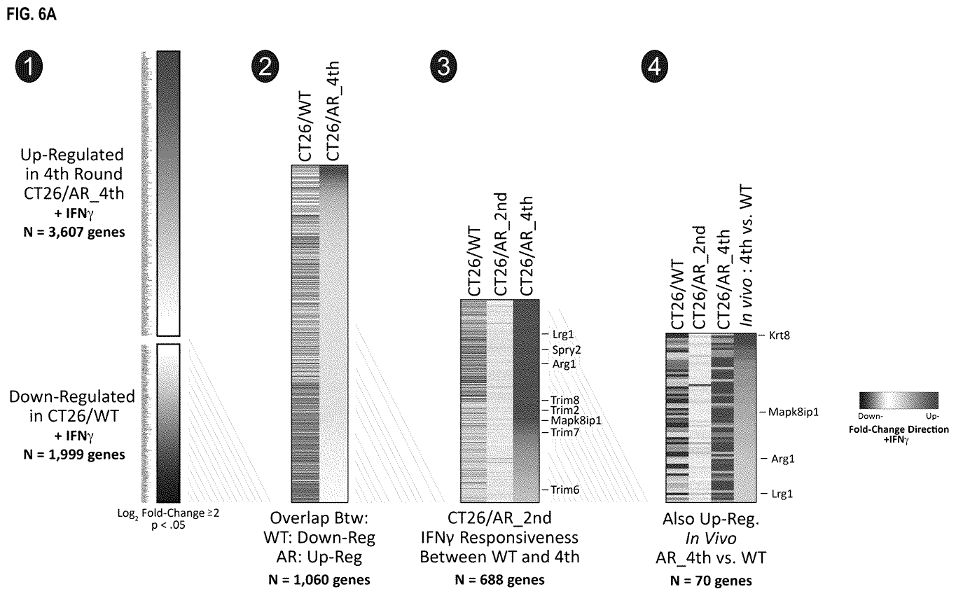

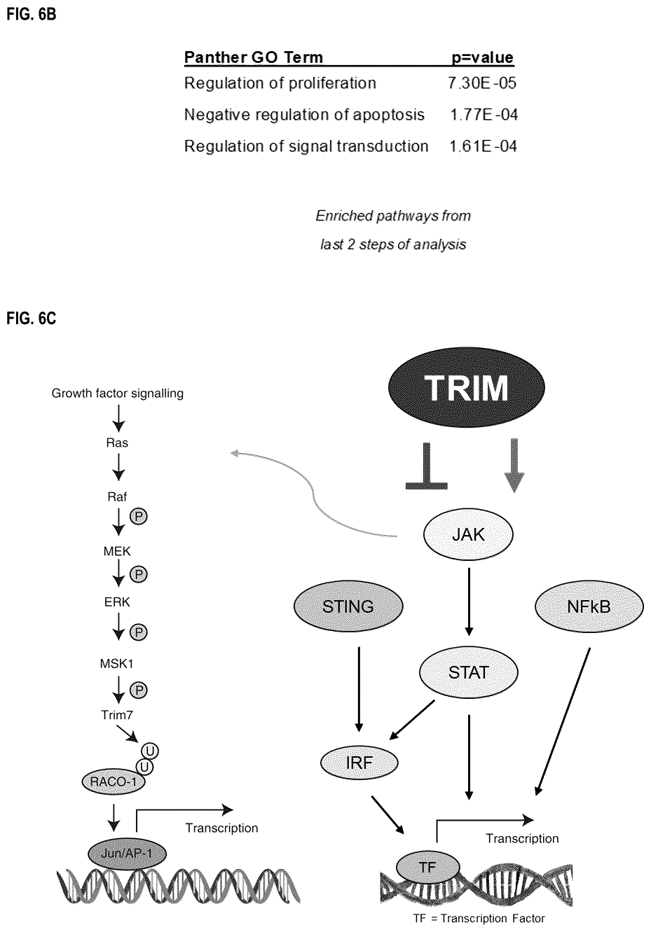

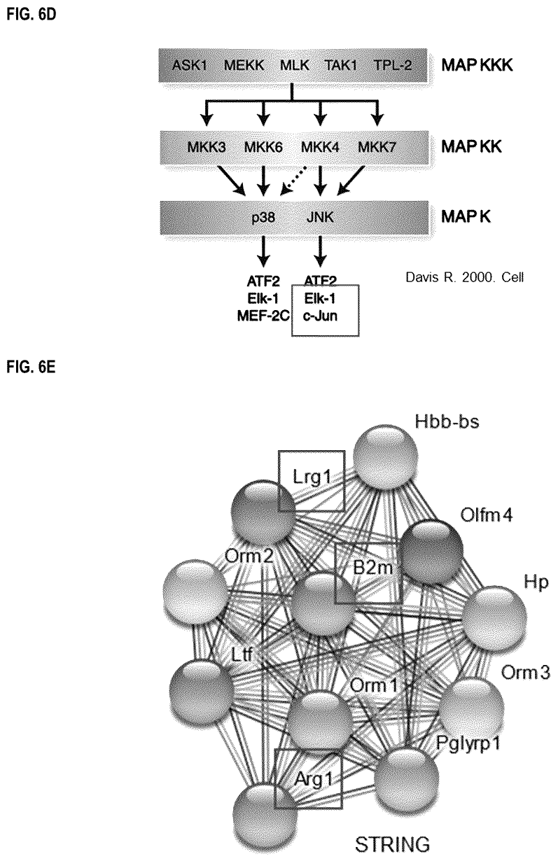

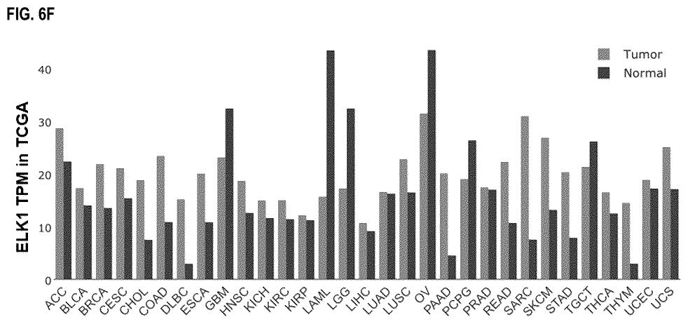

In embodiments, Trim7 pathway activation may be assayed based on K63-linked ubiquitylation of target proteins, including proteins involved with cell proliferation and innate immune responses. In embodiments, Trim7 pathway activation may be assayed based on Trim7 phosphorylation, K63-linked ubiquitylation and/or protein level of the AP-1 co-activator known as RACO-1. In embodiments, Trim7 pathway activation may be assayed based on the level or activity of STING (stimulator of interferon genes, MITA, ERIS, MPYS, TMEM173). In embodiments, Trim7 pathway activation may be assayed via K48-linked ubiquitylation of STING. In embodiments, Trim7 pathway activation may be assayed upregulation of IFNb, IP-10 and Rantes. In embodiments, Trim7 pathway activation may be assayed based on the activation of a protein shown in FIG. 6C.

Mitogen-activated protein kinase 8 interacting protein 1 (Mapk8ip1) is also known as c-Jun-amino-terminal kinase-interacting protein 1. In embodiments, Mapk8ip1 pathway activation may be assayed based on assay of functional multiprotein complex in different components of the JNK pathway, including RAC1 or RAC2, MAP3K11/MLK3 or MAP3K7/TAK1, MAP2K7/MKK7, MAPK8/JNK1 and/or MAPK9/JNK2. In embodiments, Mapk8ip1 pathway activation may be assayed based on levels of and/or sensitivity to programmed cell death by apoptosis.

In embodiments, ETS Like-1 protein (Elk1) pathway activation may be assayed based on the activation of Ras-Raf-MEK-ERK signaling, which is well known in the art. In embodiments, Elk1 pathway activation may be assayed based on Elk1 phosphorylation. In embodiments, Elk1 pathway activation may be assayed based on activation of Elk1 target genes, which are well-known in the art (see, e.g., Odrowaz and Sharrocks, ELK1 Uses Different DNA Binding Modes to Regulate Functionally Distinct Classes of Target Genes, PLOS Genetics 8(5):e1002694 (2012).

In embodiments, leucine-rich alpha-2-glycoprotein 1 (Lrg1) pathway activation may be assayed based on the induction of TGFβ and/or SMAD1/5/8 signaling.

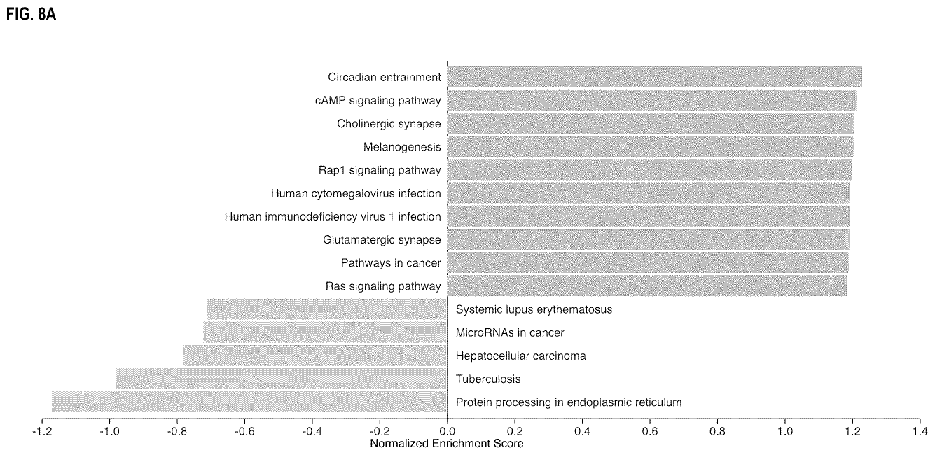

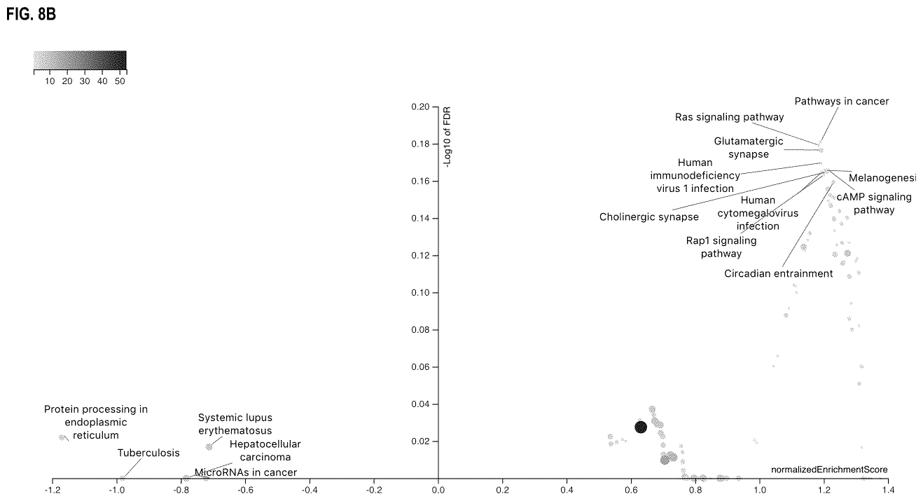

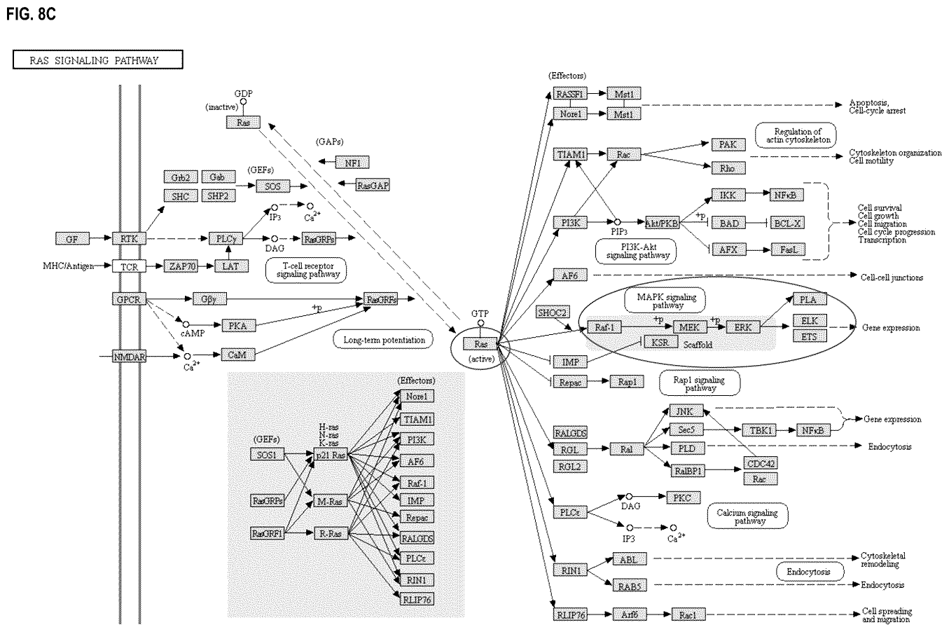

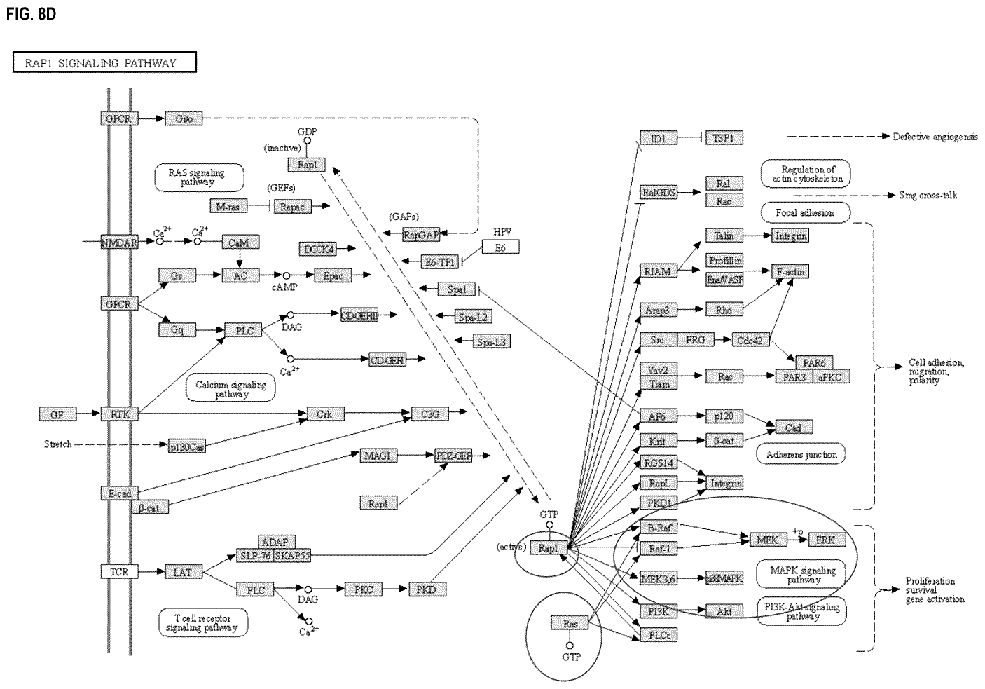

In embodiments, Ras pathway activation may be assayed based on the activation of a protein shown in FIG. 8C.

In embodiments, Rap1 pathway activation may be assayed based on the activation of a protein shown in FIG. 8D.

In embodiments, Arginase 1 (Arg1) pathway activation may be assayed based on the assay hydrolysis of arginine to ornithine and urea. In embodiments, Arginase 1 (Arg1) pathway activation may be assayed based on the suppression of tumor infiltrating lymphocytes (TILs). In embodiments, Arginase 1 (Arg1) pathway activation may be assayed based on the levels or synthesis of polyamines (via L-ornithine).

In one aspect, the present disclosure relates to a method of treating a cancer in a subject in need thereof, the method comprising administering a cancer therapy is selected from: an antimetabolite chemotherapeutic, a topoisomerase inhibitor, a protein translation inhibitor, a ribosome biogenesis inhibitor, an inhibitor of rRNA and/or tRNA synthesis, an inhibitor of synthesis of amino acids, an inhibitor of uptake of amino acids, a modulator of post-translational modification, a modulator of protein degradation, a modulator of protein transport, a topoisomerase inhibitor, wherein the subject has received or is receiving an anticancer treatment with an ability to inhibit function and/or activity of PD-1, PD-L1 and/or PD-L2, and wherein the subject has developed a lack of response, resistance or recalcitrance to the cancer therapy with an ability to inhibit function and/or activity of PD-1, PD-L1 and/or PD-L2.

In one aspect, the present disclosure relates to a method of treating a cancer in a subject in need thereof, the method comprising: (a) administering an anticancer treatment with an ability to inhibit function and/or activity of PD-1, PD-L1 and/or PD-L2; (b) evaluating anti-tumor response with the anticancer treatment with an ability to inhibit function and/or activity of PD-1, PD-L1 and/or PD-L2 by monitoring a tumor size reduction in the subject; (c) administering Trim7 modulator and/or a proteasome inhibitor if a lack of tumor size reduction is observed; (d) re-evaluating anti-tumor response with the anticancer treatment with an ability to inhibit function and/or activity of PD-1, PD-L1 and/or PD-L2 by monitoring the tumor reduction in the subject; and (e) withdrawing Trim7 modulator administration if a tumor size reduction is observed. In embodiments, the proteasome inhibitor is selected from bortezomib, carfilzomib, ixazomib, oprozomib, delanzomib and marizomib.

In one aspect, the present disclosure relates to a method of treating a cancer in a subject in need thereof, the method comprising: (a) administering an anticancer treatment with an ability to inhibit function and/or activity of PD-1, PD-L1 and/or PD-L2; (b) evaluating anti-tumor response with the anticancer treatment with an ability to inhibit function and/or activity of PD-1, PD-L1 and/or PD-L2 using the steps of: (i) obtaining a biological sample from the subject; (ii) evaluating the biological sample for the overexpression and/or activation of TRIM7; (c) administering Trim7 modulator and/or a proteasome inhibitor if an overexpression and/or activation of TRIM7 is observed; (d) re-evaluating anti-tumor response with the anticancer treatment with an ability to inhibit function and/or activity of PD-1, PD-L1 and/or PD-L2 using the steps of: (i) obtaining a biological sample from the subject; (ii) evaluating the biological sample for the overexpression and/or activation of TRIM7; and (e) withdrawing Trim7 modulator administration if an overexpression and/or activation of TRIM7 is not observed. In embodiments, the proteasome inhibitor is selected from bortezomib, carfilzomib, ixazomib, oprozomib, delanzomib and marizomib.

In one aspect, the present disclosure relates to a method of treating a cancer in a subject in need thereof, the method comprising: (a) administering an anticancer treatment with an ability to inhibit function and/or activity of PD-1, PD-L1 and/or PD-L2; (b) evaluating overexpression and/or activation of TRIM7 using the steps of: (i) obtaining a biological sample from the subject; and (ii) evaluating the biological sample for the overexpression and/or activation of TRIM7; (c) administering Trim7 modulator and/or a proteasome inhibitor if an overexpression and/or activation of TRIM7 is observed; (d) re-evaluating overexpression and/or activation of TRIM7 using the steps of: (i) obtaining a biological sample from the subject; (ii) evaluating the biological sample for the overexpression and/or activation of TRIM7; and (e) withdrawing Trim7 modulator administration if an overexpression and/or activation of TRIM7 is not observed. In embodiments, the proteasome inhibitor is selected from bortezomib, carfilzomib, ixazomib, oprozomib, delanzomib and marizomib.

In embodiments, the Trim7 modulator is a Trim 7 inhibitor. In embodiments, the Trim7 modulator is selected a small interference RNA (siRNA), a short hairpin RNA (shRNA), a microRNA (miRNA), an antisense RNA, a guide RNA (gRNA), a small molecule, an antibody, a peptide, and a peptidomimetic. In embodiments, the small interference RNA (siRNA), the short hairpin RNA (shRNA), the microRNA (miRNA), the antisense RNA, or the guide RNA (gRNA) inhibit the production of Trim7 protein. In embodiments, the peptidomimetic mimics a target of Trim7 and thereby inhibits the activity of Trim7.

In embodiments, wherein the Trim 7 inhibitor is a small molecule or peptide inhibitor that binds Trim7 protein at or near protein segments selected from MAAVGPRTGPGTGAEALALAAEL (SEQ ID NO: 1), AATRAPPFPLPCP (SEQ ID NO: 2), HGSQAAAARAAAARCG (SEQ ID NO: 3) and NVSLKTFVLKGMLKKFKEDLRGELEKEEKV (SEQ ID NO: 4).

In embodiments, the Trim7 modulator is an mitogen- and stress-activated kinase 1 (MSK1) inhibitor, wherein the MSK1 inhibitor modulates Trim7 via downstream effect of an inhibition of MSK1. In embodiments, the MSK1 inhibitor is selected from Ro 31-8220, SB-747651A, and H89. In embodiments, the MSK1 inhibitor is SB-747651A.

In embodiments, the proteasome inhibitor is selected from bortezomib, carfilzomib, ixazomib, oprozomib, delanzomib and marizomib.

In embodiments, the evaluating is performed by DNA sequencing, RNA sequencing, immunohistochemical staining, western blotting, in cell western, immunofluorescent staining, ELISA, and fluorescent activating cell sorting (FACS) or a combination thereof. In embodiments, the evaluating is performed by contacting the sample with an agent that specifically binds to one or more proteins encoded by the Trim7 pathway. In embodiments, the agent that specifically binds to one or proteins comprises an antibody, antibody-like molecule or binding a fragment thereof. In embodiments, the evaluating is performed by contacting the sample with an agent that specifically binds to one or more of nucleic acids of one or more genes associated with the Trim7 pathway. In embodiments, the agent that specifically binds to one or more of the nucleic acids is a nucleic acid primer or probe.

In embodiments, the evaluating is performed by assaying a E3 ubiquitin ligase activity. In embodiments, the evaluating is performed by assaying protein ubiquitylation and/or K48-linked ubiquitylation of stimulator of interferon genes (STING) and/or AP-1 co-activator RACO-1. In embodiments, the evaluating is performed by assaying c-Jun/AP1 activation via Ras-Raf-MEK-ERK signaling and/or an increase in AP1 mediated gene expression. In embodiments, the evaluating is performed by assaying ubiquitination and stabilization of AP1 co-activator RACO-1. In embodiments, the evaluating is performed by assaying K63-linked ubiquitylation of target proteins, including proteins involved with cell proliferation and innate immune responses. In embodiments, the evaluating is performed by assaying Trim7 phosphorylation, K63-linked ubiquitylation and/or protein level of the AP-1 co-activator RACO-1. In embodiments, the evaluating is performed by assaying the upregulation of IFNβ, IP-10 and/or Rantes.

In embodiments, the method further comprises administration of an anti-checkpoint agent. In embodiments, the anti-checkpoint agent an antibody selected from nivolumab (OPDIVO), pembrolizumab (KEYTRUDA), pidilizumab (CT-011, CURE TECH), MK-3475 (MERCK), BMS 936559, MPDL3280A (ROCHE), Cemiplimab (LIBTAYO), Atezolizumab (TECENTRIQ), Avelumab (BAVENCIO), and Durvalumab (imfinzi). In embodiments, the pharmaceutical composition comprising the cancer therapy and the anti-checkpoint agent are administered simultaneously or contemporaneously. In embodiments, the pharmaceutical composition comprising the cancer therapy is administered after the anti-checkpoint agent is administered. In embodiments, the pharmaceutical composition comprising the cancer therapy is administered before the anti-checkpoint agent is administered.

In embodiments, the anti-checkpoint agent an antibody selected from nivolumab (OPDIVO), pembrolizumab (KEYTRUDA), pidilizumab (CT-011, CURE TECH), MK-3475 (MERCK), BMS 936559, MPDL3280A (ROCHE), Cemiplimab (LIBTAYO), Atezolizumab (TECENTRIQ), Avelumab (BAVENCIO), and Durvalumab (imfinzi).

### Transgenic Non-Human Animal Models for Testing Cancer Therapy

In one aspect, the present disclosure relates to a transgenic non-human animal comprising one or more tumor cells, wherein the tumor cells have: (a) upregulation of one or more genes associated with a gene ontology (GO) pathway selected from: positive regulation of cell cycle process, regulation of G1/S transition, regulation of cell division, regulation of cell proliferation, positive regulation of IκB kinase/NFκB signaling, type I IFN signaling pathway, cellular response to IFNγ, positive regulation of IFNα production, positive regulation of defense response, positive regulation of IFNβ production, regulation of inflammatory response, regulation of innate immune response, negative regulation of antigen processing and presentation, and antigen processing, presentation of endogenous peptides via MHC class I; and/or (b) downregulation of one or more genes associated with a gene ontology (GO) pathway selected from: phospholipid efflux, negative regulation of fibrinolysis, chylomicron assembly, plasma membrane repair, SRP-dependent co-translational protein targeting to membrane, ribosomal small subunit assembly, phospholipid efflux, regulation of translation, mitochondrial respiratory chain complex I, mitochondrial translational elongation, DNA-dependent DNA replication, and ATP biosynthetic process. In embodiments, the transgenic non-human animal is transgenic.

In embodiments, the tumor cells are resistant to a cancer therapy with an ability to inhibit function and/or activity of PD-1, PD-L1 and/or PD-L2. In embodiments, the cancer therapy with an ability to inhibit function and/or activity of PD-1, PD-L1 and/or PD-L2 is an antibody. In embodiments, the antibody is selected from nivolumab (OPDIVO), pembrolizumab (KEYTRUDA), pidilizumab (CT-011, CURE TECH), MK-3475 (MERCK), BMS 936559, MPDL3280A (ROCHE), Cemiplimab (LIBTAYO), Atezolizumab (TECENTRIQ), Avelumab (BAVENCIO), and Durvalumab (imfinzi). In embodiments, the one or more tumor cells have: (a) upregulation of one or more genes associated with a gene ontology (GO) pathway selected from: positive regulation of cell cycle process, regulation of G1/S transition, regulation of cell division, regulation of cell proliferation, positive regulation of IκB kinase/NFκB signaling, type I IFN signaling pathway, cellular response to IFNγ, positive regulation of IFNα production, positive regulation of defense response, positive regulation of IFNβ production, regulation of inflammatory response, regulation of innate immune response, negative regulation of antigen processing and presentation, and antigen processing, presentation of endogenous peptides via MHC class I; and/or (b) downregulation of one or more genes associated with a gene ontology (GO) pathway selected from: phospholipid efflux, negative regulation of fibrinolysis, chylomicron assembly, plasma membrane repair, SRP-dependent co-translational protein targeting to membrane, ribosomal small subunit assembly, phospholipid efflux, regulation of translation, mitochondrial respiratory chain complex I, mitochondrial translational elongation, DNA-dependent DNA replication, and ATP biosynthetic process. In embodiments, the one or more tumor cells have an upregulation of one or more genes associated with cellular response to IFNγ. In embodiments, the one or more tumor cells have an upregulation of one or more genes associated with type I IFN signaling pathway.

The transgenic non-human animal can be any animal that is known to be useful for mimicking the human cancer. In embodiments, the transgenic non-human animal may be a pig, cow, dog, cat, horse, donkey, goat, sheep, llama, or non-human primate (e.g., chimp). In embodiments, the transgenic non-human animal is a mammal. In embodiments, the transgenic non-human animal may be a rodent, such as a rat, mouse, hamster, rabbit, or guinea pig. In a preferred embodiment, the transgenic non-human animal is a mouse. In a preferred embodiment, the transgenic non-human animal is a rat. In embodiments, the mouse belongs to BALB/c or C57BL/6 strain. Such mice can be purchased from different suppliers, e.g., from Charles River Laboratories. In embodiments, the transgenic non-human animal is a transgenic transgenic non-human animal (without limitation, e.g., a transgenic mouse). In embodiments, the transgenic transgenic non-human animal is a transgenic mouse. In embodiments, the one or more genetic change that causes spontaneous tumors and/or inducible tumors.

In embodiments, the formation of tumor in the transgenic transgenic non-human animal (e.g., mouse) is caused by a gene knock-out of one or more genes, optionally, the gene knock-out of one or more genes is inducible. In embodiments, the knock-out of one or more genes carried out using cre-loxP, CRISPR/Cas9, or the like, or a combination thereof. In embodiments, the knock-out of one or more genes is associated with an upregulation of one or more genes in the cells that have the knock-out of one or more genes. In embodiments, the knock-out of one or more genes is associated with a downregulation of one or more genes in the cells that have knock-out of one or more genes. In embodiments, the upregulation and the downregulation of one or more genes are independently optionally inducible. In embodiments, the upregulation and/or the downregulation is caused by placing one or more sequences (without limitation, e.g., an RNAi construct, a Cre recombinase construct and a gene knock-in construct) under the control of a promoter that controls expression of one or more genes.

In embodiments, the formation of tumor in the transgenic transgenic non-human animal (e.g., mouse) is caused by a knock-in of one or more tumor-causing-genes, optionally, the knock-in of one or more tumor-causing-genes is inducible. Such tumor-causing-genes are well-known in the art, and in embodiments include known oncogenes selected from c-Myc, HRAS^{G12V} or Kras^{G12D} and dominant negative p53 mutants. In embodiments, the knock-in of one or more tumor-causing-genes carried out using cre-loxP, CRISPR/Cas9, or the like, or a combination thereof. In embodiments, the knock-in of one or more tumor-causing-genes is associated with an upregulation of one or more genes in the cells that have the knock-in of one or more tumor-causing-genes. In embodiments, the knock-in of one or more tumor-causing-genes is associated with a downregulation of one or more genes in the cells that have knock-in of one or more tumor-causing-genes. In embodiments, the upregulation and the downregulation of one or more genes are independently optionally inducible. In embodiments, the upregulation and/or the downregulation is caused by placing one or more sequences (without limitation, e.g., an RNAi construct, a Cre recombinase construct and a further gene knock-in construct) under the control of a promoter that controls expression of one or more genes.

In embodiments, the formation of tumor in the transgenic transgenic non-human animal (e.g., mouse) is caused by a chromosomal translocation, optionally, the chromosomal translocation is inducible. In embodiments, the chromosomal translocation carried out using cre-loxP, CRISPR/Cas9, or the like, or a combination thereof. In embodiments, the chromosomal translocation is associated with an upregulation of one or more genes in the cells that have the chromosomal translocation. In embodiments, the chromosomal translocation is associated with a downregulation of one or more genes in the cells that have chromosomal translocation. In embodiments, the upregulation and the downregulation of one or more genes are independently optionally inducible. In embodiments, the upregulation and/or the downregulation is caused by placing one or more sequences (without limitation, e.g., an RNAi construct, a Cre recombinase construct and a gene knock-in construct) under the control of a promoter that controls expression of one or more genes.

In embodiments, the formation of tumor in the transgenic transgenic non-human animal (e.g., mouse) is caused by a chromosomal inversion, optionally, the chromosomal inversion is inducible. In embodiments, the chromosomal inversion carried out using cre-loxP, CRISPR/Cas9, or the like, or a combination thereof. In embodiments, the chromosomal inversion is associated with an upregulation of one or more genes in the cells that have the chromosomal inversion. In embodiments, the chromosomal inversion is associated with a downregulation of one or more genes in the cells that have chromosomal inversion. In embodiments, the upregulation and the downregulation of one or more genes are independently optionally inducible. In embodiments, the upregulation and/or the downregulation is caused by placing one or more sequences (without limitation, e.g., an RNAi construct, a Cre recombinase construct and a gene knock-in construct) under the control of a promoter that controls expression of one or more genes.

In any of the embodiments disclosed herein, the transgenic mouse harbors knock-in construct(s) that cause an upregulation of one or more genes associated with a gene ontology (GO) pathway selected from: positive regulation of cell cycle process, regulation of G1/S transition, regulation of cell division, regulation of cell proliferation, positive regulation of IκB kinase/NFκB signaling, type I IFN signaling pathway, cellular response to IFNγ, positive regulation of IFNα production, positive regulation of defense response, positive regulation of IFNβ production, regulation of inflammatory response, regulation of innate immune response, negative regulation of antigen processing and presentation, and antigen processing, presentation of endogenous peptides via MHC class I. In any of the embodiments disclosed herein, the transgenic mouse harbors knock-in construct(s) that causes a downregulation of one or more genes associated with a gene ontology (GO) pathway selected from: phospholipid efflux, negative regulation of fibrinolysis, chylomicron assembly, plasma membrane repair, SRP-dependent co-translational protein targeting to membrane, ribosomal small subunit assembly, phospholipid efflux, regulation of translation, mitochondrial respiratory chain complex I, mitochondrial translational elongation, DNA-dependent DNA replication, and ATP biosynthetic process.

In any of the embodiments disclosed herein, the transgenic mouse has a promoter that controls expression of one or more genes causing the upregulation of one or more genes and/or the downregulation of one or more genes via e.g., placing an RNAi construct, a Cre recombinase and/or a gene knock-in construct under the control of the promoter. In embodiments, the promoter is a strong promoter. In embodiments, the promoter is an inducible promoter, which allows for the controlled and independent upregulation and/or the downregulation of one or more genes. In embodiments, the promoter is selected from a doxycycline-inducible promoter, a tamoxifen-inducible promoter and a cumate-inducible promoter. In embodiments, the promoter is the promoter may be a tissue-specific promoter. In embodiments, the tissue-specific promoter is selected from mouse mammary tumor virus (MMTV) or whey acidic protein (WAP) promoter for expression in mammary gland, mouse pro-opiomelanocortin-alpha (POMC) promoter for central nervous system (hippocampus)-specific expression, human caudal type homeo-box 2 (CDX2) promoter for colonic epithelium-specific expression, mouse SRY-box containing gene 2 (Sox2) promoter for epiblast cell-specific expression, gamma-glutamyltransferase 1 (Ggt1) promoter for kidney epithelium-specific expression, albumin promoter/enhancer (Alb) for liver (hepatocytes)-specific expression, human surfactant pulmonary-associated protein C (SFTPC) promoter for lung (endoderm)-specific expression, pancreatic and duodenal homeobox1 (pdx1) promoter for pancreatic epithelium-specific expression, rat insulin II promoter for pancreatic β-cell-specific expression and leucine-rich repeat-containing G-protein coupled receptor 5 (Lgr5), fatty acid binding protein (Fabp) or mouse villin 1 promoter for small and large intestine (crypts)-specific expression.

Methods for the generation of a transgenic mouse models in cancer research are widely known in the art (Walrath et al., Genetically Engineered Mouse Models in Cancer Research, Adv Cancer Res. 106: 113-164 (2010)). These methods usually include the construction of a targeting vector that harbors the modified sequence that will be introduced into murine embryonic stem cells. The genetically altered embryonic stem cells are then inserted into a mouse. The blastocyst is implanted into the uterus of female mice which ultimately results in chimeric offspring. Heterozygous mice are then interbred to provide mice that are homozygous for the knocked-out gene and/or knock-in. Alternative methods for gene inactivation comprise the CRISPR/Cas9 system (Beil-Wagner et al. (2016) Sci Rep., 6: 21377). Methods for incorporating a modified promoter sequence are known in the art and include, as described above, the use of a targeting vector that will be introduced into embryonic stem cells of the animal. The knocked-out embryonic stem cells are then inserted into a blastocyst of the animal to generate offspring, which is then interbred to provide homozygous mice. Alternatively, methods for promoter modification in the genome may comprise the CRISPR/Cas9 system. Apart from modifications of the genome of the animal, it will also be possible to achieve production of a protein in a transgenic non-human animal by relying on viral or non-viral expression vectors.

Some methods include injection of a vector harboring a nucleic acid construct directing the desired changes in mice. In embodiments, the vectors are DNA vectors. In embodiments, the vectors are viral vectors. The vector then reaches a desired tissue and induces a desired genetic change in the mice.

Expression vectors that allow for the expression of a transgene in a transgenic non-human animal, such as a mouse, are known. Useful mammalian expression vectors have been described in the prior art and such expression systems are purchasable by different manufacturers. Useful expression systems include plasmid- or viral vector based systems, e.g., pLIVE (Minis Bio), LENTI-Smart (InvivoGen), GenScript Expression vectors, pAdVAntage (Promega), ViraPower Lentiviral, Adenoviral Expression Systems (Invitrogen) and adeno-associated viral expression systems (Cell Biolabs).

A suitable mammalian expression vector will normally comprise a promoter, which is functionally linked to the nucleic acid encoding one or more genes that are upregulated and/or the downregulated. The promoter sequence must be compact and ensure a strong expression. Suitable promoters include, but are not limited to, the cytomegalovirus promoter (CMV), the Spleen Focus Forming Virus (SFFV) U3 promoter and the adenoviral major late promoter (Ad MLP) and the promoters discussed above. As an optional component, the mammalian expression vector may include a suitable enhancer element for increasing the expression level. Examples include the SV40 early gene enhancer (Dijkema et al (1985) EMBO J. 4: 761) and the enhancer of the long terminal repeat (LTR) of Rous Sarcoma Virus (Gorman et al. (1982b) Proc. Natl. Acad. Sci. 79: 6777). The expression vector also optionally comprises transcription termination sequences and polyadenylation sequences for improved expression of the gene encoding the one or more genes that are upregulated and/or the downregulated. Suitable transcription terminator and polyadenylation signals can, for example, be derived from SV40 (Sambrook et al (1989), Molecular Cloning: A Laboratory Manual). Any other element which is known in the art to support efficient expression may be added to the expression vector, such as the Woodchuck hepatitis post-transcriptional regulatory element (wPRE). In embodiments, the vector may be a vector that can be administered to the animal by injection. In embodiments, the mammalian expression vector is a pLIVE expression vector (Minis Bio).

In embodiments, the vector is an expression vector, optionally which is a viral expression vector. Viral vectors typically comprise a viral genome in which a portion of the native sequence has been deleted in order to introduce a heterogeneous polynucleotide without destroying the infectivity of the virus. Due to the specific interaction between virus components and host cell receptors, viral vectors are highly suitable for efficient transfer of genes into target cells. Suitable viral vectors for facilitating gene transfer into a mammalian cell or organism are well known in the art and can be derived from different types of viruses, for example, from a retrovirus, adenovirus, adeno-associated virus (AAV), orthomyxovirus, paramyxovirus, papovavirus, picornavirus, lentivirus, herpes simplex virus, vaccinia virus, pox virus or alphavirus. For an overview of the different viral vector systems, see Nienhuis et al., Hematology, Vol. 16: Viruses and Bone Marrow, N. S. Young (ed.), 353-414 (1993).

In embodiments, the vector is an adeno-associated viral (AVV) vector, such as an AAV vector selected from the group consisting of serotype 1, 2, 3, 4, 5, 6, 7, 8, 9, and 10 or chimeric AAV derived thereof, which will be even better suitable for high efficiency transduction in the tissue of interest (Wu et al, 2006, Mol Therapy 14:316-27; Bowles et al, 2012, Mol Therapy 20:443-455). Upon transfection, AAV elicits only a minor immune reaction (if any) in the host. Moreover, in contrast to other vector systems AAV vectors are also able to efficiently pass from the blood into terminally differentiated cells. Therefore, in embodiments, AAV is highly suited for gene transfer approaches. In embodiments, for transduction in mice, AAV serotype 6 and AAV serotype 9 are used.

Recombinant viral vectors can be generated according to standard techniques. For example, recombinant adenoviral or adeno-associated viral vectors can be propagated in human 293 cells (which provide E1A and E1B functions in trans) to titers in the range of 107-1013 viral particles/mL. Prior to their in vivo application viral vectors may be desalted by gel filtration methods, such as sepharose columns, and purified by subsequent filtering. Purification reduces potential deleterious effects in the subject to which the vectors are administered. The administered virus is substantially free of wild-type and replication-competent virus. The purity of the virus can be proven by suitable methods, such as sodium dodecyl sulphate-polyacrylamide gel electrophoresis (SDS-PAGE) followed by silver staining. This is applicable for both AAV and adenoviral vectors.

Transduction of the vectors into the transgenic non-human animal can be achieved by systemic application, e.g., by intravenous (including hydrodynamic tail vein injection), intraarterial or intraperitoneal delivery of a vector. In a preferred embodiment, the vectors are administered systemically.

### Methods of Making Transgenic Non-Human Animal Models for Testing Cancer Therapy

In one aspect, the present disclosure relates to a method of making a transgenic non-human animal comprising one or more cancer cells that are nonresponsive, resistant, or recalcitrance to a cancer therapy, wherein the cancer therapy has an ability to inhibit function and/or activity of PD-1, PD-L1 and/or PD-L2, the method comprising: (a) injecting one or more parental cancer cells that are responsive to the cancer therapy in a non-human animal; (b) administering the cancer therapy to the non-human animal; (c) isolating cancer cells that survived the cancer therapy; (d) injecting cancer cells that survived the cancer therapy in a different non-human animal of the same species; and (e) repeating steps (b) to (d) two to ten more times.

In embodiments, steps (b) to (d) are repeated at least once more. In embodiments, steps (b) to (d) are repeated at least twice more. In embodiments, steps (b) to (d) are repeated at least three times more. In embodiments, steps (b) to (d) are repeated less than five times. In embodiments, the transgenic non-human animal is a rodent. In embodiments, the rodent is a mouse.

In embodiments, the mouse belongs to BALB/c or C57BL/6 strain. In embodiments, the cancer therapy that has the ability inhibit function and/or activity of PD-1, PD-L1 and/or PD-L2 is an antibody. In embodiments, the antibody is selected from nivolumab (OPDIVO), pembrolizumab (KEYTRUDA), pidilizumab (CT-011, CURE TECH), MK-3475 (MERCK), BMS 936559, MPDL3280A (ROCHE), Cemiplimab (LIBTAYO), Atezolizumab (TECENTRIQ), Avelumab (BAVENCIO), and Durvalumab (imfinzi).

In embodiments, the cancer therapy is capable of inhibiting the growth of tumors when administered to a transgenic non-human animal transgenic non-human animal harboring a parental cancer cell tumor compared to an untreated transgenic non-human animal harboring a parental cancer cell tumors. In embodiments, the tumor cells that survived the cancer therapy have: (a) upregulation of one or more genes associated with a gene ontology (GO) pathway selected from: positive regulation of cell cycle process, regulation of G1/S transition, regulation of cell division, regulation of cell proliferation, positive regulation of IκB kinase/NFκB signaling, type I IFN signaling pathway, cellular response to IFNγ, positive regulation of IFNα production, positive regulation of defense response, positive regulation of IFNβ production, regulation of inflammatory response, regulation of innate immune response, negative regulation of antigen processing and presentation, and antigen processing, presentation of endogenous peptides via MHC class I; and/or (b) downregulation of one or more genes associated with a gene ontology (GO) pathway selected from: phospholipid efflux, negative regulation of fibrinolysis, chylomicron assembly, plasma membrane repair, SRP-dependent co-translational protein targeting to membrane, ribosomal small subunit assembly, phospholipid efflux, regulation of translation, mitochondrial respiratory chain complex I, mitochondrial translational elongation, DNA-dependent DNA replication, and ATP biosynthetic process.

In embodiments, the one or more tumor cells have an upregulation of one or more genes associated with cellular response to IFNγ. In embodiments, the one or more tumor cells have an upregulation of one or more genes associated with type I IFN signaling pathway.

In one aspect, the present disclosure relates to a transgenic animal made according to the method of any of the embodiments disclosed herein.

### Methods of Testing Cancer Therapy and Making a Pharmaceutical Composition for Treating Cancer

In one aspect, the present disclosure relates to a method for testing an anti-cancer drug candidate, the method comprising: (a) providing a transgenic non-human animal of any of the embodiments disclosed herein, or a transgenic non-human animal made according to any of the embodiments disclosed herein; (b) administering the anti-cancer drug candidate to the transgenic non-human animal, and (c) evaluating whether the anti-cancer drug candidate is effective in slowing or inhibiting cancer growth in the transgenic non-human animal. In one aspect, the present disclosure relates to the anticancer drug candidate is selected from a chemotherapeutic agent, a cytotoxic agent, and a checkpoint inhibitor.

In one aspect, the present disclosure relates to a method for making a pharmaceutical composition for treating cancer, the method comprising: (a) providing a transgenic non-human animal of any of the embodiments disclosed herein, or a transgenic non-human animal made according to any of the embodiments disclosed herein; (b) administering the anti-cancer drug candidate to the transgenic non-human animal, and (c) selecting an anti-cancer drug that is effective in slowing or inhibiting cancer growth in the transgenic non-human animal; and (d) formulating the anti-cancer drug or candidate for administration to a human patient. In embodiments the anti-cancer drug candidate is selected from a chemotherapeutic agent, a cytotoxic agent, and a checkpoint inhibitor.

### Formulations

The cancer therapy (and/or additional agents) described herein can possess a sufficiently basic functional group, which can react with an inorganic or organic acid, or a carboxyl group, which can react with an inorganic or organic base, to form a pharmaceutically acceptable salt. A pharmaceutically acceptable acid addition salt is formed from a pharmaceutically acceptable acid, as is well known in the art. Such salts include the pharmaceutically acceptable salts listed in, for example, Journal of Pharmaceutical Science, 66, 2-19 (1977) and The Handbook of Pharmaceutical Salts; Properties, Selection, and Use. P. H. Stahl and C. G. Wermuth (eds.), Verlag, Zurich (Switzerland) 2002, which are hereby incorporated by reference in their entirety.

In embodiments, the compositions described herein are in the form of a pharmaceutically acceptable salt.

Further, any cancer therapy (and/or additional agents) described herein can be administered to a subject as a component of a composition that comprises a pharmaceutically acceptable carrier or vehicle. Such compositions can optionally comprise a suitable amount of a pharmaceutically acceptable excipient so as to provide the form for proper administration. Pharmaceutical excipients can be liquids, such as water and oils, including those of petroleum, animal, vegetable, or synthetic origin, such as peanut oil, soybean oil, mineral oil, sesame oil and the like. The pharmaceutical excipients can be, for example, saline, gum acacia, gelatin, starch paste, talc, keratin, colloidal silica, urea and the like. In addition, auxiliary, stabilizing, thickening, lubricating, and coloring agents can be used. In one embodiment, the pharmaceutically acceptable excipients are sterile when administered to a subject. Water is a useful excipient when any agent described herein is administered intravenously. Saline solutions and aqueous dextrose and glycerol solutions can also be employed as liquid excipients, specifically for injectable solutions. Suitable pharmaceutical excipients also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Any agent described herein, if desired, can also comprise minor amounts of wetting or emulsifying agents, or pH buffering agents.

In embodiments, the compositions described herein are resuspended in a saline buffer (including, without limitation TBS, PBS, and the like).

### Administration, Dosing, and Treatment Regimens

The present disclosure includes the described cancer therapy (and/or additional agents) in various formulations. Any cancer therapy (and/or additional agents) described herein can take the form of solutions, suspensions, emulsion, drops, tablets, pills, pellets, capsules, capsules containing liquids, powders, sustained-release formulations, suppositories, emulsions, aerosols, sprays, suspensions, or any other form suitable for use. DNA or RNA constructs encoding the protein sequences may also be used. In one embodiment, the composition is in the form of a capsule (see, e.g., U.S. Pat. No. 5,698,155). Other examples of suitable pharmaceutical excipients are described in Remington's Pharmaceutical Sciences 1447-1676 (Alfonso R. Gennaro eds., 19th ed. 1995), incorporated herein by reference.

Where necessary, the formulations comprising the cancer therapy (and/or additional agents) can also include a solubilizing agent. Also, the agents can be delivered with a suitable vehicle or delivery device as known in the art. Combination therapies outlined herein can be co-delivered in a single delivery vehicle or delivery device. Compositions for administration can optionally include a local anesthetic such as, for example, lignocaine to lessen pain at the site of the injection.

The formulations comprising the cancer therapy (and/or additional agents) of the present disclosure may conveniently be presented in unit dosage forms and may be prepared by any of the methods well known in the art of pharmacy. Such methods generally include the step of bringing therapeutic agents into association with a carrier, which constitutes one or more accessory ingredients. Typically, the formulations are prepared by uniformly and intimately bringing therapeutic agent into association with a liquid carrier, a finely divided solid carrier, or both, and then, if necessary, shaping the product into dosage forms of the desired formulation (e.g., wet or dry granulation, powder blends, etc., followed by tableting using conventional methods known in the art)

In one embodiment, any cancer therapy (and/or additional agents) described herein is formulated in accordance with routine procedures as a composition adapted for a mode of administration described herein.

Routes of administration include, for example: intradermal, intramuscular, intraperitoneal, intravenous, subcutaneous, intranasal, epidural, oral, sublingual, intranasal, intracerebral, intravaginal, transdermal, rectally, by inhalation, or topically, particularly to the ears, nose, eyes, or skin. In embodiments, the administering is effected orally or by parenteral injection. In most instances, administration results in the release of any agent described herein into the bloodstream.

Any cancer therapy (and/or additional agents) described herein can be administered orally. Such cancer therapy (and/or additional agents) can also be administered by any other convenient route, for example, by intravenous infusion or bolus injection, by absorption through epithelial or mucocutaneous linings (e.g., oral mucosa, rectal and intestinal mucosa, etc.) and can be administered together with another biologically active agent. Administration can be systemic or local. Various delivery systems are known, e.g., encapsulation in liposomes, microparticles, microcapsules, capsules, etc., and can be used to administer.

In specific embodiments, it may be desirable to administer locally to the area in need of treatment. In one embodiment, for instance in the treatment of cancer, the cancer therapy (and/or additional agents) are administered in the tumor microenvironment (e.g., cells, molecules, extracellular matrix and/or blood vessels that surround and/or feed a tumor cell, inclusive of, for example, tumor vasculature; tumor-infiltrating lymphocytes; fibroblast reticular cells; endothelial progenitor cells (EPC); cancer-associated fibroblasts; pericytes; other stromal cells; components of the extracellular matrix (ECM); dendritic cells; antigen presenting cells; T-cells; regulatory T cells; macrophages; neutrophils; and other immune cells located proximal to a tumor) or lymph node and/or targeted to the tumor microenvironment or lymph node. In embodiments, for instance in the treatment of cancer, the cancer therapy (and/or additional agents) are administered intratumorally.

Dosage forms suitable for parenteral administration (e.g., intravenous, intramuscular, intraperitoneal, subcutaneous and intra-articular injection and infusion) include, for example, solutions, suspensions, dispersions, emulsions, and the like. They may also be manufactured in the form of sterile solid compositions (e.g., lyophilized composition), which can be dissolved or suspended in sterile injectable medium immediately before use. They may contain, for example, suspending or dispersing agents known in the art.

The dosage of any cancer therapy (and/or additional agents) described herein as well as the dosing schedule can depend on various parameters, including, but not limited to, the disease being treated, the subject's general health, and the administering physician's discretion. Any cancer therapy described herein, can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concurrently with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of an additional agent, to a subject in need thereof. In embodiments any cancer therapy and additional agent described herein are administered 1 minute apart, 10 minutes apart, 30 minutes apart, less than 1 hour apart, 1 hour apart, 1 hour to 2 hours apart, 2 hours to 3 hours apart, 3 hours to 4 hours apart, 4 hours to 5 hours apart, 5 hours to 6 hours apart, 6 hours to 7 hours apart, 7 hours to 8 hours apart, 8 hours to 9 hours apart, 9 hours to 10 hours apart, 10 hours to 11 hours apart, 11 hours to 12 hours apart, 1 day apart, 2 days apart, 3 days apart, 4 days apart, 5 days apart, 6 days apart, 1 week apart, 2 weeks apart, 3 weeks apart, or 4 weeks apart.

The dosage of any cancer therapy (and/or additional agents) described herein can depend on several factors including the severity of the condition, whether the condition is to be treated or prevented, and the age, weight, and health of the subject to be treated. Additionally, pharmacogenomic (the effect of genotype on the pharmacokinetic, pharmacodynamic or efficacy profile of a therapeutic) information about a particular subject may affect dosage used. Furthermore, the exact individual dosages can be adjusted somewhat depending on a variety of factors, including the specific combination of the agents being administered, the time of administration, the route of administration, the nature of the formulation, the rate of excretion, the particular disease being treated, the severity of the disorder, and the anatomical location of the disorder. Some variations in the dosage can be expected.

For administration of any cancer therapy (and/or additional agents) described herein by parenteral injection, the dosage may be about 0.1 mg to about 250 mg per day, about 1 mg to about 20 mg per day, or about 3 mg to about 5 mg per day. Generally, when orally or parenterally administered, the dosage of any agent described herein may be about 0.1 mg to about 1500 mg per day, or about 0.5 mg to about 10 mg per day, or about 0.5 mg to about 5 mg per day, or about 200 to about 1,200 mg per day (e.g., about 200 mg, about 300 mg, about 400 mg, about 500 mg, about 600 mg, about 700 mg, about 800 mg, about 900 mg, about 1,000 mg, about 1,100 mg, about 1,200 mg per day).

In embodiments, administration of the cancer therapy (and/or additional agents) described herein is by parenteral injection at a dosage of about 0.1 mg to about 1500 mg per treatment, or about 0.5 mg to about mg per treatment, or about 0.5 mg to about 5 mg per treatment, or about 200 to about 1,200 mg per treatment (e.g., about 200 mg, about 300 mg, about 400 mg, about 500 mg, about 600 mg, about 700 mg, about 800 mg, about 900 mg, about 1,000 mg, about 1,100 mg, about 1,200 mg per treatment).

In embodiments, a suitable dosage of the cancer therapy (and/or additional agents) is in a range of about 0.01 mg/kg to about 100 mg/kg of body weight, or about 0.01 mg/kg to about 10 mg/kg of body weight of the subject, for example, about 0.01 mg/kg, about 0.02 mg/kg, about 0.03 mg/kg, about 0.04 mg/kg, about 0.05 mg/kg, about 0.06 mg/kg, about 0.07 mg/kg, about 0.08 mg/kg, about 0.09 mg/kg, about 0.1 mg/kg, about mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1 mg/kg, about 1.1 mg/kg, about 1.2 mg/kg, about 1.3 mg/kg, about 1.4 mg/kg, about 1.5 mg/kg, about 1.6 mg/kg, about 1.7 mg/kg, about 1.8 mg/kg, 1.9 mg/kg, about 2 mg/kg, about 3 mg/kg, about 4 mg/kg, about 5 mg/kg, about 6 mg/kg, about 7 mg/kg, about 8 mg/kg, about 9 mg/kg, about mg/kg, about 20 mg/kg, about 30 mg/kg, about 40 mg/kg, about 50 mg/kg, about 60 mg/kg, about 70 mg/kg, about 80 mg/kg, about 90 mg/kg, about 100 mg/kg body weight, inclusive of all values and ranges therebetween.

In another embodiment, delivery can be in a vesicle, in particular a liposome (see Langer, 1990, Science 249:1527-1533; Treat et al., in Liposomes in therapy of Infectious Disease and Cancer, Lopez-Berestein and Fidler (eds.), Liss, New York, pp. 353-365 (1989).

Any cancer therapy (and/or additional agents) described herein can be administered by controlled-release or sustained-release means or by delivery devices that are well known to those of ordinary skill in the art. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; and 5,733,556, each of which is incorporated herein by reference in its entirety. Such dosage forms can be useful for providing controlled- or sustained-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Controlled- or sustained-release of an active ingredient can be stimulated by various conditions, including but not limited to, changes in pH, changes in temperature, stimulation by an appropriate wavelength of light, concentration or availability of enzymes, concentration or availability of water, or other physiological conditions or compounds.

In another embodiment, polymeric materials can be used (see Medical Applications of Controlled Release, Langer and Wise (eds.), CRC Pres., Boca Raton, Florida (1974); Controlled Drug Bioavailability, Drug Product Design and Performance, Smolen and Ball (eds.), Wiley, New York (1984); Ranger and Peppas, 1983, J. Macromol. Sci. Rev. Macromol. Chem. 23:61; see also Levy et al., 1985, Science 228:190; During et al., 1989, Ann. Neurol. 25:351; Howard et al., 1989, J. Neurosurg. 71:105).

In another embodiment, a controlled-release system can be placed in proximity of the target area to be treated, thus requiring only a fraction of the systemic dose (see, e.g., Goodson, in Medical Applications of Controlled Release, supra, vol. 2, pp. 115-138 (1984)). Other controlled-release systems discussed in the review by Langer, 1990, Science 249:1527-1533) may be used.

Administration of any cancer therapy (and/or additional agents) described herein can, independently, be one to four times daily or one to four times per month or one to six times per year or once every two, three, four or five years. Administration can be for the duration of one day or one month, two months, three months, six months, one year, two years, three years, and may even be for the life of the subject.

The dosage regimen utilizing any cancer therapy (and/or additional agents) described herein can be selected in accordance with a variety of factors including type, species, age, weight, sex and medical condition of the subject; the severity of the condition to be treated; the route of administration; the renal or hepatic function of the subject; the pharmacogenomic makeup of the individual; and the specific compound of the invention employed. Any cancer therapy (and/or additional agents) described herein can be administered in a single daily dose, or the total daily dosage can be administered in divided doses of two, three or four times daily. Furthermore, any cancer therapy (and/or additional agents) described herein can be administered continuously rather than intermittently throughout the dosage regimen.

### EXAMPLES

The examples herein are provided to illustrate advantages and benefits of the present disclosure and to further assist a person of ordinary skill in the art with preparing or using cells that are resistant to anti-PD-1, anti-PD-L1 and/or anti-PD-L2 therapy. The examples herein are also presented in order to more fully illustrate the preferred aspects of the present disclosure. The examples should in no way be construed as limiting the scope of the present disclosure, as defined by the appended claims. The examples can include or incorporate any of the variations, aspects or embodiments of the present disclosure described above. The variations, aspects or embodiments described above may also further each include or incorporate the variations of any or all other variations, aspects or embodiments of the present disclosure.

### Example 1: Generation of Anti-PD-1-Resistant CT26 Tumors

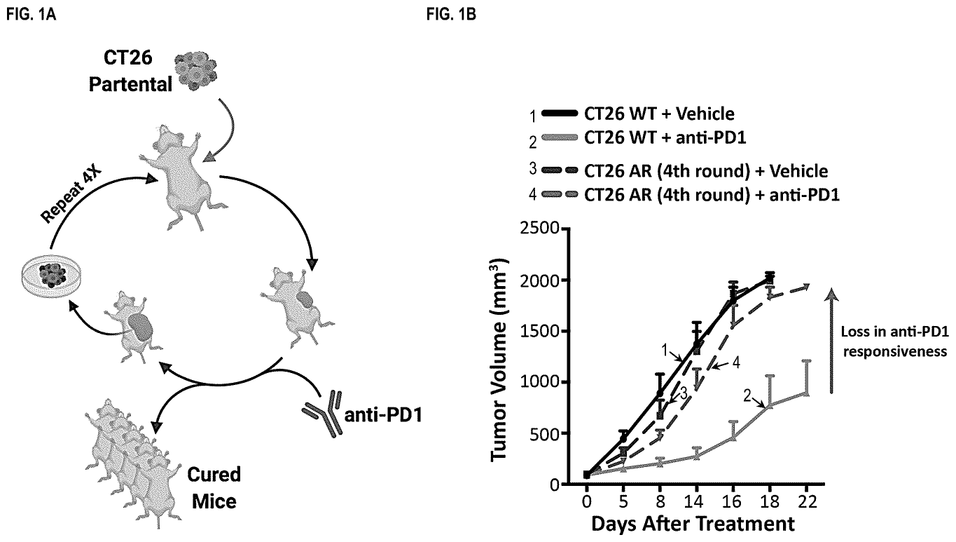

Murine colon carcinoma CT26 cell were subjected to selection for surviving the anti-PD-1 treatment to generate anti-PD-1-antibody resistant CT26 cells. The method used to generation of the anti-PD-1-resistant CT26 tumor cells is illustrated in FIG. 1A. Briefly, BALB/C mice were acquired from the Jackson Laboratory, and after several days of acclimation, mice were inoculated with 500,000 murine colon carcinoma CT26 cells on the rear flank. When average tumor volume reached 80-100 mm 3 (indicating day 0), mice were given a series of intraperitoneal injections of 100 μg anti-PD-1 (clone RMP1-14; BioXcell) on days 0, 3, and 6. Tumors were excised from mice that did not respond to anti-PD-1 therapy, approximately 10-14 days following the initial treatment. Tumors were dissociated using collagenase (StemCell Technologies), washed in 1×PBS, and plated in IMDM culture media supplemented with 10% fetal bovine serum, 1% GLUTiMAX, and 1% Antibiotic-Antimycotic (all GIBCO). Cells were passaged at least 5 times and were then inoculated into new recipient mice according as described above (round 2). Again, when tumors reached 80-100 mm³, another treatment course of anti-PD-1 was initiated: intraperitoneal injections of 100 μg anti-PD-1 (clone RMP1-14; BioXcell) were administered on days 0, 3, and 6. This process was repeated for a total of 4 rounds, at which point none of the treated mice responded to anti-PD-1 therapy. The cell lines generated after 2 rounds of anti-PD-1 selection are referred to throughout this disclosure as “2nd round,” “2nd generation,” or “F2 generation.” The cell lines generated after 4 rounds of anti-PD-1 selection are referred to as “4th round,” “4th generation,” or “F4 generation.”

The efficacy of anti-PD-1 antibody in CT26 cell- or anti-PD-1-antibody resistant CT26 cell-allografts was compared. Briefly, BALB/C mice were inoculated with CT26 parental cells and PD-1 resistant 4th generation cells in rear flanks. When the starting tumor volume (STV) reached 80-100 mm 3, mice were randomly divided in the following two treatment groups: (1) vehicle (PBS), and (2) anti-PD-1 antibody. Mice were given a series of intraperitoneal injections of vehicle or 100 μg anti-PD-1 (clone RMP1-14; BioXcell) on days 0, 3, and 6. Tumor volumes were measured on indicated days and plotted as a function of time. As shown in FIG. 1B, the growth of CT26 parental cell tumors was significantly retarded when treated with the anti-PD-1 antibody, compared to vehicle-only control (compare the grey solid line with the black solid line in FIG. 1B). In contrast, the PD-1 resistant cells showed very little retardation of tumor, if any. These results demonstrate, inter a/ia, that the anti-PD-1 resistance of anti-PD-1 resistant cells was developed after therapy (acquired resistance) in immune competent mice.

Therefore, anti-PD-1 antibody-resistant cells were developed. Also, a mouse model of cells harboring anti-PD-1 resistant cells was developed. Accordingly, the mouse model of disclosed herein is useful for testing an anti-cancer drug candidate by administering the anti-cancer drug candidate to mice bearing anti-PD-1-antibody resistant CT26 cell-allografts, and evaluating whether the anti-cancer drug candidate is effective in slowing or inhibiting cancer growth. An anti-cancer drug or candidate that is effective in slowing or inhibiting cancer growth may be formulated for administration to a human patient.

### Example 2: Transcriptomic Profiling of Anti-PD-1 Resistant Cell Lines Using RNA-Seq

Three distinct vials of parental CT26 cells (ATCC; “experimental replicates”), two independently isolated tumors from “2nd round” mice, four independently isolated tumors from “4th round” mice (both “biological replicates”) were cultured with or without 20 ng/mL of mouse IFNγ (Biolegend) for 24 hours at 37° 015%002. The following day, RNA was isolated from cells using Qiagen RNeasy reagents according to manufacturer's instructions, including QIASHREDDER homogenization and on-column DNaseI digestion. Isolated RNA was subjected to RNA-seq and data analysis. Briefly, sequencing libraries were generated and sequenced on an ILLUMINA HISEQ (2×150 paired end reads), targeting >20×10⁶ reads per sample. Sequences were trimmed using TRIMMOMATIC v.0.36 and mapped to the Mus musculus GRCm38 reference genome using the STAR ALIGNER v.2.5.2b. Unique gene hit counts were calculated by using FEATURECOUNTS from the SUBREAD package v.1.5.2. Only unique reads that fell within exon regions were counted. Differential gene expression was determined using DESeq2, and the Wald test was used to generate p-values and log 2 fold changes; with log 2 fold change >1 and adjusted p-values <0.05 as cutoffs for significance.

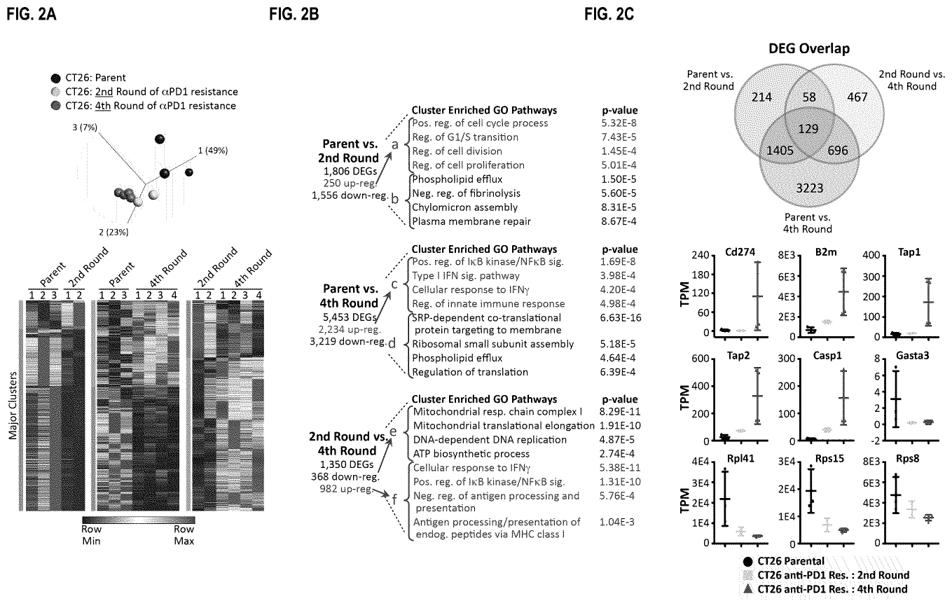

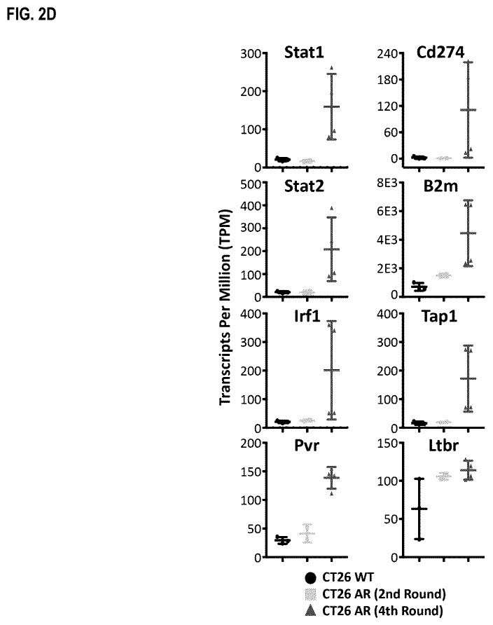

As shown in FIG. 2A (Top Panel), Principal Component Analysis (PCA) illustrated that that the samples could be spatially separated based on transcriptome expression. Differentially Expressed Genes (DEGs) were determined between the groups (parent vs. 2nd generation, parent vs. 4th generation, 2nd generation vs. 4th generation), and plotted in the heatmap. As shown in FIG. 2A (Bottom Panel), Hierarchical clustering performed to rank order genes on each row, separated genes into 2 major clusters in each comparison, where a subset of gene expression was lower (blue) in one dataset and higher (red) in the other. The genes were then subjected to analysis using the PANTHER application to identify Gene Ontologies (GO) associated with each gene set. Gene sets are shown with associated p-values. Up- or down-regulated genes were identified in each dataset. As shown in FIG. 2B, genes associated with the following GO were upregulated in 2nd generation cells compared to parental CT26 cells: positive regulation of cell cycle process, regulation of G1/S transition, regulation of cell division, and regulation of cell proliferation. Genes associated with the following GO were downregulated in 2nd generation cells compared to parental CT26 cells: phospholipid efflux, negative regulation of fibrinolysis, chylomicron assembly, and plasma membrane repair (FIG. 2B). Further, as shown in FIG. 2B, genes associated with the following GO were upregulated in 4th generation cells compared to parental CT26 cells: positive regulation of 103 kinase/NFκB signaling, type I IFN signaling pathway, cellular response to IFNγ, regulation of innate immune response. This was surprising, inter alia, because it has been widely known that cellular response to IFNγ is involved in sensitivity to anti-PD-1 and other immunotherapeutics. Genes associated with the following GO were downregulated in 4th generation cells compared to parental CT26 cells: SRP-dependent co-translational protein targeting to membrane, ribosomal small subunit assembly, phospholipid efflux, regulation of translation (FIG. 2B). Interestingly, the genes associated with the following GO were upregulated in 4th generation cells compared to 2nd generation cells: cellular response to IFNγ, positive regulation of IκB kinase/NFκB signaling, negative regulation of antigen processing/presentation, and antigen processing/presentation of endogenous peptides via MHC class I (FIG. 2B). Genes associated with the following GO were downregulated in 4th generation cells compared to 2nd generation cells: mitochondrial respiratory chain complex I, mitochondrial translational elongation, DNA-dependent DNA replication, and ATP biosynthetic process (FIG. 2B). A Venn diagram of overlap in gene expression between all datasets was created. As shown in FIG. 2C (Top Panel), the DEGs showed a significant overlap between 4th generation cells and 2nd generation cells. FIG. 2A (Bottom Panel) shows the transcripts per million (TPM; normalized expression data) at select genes, demonstrating higher baseline expression of genes associated with PD-L1, antigen processing/presentation, protein translation, ER trafficking; in some datasets over others. As shown in FIG. 2C (Bottom Panel), the genes Cd274, B2M, Tap1, Tap2, Casp1, and Gasta3 showed progressively increasing expression from CT26 parental cells to 2nd generation cells and to 4th generation cells. Further, genes Rpl41, Rps15 and Rps8 showed progressively decreasing expression from CT26 parental cells to 2nd generation cells and to 4th generation cells (FIG. 2C (Bottom Panel)). As shown in FIG. 2D, the genes Stat1, Stat2, Irf, Ltbr, and Pyr showed progressively increasing expression from CT26 parental cells to 2nd generation cells and to 4th generation cells.

These results, inter alia, establish biomarkers associated with acquired resistance to anti-PD-1 therapy. Such biomarkers may be used to identify patients that that may benefit from anti-PD-1 therapy or those that will not benefit from anti-PD-1 therapy. Therefore, based on these biomarkers, a patient may be selected for treatment with an anti-PD-1 therapy based on evaluating the sample for the presence, absence, or level of genes associated with one or more gene ontology (GO) pathways disclosed herein from a biological sample from the patient. A cancer therapy with an ability to inhibit function and/or activity of PD-1, PD-L1 and/or PD-L2 may be indicated, for example, when expression of one or more of Rpl41, Rps15 and Rps8 is high and/or the expression of one or more of Cd274, B2m, Tap1, Tap2, Casp1, and Gasta3 is low. In contrast, a cancer therapy with an ability to inhibit function and/or activity of PD-1, PD-L1 and/or PD-L2 may not be indicated, for example, when expression of one or more of Rpl41, Rps15 and Rps8 is low and/or the expression of one or more of Cd274, B2m, Tap1, Tap2, Casp1, and Gasta3 is high. The patients featuring low expression of one or more of Rpl41, Rps15 and Rps8 and/or high expression of one or more of Cd274, B2m, Tap1, Tap2, Casp1, and Gasta3 are likely to benefit from adjuvant or neoadjuvant therapies that eliminate the PD-1-nonresponsive cells.

### Example 3: Cell Surface Expression of PD-L1, PD-L2, MHC Class I and β2 Microglobulin in Anti-PD-1 Resistant CT26

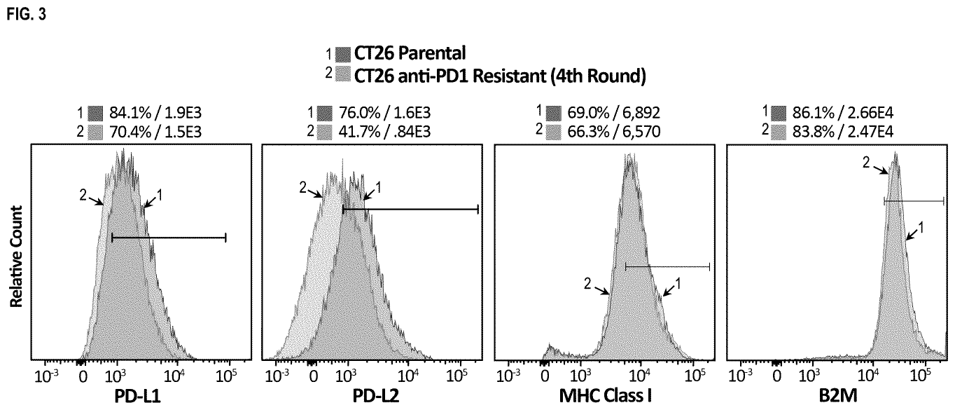

Since Cd274 (PD-L1), β2 microglobulin (B2m), and Tap1 and Tap2 genes (potentially involved in the processing and transport of major histocompatibility complex class I-associated antigen to the endoplasmic reticulum) were progressively downregulated from CT26 parental cells to 2nd generation cells and to 4th generation cells, surface expression of PD-L1, PD-L2, MHC Class I and β2 microglobulin (B2m) was studied. Briefly, parental and were harvested from culture and analyzed by flow cytometry for surface expression of PD-L1, PD-L2, MHC Class I, and β2 microglobulin (B2M). Gates were drawn as shown, and shown above each plot is the percentage of cells in each gate, and to the right of each percentage, the MFI (mean fluorescent intensity) of each marker. As shown in FIG. 3, the surface expression of PD-L1, MHC Class I and β2 microglobulin (B2M) was not downregulated in 4th generation anti-PD-1 resistant cells compared to parental cells. on the other hand, the surface expression of PD-L2 was downregulated in 4th generation anti-PD-1 resistant cells compared to parental cells. These data show that a discordance in gene expression and cell surface protein expression was observed surface expression in PD-L1/2 MHC Class I and B2M compared to RNA expression.

### Example 4: Transcriptomic Profiling of the Anti-PD-1 Resistant Cell Lines and a Primarily Resistant Anti-PD-1 Resistant Cell Line

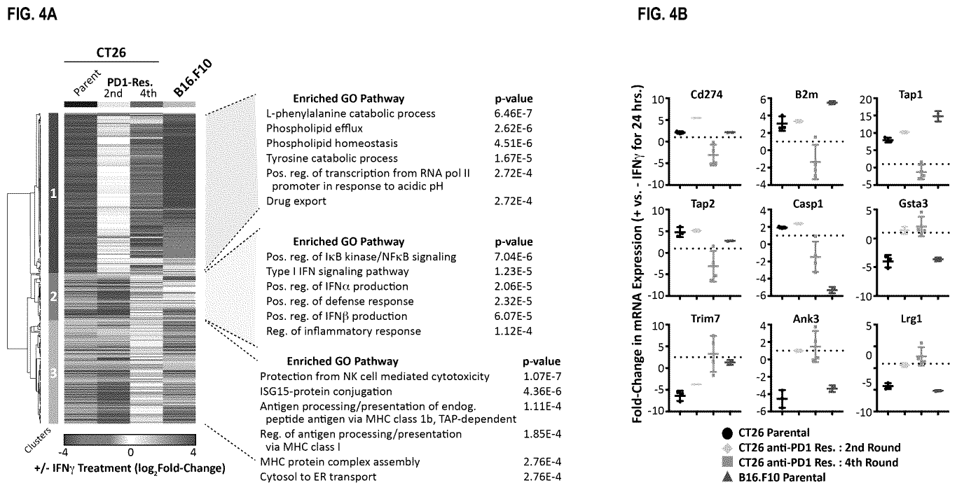

Next the transcriptomic profile of the 2nd and 4th generation anti-PD-1 resistant cell lines was studied by comparison with each other and with an anti-PD-1 primarily resistant cell line. The B16.F10 murine melanoma tumor cell line was used as a model of anti-PD-1 “primary resistance,” as these tumors are not responsive to anti-PD-1 therapy. Three distinct vials of parental CT26 cells (ATCC; “experimental replicates”), two independently isolated tumors from “2nd round” mice, four independently isolated tumors from “4th round” mice (both “biological replicates”), and two distinct vials of parental B16.F10 cells (ATCC; “experimental replicates were cultured with or without 20 ng/mL of mouse IFNγ (Biolegend) for 24 hours at 37° C./5% CO2 to assess in vitro responsiveness. This mimics how tumor cells respond in vivo, as immune cell infiltrate and secrete effector cytokines like IFNγ. The following day, RNA was isolated from cells using Qiagen RNeasy reagents according to manufacturers instructions, including QIASHREDDER homogenization and on-column DNaseI digestion. Isolated RNA was subjected to RNA-seq and data analysis. Briefly, sequencing libraries were generated and sequenced on an ILLUMINA HISEQ (2×150 paired end reads), targeting >20×10⁶ reads per sample. Sequences were trimmed using TRIMMOMATIC v.0.36 and mapped to the Mus musculus GRCm38 reference genome using the STAR ALIGNER v.2.5.2b. Unique gene hit counts were calculated by using FEATURECOUNTS from the SUBREAD package v.1.5.2. Only unique reads that fell within exon regions were counted. Differential gene expression was determined using DESeq2, and the Wald test was used to generate p-values and log 2 fold changes; with log 2 fold change >1 and adjusted p-values <0.05 as cutoffs for significance. The DEGs were identified between untreated and IFNγ treated parental CT26. As shown in FIG. 4A (Left Panel), Log 2 fold-change was plotted in the heatmap and genes are hierarchical clustered based on parental CT26. Of these, 338 genes had usable data from the other datasets; and those values are shown in the other columns (FIG. 4A (Left Panel)). Genes separated into 3 major clusters (FIG. 4A (Left Panel)). Associated genes were input into PANTHER to identify pathways associated with the dysregulated genes and the GO pathways associated with the DEGs were identified. As shown in FIG. 4A (Right Panel), the upregulation of expression of the genes associated with following GO pathways was enriched in 2nd generation cells and 4th generation cells compared to both parental CT26 cells and B16.F10 cells: L-phenyl alanine catabolic process, phospholipid efflux, tyrosine catabolic process, positive regulation of transcription from RNA polymerase II promoter in response to acidic pH, and drug export. Therefore, it is likely that upregulation of genes associated with one or more of those GO pathways is associated with acquired resistance to anti-PD-1 therapy. Further, the downregulation of expression of the genes associated with following GO pathways was observed in 4th generation cells compared to both parental CT26 cells and B16.F10 cells: protection of NK cell mediated cytotoxicity, IGS15-protein conjugation, antigen processing/presentation via MHC class I, MHC protein complex assembly, and cytosol to ER transport (FIG. 4A (Right Panel)). Therefore, it is likely that downregulation of one or more of those GO pathways is associated with acquired resistance to anti-PD-1 therapy. As shown in FIG. 4A (Right Panel), the downregulation of expression of the genes associated with following GO pathways was enriched in 4th generation cells and B16.F10 cells compared to both parental CT26 cells: positive regulation of IκB kinase/NFκB signaling, type I IFN signaling pathways, positive regulation of IFNα production, positive regulation of defense response, positive regulation of IFNβ production, and regulation of inflammatory response. Therefore, it is likely that downregulation of one or more of those GO pathways is associated with resistance to anti-PD-1 therapy. FIG. 4B shows the transcripts per million (TPM; normalized expression data) at select genes. As shown in FIG. 4B, although CT26 anti-PD-1 resistant cells have baseline hyperactivation of type I and type II interferons, when those cells are challenged with IFNγ, those cells downregulate these genes. This was surprising, inter alia, because it has been widely known that positive regulation of IκB kinase/NFκB signaling, type I IFN signaling pathways, positive regulation of IFNα production, positive regulation of defense response, positive regulation of IFNβ production, and regulation of inflammatory response are all involved in sensitivity to anti-PD-1 and other immunotherapeutics.

These results, inter alia, establish biomarkers associated with acquired resistance to anti-PD-1 therapy, and biomarkers associated with resistance to anti-PD-1 therapy (acquired or primary resistance). Such biomarkers may be used to identify patients that that may benefit from anti-PD-1 therapy or those that will not benefit from anti-PD-1 therapy. Therefore, based on these biomarkers, a patient may be selected for treatment with an anti-PD-1 therapy based on evaluating the sample for the presence, absence, or level of genes associated with one or more gene ontology (GO) pathways disclosed herein from a biological sample from the patient. A cancer therapy with an ability to inhibit function and/or activity of PD-1, PD-L1 and/or PD-L2 may be indicated, for example, when expression of Trim7 and/or Ank3 is high and/or the expression of Tap2, and/or Casp1 is low. In contrast, a cancer therapy with an ability to inhibit function and/or activity of PD-1, PD-L1 and/or PD-L2 may not be indicated, for example, when expression of Trim7 and/or Ank3 is high and/or the expression of Tap2, and/or Casp1 is low. The patients featuring when high expression of Trim7 and/or Ank3, and/or the low expression of Tap2, and/or Casp1 is are likely to benefit from adjuvant or neoadjuvant therapies that eliminate the PD-1-nonresponsive cells.

### Example 5: Paradoxical Dysregulation of Genes that are Differentially Regulated in Anti-PD-1 Resistant Cells Compared to Parental Cells

Next, the effect of IFNγ on the expression of certain genes that are differentially expressed in the 4th generation anti-PD-1 resistant cells compared to the wild type CT26 cells was studied. Briefly, the parental CT26 cells, second generation anti-PD-1 resistant cells and 4th generation anti-PD-1 resistant cells were cultured with or without 20 ng/mL of mouse IFNγ (Biolegend) for 24 hours at 37° C./5%002. The following day, RNA was isolated from cells using Qiagen RNeasy reagents according to manufacturer's instructions, including QIASHREDDER homogenization and on-column DNaseI digestion. Isolated RNA was subjected to RNA-seq and data analysis. Briefly, sequencing libraries were generated and sequenced on an ILLUMINA HISEQ (2×150 paired end reads), targeting >20×10⁶ reads per sample. Sequences were trimmed using TRIMMOMATIC v.0.36 and mapped to the Mus musculus GRCm38 reference genome using the STAR ALIGNER v.2.5.2b. Unique gene hit counts were calculated by using FEATURECOUNTS from the SUBREAD package v.1.5.2. Only unique reads that fell within exon regions were counted. Differential gene expression was determined using DESeq2, and the Wald test was used to generate p-values and log 2 fold changes; with log 2 fold change >1 and adjusted p-values <0.05 as cutoffs for significance.

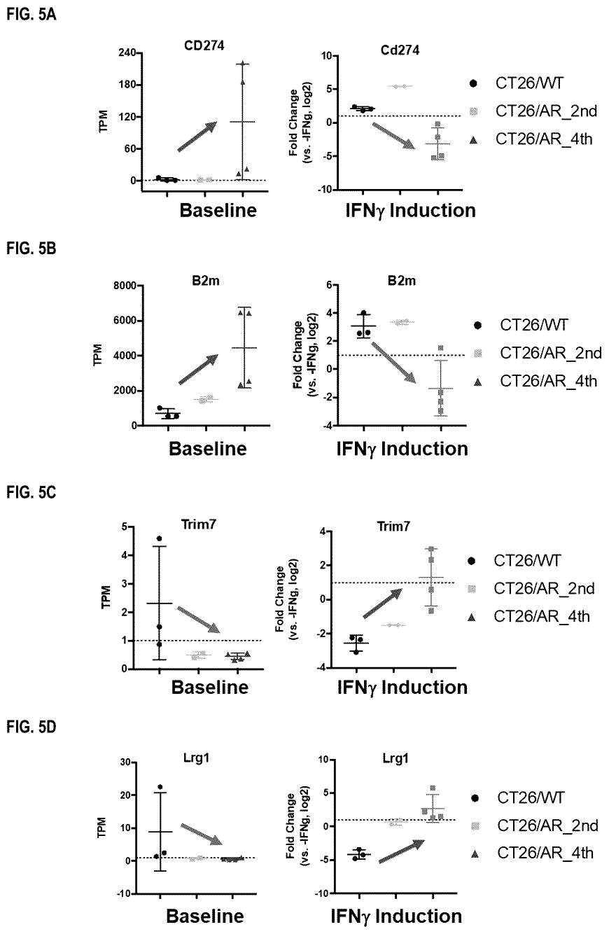

To understand the effect of IFNγ, the transcripts per million (TPM; normalized expression data) of representative genes that are either overexpressed (Cd274 and B2m) or repressed (Trim7 and Lrg1) were analyzed. As expected, the expression of Cd274 (PD-L1) increased from wild type CT26 cells to second generation anti-PD-1 resistant cells and to 4th generation anti-PD-1 resistant cells (FIG. 5A (left panel)). The expression of Cd274 increased in wild type CT26 cells increased when the wild type CT26 cells were induced with IFNγ (compare closed circles data in FIG. 5A (left and right panels)). Paradoxically, the expression of Cd274 (PD-L1) decreased from wild type CT26 cells to second generation anti-PD-1 resistant cells and to 4th generation anti-PD-1 resistant cells in response to IFNγ (FIG. 5A (right panel)).

The expression of B2M also increased from wild type CT26 cells to second generation anti-PD-1 resistant cells and to 4th generation anti-PD-1 resistant cells, as expected (FIG. 5B (left panel)). The expression of B2M increased in wild type CT26 cells increased when the wild type CT26 cells were induced with IFNγ (compare closed circles data in FIG. 5B (left and right panels)). Paradoxically, the expression of B2M decreased from wild type CT26 cells to second generation anti-PD-1 resistant cells and to 4th generation anti-PD-1 resistant cells in response to IFNγ (FIG. 5B (right panel)).

As expected, the expression of Trim7 decreased from wild type CT26 cells to second generation anti-PD-1 resistant cells and to 4th generation anti-PD-1 resistant cells (FIG. 5C (left panel)). The expression of Trim7 increased in wild type CT26 cells increased when the wild type CT26 cells were induced with IFNγ (compare closed circles data in FIG. 5C (left and right panels)). Paradoxically, the expression of Trim7 decreased from wild type CT26 cells to second generation anti-PD-1 resistant cells and to 4th generation anti-PD-1 resistant cells in response to IFNγ (FIG. 5C (right panel)).

Similarly, the expression of Lrg1 decreased from wild type CT26 cells to second generation anti-PD-1 resistant cells and to 4th generation anti-PD-1 resistant cells (FIG. 5D (left panel)). The expression of Lrg1 increased in wild type CT26 cells increased when the wild type CT26 cells were induced with IFNγ (compare closed circles data in FIG. 5D (left and right panels)). Paradoxically, the expression of Lrg1 decreased from wild type CT26 cells to second generation anti-PD-1 resistant cells and to 4th generation anti-PD-1 resistant cells in response to IFNγ (FIG. 5D (right panel)).

These results suggest, inter alia, a paradoxical dysregulation of genes like Cd274, B2m, Trim7 and Lrg1 compared to parental cancer cells when grown in the presence or absence of IFNγ.

### Example 6: Identifying Driver Genes of Resistance

Since the dysregulation was observed with multiple genes, without being bound by theory, it was hypothesized that the paradoxical dysregulation is the functional consequence of the acquired resistance to anti-PD-1 agents. To identify the driver genes involved in the observed paradoxical dysregulation, the analysis disclosed in FIG. 6A was conducted. Briefly, the Differentially Expressed Genes (DEGs) in 4th generation anti-PD-1 resistant cells compared to parental CT 26 cells when grown in the presence of IFNγ were identified. As shown in FIG. 6A (Panel 1), 1,999 genes are downregulated and 3607 genes are upregulated the 4th generation anti-PD-1 resistant cells in the presence of IFNγ compared to the CT26 cells. From these genes, those that were downregulated the 4th generation anti-PD-1 resistant cells but were upregulated the CT26 cells were identified. As shown in FIG. 6A (Panel 2), 1,060 genes were narrowed using this criterion (downregulated the 4th generation anti-PD-1 resistant cells but are upregulated the CT26 cells. these genes were further narrowed based on responstiveness to IFNγ. As shown in FIG. 6A (Panel 3), 688 genes that are downregulated the 4th generation anti-PD-1 resistant cells compared to the CT26 cells as revealed by sorting according to responstiveness to IFNγ. These genes include Lrg1, Spry2, Arg1, Trim8, Trim2, Mapk8ip1, Trim7, Trim6, etc. (FIG. 6A (Panel 3)).

Then to further narrow these genes, the parental CT26 cells, second generation anti-PD-1 resistant cells and 4th generation anti-PD-1 resistant cells were grown in vivo in mice. RNA was isolated from tumors and subjected to RNA-seq and data analysis. From the 688 genes from (FIG. 6A (Panel 3). Those genes that were also upregulated in vivo were identified. As shown in FIG. 6A (Panel 4), 70 genes were upregulated in vivo in the 4th generation anti-PD-1 resistant cells compared to the CT26 cells. these genes included Krt8, Mapk8ip1, Arg1 and Lrg1. FIG. 6B shows the gene ontology (GO) pathways that were enriched in at least two steps in FIG. 6A. This analysis identified TRIM family of proteins (especially, Trim7), Mapk8ip1, Elk1, Lrg1, Arg1, to be some of the drivers. FIG. 6C shows the functional pathways affected by the TRIM family of proteins. FIG. 6D shows the functional pathways in which Elk1 and c-Jun play a role. FIG. 6E shows the functional connections between Lrg1, B2m and Arg1 with other genes. FIG. 6F shows the levels of expression of Elk1 in tumors and surrounding normal tissue in the Cancer Genome Atlas (TOGA) cancer genomics program.

### Example 7: Paradoxical Dysregulation of Additional Differentially Regulated Genes

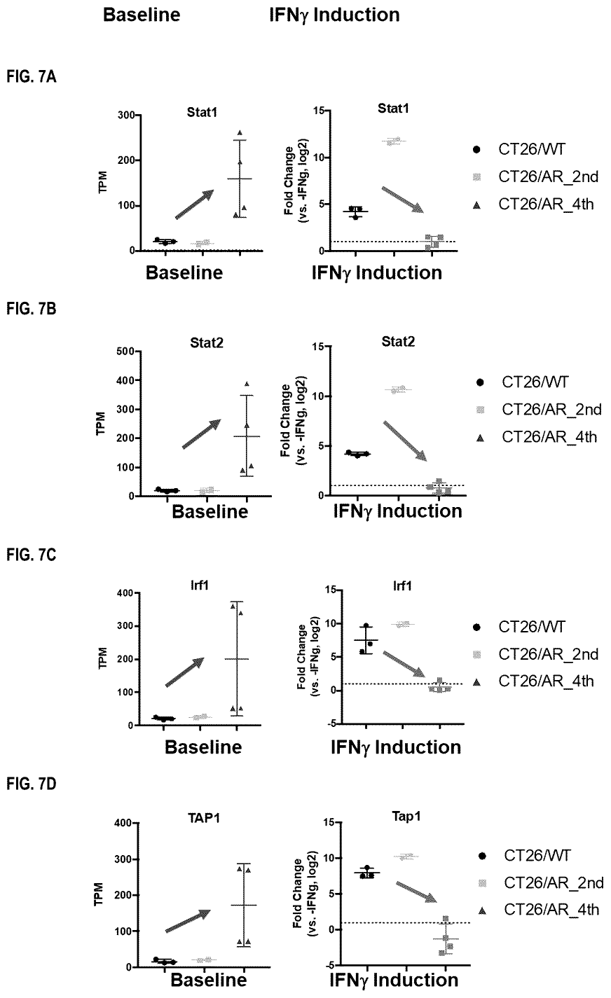

Transcripts per million (TPM; normalized expression data) for additional representative genes that are overexpressed (Stat1, Stat2, Irf1 and Tap1) were analyzed. The expression of Stat1 increased from wild type CT26 cells to second generation anti-PD-1 resistant cells and to 4th generation anti-PD-1 resistant cells (FIG. 7A (left panel)). The expression of Stat1 increased in wild type CT26 cells increased when the wild type CT26 cells were induced with IFNγ (compare closed circles data in FIG. 7A (left and right panels)). Paradoxically, the expression of Stat1 decreased from wild type CT26 cells to second generation anti-PD-1 resistant cells and to 4th generation anti-PD-1 resistant cells in response to IFNγ (FIG. 7A (right panel)).

The expression of Stat2 also increased from wild type CT26 cells to second generation anti-PD-1 resistant cells and to 4th generation anti-PD-1 resistant cells (FIG. 7B (left panel)). The expression of Stat2 increased in wild type CT26 cells increased when the wild type CT26 cells were induced with IFNγ (compare closed circles data in FIG. 7B (left and right panels)). Paradoxically, the expression of Stat2 decreased from wild type CT26 cells to second generation anti-PD-1 resistant cells and to 4th generation anti-PD-1 resistant cells in response to IFNγ (FIG. 7B (right panel)).

Similarly, the expression of Irf1 increased from wild type CT26 cells to second generation anti-PD-1 resistant cells and to 4th generation anti-PD-1 resistant cells (FIG. 7C (left panel)). The expression of Irf1 increased in wild type CT26 cells increased when the wild type CT26 cells were induced with IFNγ (compare closed circles data in FIG. 7C (left and right panels)). Paradoxically, the expression of Irf1 decreased from wild type CT26 cells to second generation anti-PD-1 resistant cells and to 4th generation anti-PD-1 resistant cells in response to IFNγ (FIG. 7C (right panel)).

As expected, the expression of Tap1 increased from wild type CT26 cells to second generation anti-PD-1 resistant cells and to 4th generation anti-PD-1 resistant cells (FIG. 7D (left panel)). The expression of Tap1 increased in wild type CT26 cells increased when the wild type CT26 cells were induced with IFNγ (compare closed circles data in FIG. 7D (left and right panels)). Paradoxically, the expression of Tap1 decreased from wild type CT26 cells to second generation anti-PD-1 resistant cells and to 4th generation anti-PD-1 resistant cells in response to IFNγ (FIG. 7D (right panel)).

### Example 8: Pathway Analysis of the Differentially Regulated Genes

To perform pathway analysis was performed using the WEB-based GEne SeT AnaLysis Toolkit (WebGestalt). Briefly, top 1,000 genes that were downregulated the 4th generation anti-PD-1 resistant cells but were upregulated the CT26 cells were (see FIG. 6A (Panel 2)), were rank ordered based on the fold-difference in expression between the 4th generation anti-PD-1 resistant cells and the parental CT-26 samples. These genes and rankings were input into the WebGestalt to identify enriched/de-enriched pathways associated with these genes, using gene set enrichment analysis (GSEA) and the Kyoto Encyclopedia of Genes and Genomes (KEGG) pathway functional database. Benjamini-Hochberg false discovery rate (FDR) was used for statistical significance. This analysis showed that the following pathways were overactive in the 4th generation anti-PD-1 resistant cells compared to the parental CT-26 cells: circardian entrainment, cAMP signaling pathway, cholinergic synapse, melanogenesis, Rap1 signaling pathway, human cytomegalovirus infection, human immunodeficiency 1 virus infection, glutamatergic synapse, pathways in cancer and Ras signaling pathway (FIG. 8A). The following pathways were repressed in in the 4th generation anti-PD-1 resistant cells compared to the parental CT-26 cells: systematic lupus erythematosus, microRNAs in cancer, hepatocellular carcinoma, tuberculosis and protein processing in endoplasmic reticulum. To visualize of the enrichment score and significance, a volcano plat was prepared based on the data presented in FIG. 8A. FIG. 8B shows the volcano plot. As shown in FIG. 8B, although circadian entrainment was the farthest ‘dot’ to the right, pathways in cancer and Ras signaling were the highest up (i.e. having the most significant FDR values). Although there wasn't strong significance (All FDR>0.05, which often happens in the noise of genomics), Ras/Rapt signaling pathways were identified in this unbiased analysis. FIG. 8C shows the RAS signaling pathway, illustrating the convergence with Raf/Mek/Erk signaling. FIG. 8D shows the RAP1 signaling pathway, illustrating the convergence with Raf/Mek/Erk signaling.

### Example 9: Paradoxical Dysregulation of Ccl5 (RANTES), Cxcl10 (IP-10) and Ifnb1

Nest transcripts per million (TPM; normalized expression data) for Ccl5 (RANTES), Cxcl10 (IP-10) and Ifnb1 were analyzed. These genes are induced by the activation of Trim7.

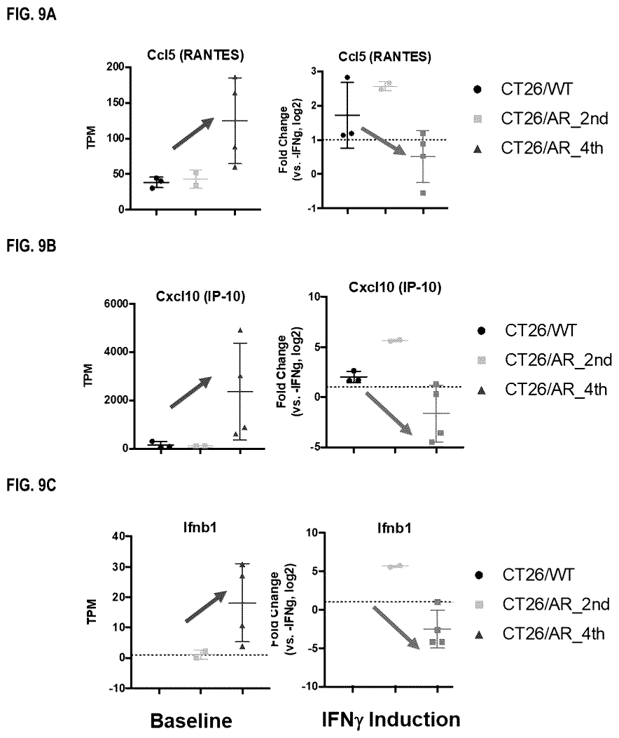

Ccl5 (RANTES) and Cxcl10 (IP-10) are among genes that are regulated by IFNγ. The promoters regulating Ccl5 (RANTES) and Cxcl10 (IP-10) contain binding sites for STAT1 dimers and/or ISGF3. As expected, the expression of Ccl5 (RANTES) increased from wild type CT26 cells to second generation anti-PD-1 resistant cells and to 4th generation anti-PD-1 resistant cells (FIG. 9A (left panel)). The expression of Ccl5 (RANTES) increased in wild type CT26 cells increased when the wild type CT26 cells were induced with IFNγ (compare closed circles data in FIG. 9A (left and right panels)). Paradoxically, the expression of Ccl5 (RANTES) decreased from wild type CT26 cells to second generation anti-PD-1 resistant cells and to 4th generation anti-PD-1 resistant cells in response to IFNγ (FIG. 9A (right panel)).

The expression of Cxcl10 (IP-10) increased from wild type CT26 cells to second generation anti-PD-1 resistant cells and to 4th generation anti-PD-1 resistant cells, as expected (FIG. 9B (left panel)). The expression of Cxcl10 (IP-10) increased in wild type CT26 cells increased when the wild type CT26 cells were induced with IFNγ (compare closed circles data in FIG. 9B (left and right panels)). Paradoxically, the expression of Cxcl10 (IP-10) decreased from wild type CT26 cells to second generation anti-PD-1 resistant cells and to 4th generation anti-PD-1 resistant cells in response to IFNγ (FIG. 9B (right panel)).

The expression of Ifnb1 also increased from wild type CT26 cells to second generation anti-PD-1 resistant cells and to 4th generation anti-PD-1 resistant cells (FIG. 9C (left panel)). The expression of Ifnb1 increased in wild type CT26 cells increased when the wild type CT26 cells were induced with IFNγ (compare closed circles data in FIG. 9C (left and right panels)). Paradoxically, the expression of Ifnb1 decreased from wild type CT26 cells to second generation anti-PD-1 resistant cells and to 4th generation anti-PD-1 resistant cells in response to IFNγ (FIG. 9C (right panel)).

These results illustrate, inter alia, that acquired resistance to anti-PD-1 is associated with a paradoxical regulation of Trim7-regulated genes such as Ccl5 (RANTES), Cxcl10 (IP-10) and Ifnb1. Accordingly, these results suggest that anti-PD-1 resistant tumors may be treated with Trim7 modulators, e.g., Trim 7 inhibitors.

### INCORPORATION BY REFERENCE

All patents and publications referenced herein are hereby incorporated by reference in their entireties.

The publications discussed herein are provided solely for their disclosure prior to the filing date of the present application. Nothing herein is to be construed as an admission that the present disclosure is not entitled to antedate such publication by virtue of prior invention.

As used herein, all headings are simply for organization and are not intended to limit the disclosure in any manner. The content of any individual section may be equally applicable to all sections.

### EQUIVALENTS

While the invention has been disclosed in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

Those skilled in the art will recognize, or be able to ascertain, using no more than routine experimentation, numerous equivalents to the specific embodiments disclosed specifically herein. Such equivalents are intended to be encompassed in the scope of the following claims.

What is claimed is:

1. A method of determining a cancer treatment for a patient, the method comprising:
(a) obtaining a biological sample from a subject;
(b) evaluating the sample for the presence, absence, or level of one or more genes associated with a gene ontology (GO) pathway selected from:
(i) positive regulation of cell cycle process, regulation of G1/S transition, regulation of cell division, regulation of cell proliferation, positive regulation of IκB kinase/NFκB signaling, type I IFN signaling pathway, cellular response to IFNγ, positive regulation of IFNα production, positive regulation of defense response, positive regulation of IFNβ production, regulation of inflammatory response, regulation of innate immune response, negative regulation of antigen processing/presentation, and antigen processing/presentation of endogenous peptides via MHC class I; and/or
(ii) phospholipid efflux, negative regulation of fibrinolysis, chylomicron assembly, plasma membrane repair, SRP-dependent co-translational protein targeting to membrane, ribosomal small subunit assembly, phospholipid efflux, regulation of translation, mitochondrial respiratory chain complex I, mitochondrial translational elongation, DNA-dependent DNA replication, and ATP biosynthetic process;
and
(c) selecting the cancer therapy with an ability to inhibit function and/or activity of PD-1, PD-L1 and/or PD-L2 based on the evaluation of step (b).

2. A method for selecting a patient for a cancer treatment, the method comprising:
(a) obtaining a biological sample from a subject;
(b) evaluating the sample for the presence, absence, or level of one or more genes associated with a gene ontology (GO) pathway selected from:
(i) positive regulation of cell cycle process, regulation of G1/S transition, regulation of cell division, regulation of cell proliferation, positive regulation of IκB kinase/NFκB signaling, type I IFN signaling pathway, cellular response to IFNγ, positive regulation of IFNα production, positive regulation of defense response, positive regulation of IFNβ production, regulation of inflammatory response, regulation of innate immune response, negative regulation of antigen processing/presentation, and antigen processing/presentation of endogenous peptides via MHC class I; and/or
(ii) phospholipid efflux, negative regulation of fibrinolysis, chylomicron assembly, plasma membrane repair, SRP-dependent co-translational protein targeting to membrane, ribosomal small subunit assembly, phospholipid efflux, regulation of translation, mitochondrial respiratory chain complex I, mitochondrial translational elongation, DNA-dependent DNA replication, and ATP biosynthetic process;
and
(c) selecting a cancer therapy with an ability to inhibit function and/or activity of PD-1, PD-L1 and/or PD-L2.

3. A method of treating cancer, the method comprising:
(a) obtaining a biological sample from a subject;
(b) evaluating the sample for the presence, absence, or level of one or more genes associated with a gene ontology (GO) pathway selected from:
(i) positive regulation of cell cycle process, regulation of G1/S transition, regulation of cell division, regulation of cell proliferation, positive regulation of IκB kinase/NFκB signaling, type I IFN signaling pathway, cellular response to IFNγ, positive regulation of IFNα production, positive regulation of defense response, positive regulation of IFNβ production, regulation of inflammatory response, regulation of innate immune response, negative regulation of antigen processing/presentation, and antigen processing/presentation of endogenous peptides via MHC class I; and/or
(ii) phospholipid efflux, negative regulation of fibrinolysis, chylomicron assembly, plasma membrane repair, SRP-dependent co-translational protein targeting to membrane, ribosomal small subunit assembly, phospholipid efflux, regulation of translation, mitochondrial respiratory chain complex I, mitochondrial translational elongation, DNA-dependent DNA replication, and ATP biosynthetic process;
and
(c) selecting the cancer therapy selected from:
(i) an agent with an ability to inhibit function and/or activity of PD-1, PD-L1 and/or PD-L2;
(ii) an antimetabolite chemotherapeutic (e.g., 5-fluorouracil, methotrexate, capecitabine, azacitidine, 6-diazo-5-oxo-L-norleucine (DON), azaserine and acivicin), a topoisomerase inhibitor (e.g., irinotecan, topotecan, etoposide, doxorubicin, etc.); and
(iii) a protein translation inhibitor (e.g., silvestrol and omacetaxine) ribosome biogenesis inhibitors (e.g., diazaborine, lamotrigine and ribozinoindoles), inhibitors of rRNA and/or tRNA synthesis (e.g., quarfloxin (CX-3543) and CX-5461), an inhibitor of synthesis of amino acids (e.g., GLUD1 inhibitor R162, BCAT1 inhibitor gabapentin, glutaminase inhibitor bis-2-(5-phenylacetamido-1,2,4-thiadiazol-2-yl)ethyl sulfide (BPTES), PAGDH inhibitor NCT-503), an inhibitor of uptake of amino acids (e.g., SLC7A11 inhibitors sulfasalazine, erastin or sorafenib), a modulator of post-translational modification (e.g., glycosylation inhibitor tunicamycin, ppGalNAc-T3), a modulator of protein degradation, and a modulator of protein transport (e.g., cyclosporin A, fendiline, parbendazole, paroxetine, parthenolide, quinacrine, sertraline, spiperone, thimerosal, astemizole, perhexiline, HUN-7293, CAM741, CK147, and cotransin, etc.) or a topoisomerase inhibitor; and
(d) administering the cancer therapy with an ability to inhibit function and/or activity of PD-1, PD-L1 and/or PD-L2, and optionally administering the therapy selected in step (c)(ii) and (c)(iii).

4. The method of any one of claims 1 to 3, wherein an upregulation of one or more genes associated with a GO pathway listed in (i) compared to a healthy tissue indicates a lack of response, resistance or recalcitrance to the cancer therapy with an ability to inhibit function and/or activity of PD-1, PD-L1 and/or PD-L2.

5. The method of any one of claims 1 to 4, wherein an upregulation of one or more genes associated with a GO pathway listed in (i) compared to another biological sample from patient that is known to be sensitive to anti-PD-1 therapy indicates a lack of response, resistance or recalcitrance to the cancer therapy with an ability to inhibit function and/or activity of PD-1, PD-L1 and/or PD-L2.

6. The method of any one of claims 1 to 5, wherein an upregulation of one or more genes associated with a GO pathway listed in (i) compared to a prior biological sample obtained from the subject indicates a development of resistance, a lack of response, or recalcitrance to the cancer therapy with an ability to inhibit function and/or activity of PD-1, PD-L1 and/or PD-L2.

7. The method of any one of claims 1 to 3, wherein a lack of upregulation of one or more genes associated with a GO pathway listed in (i) compared to a healthy tissue indicates a response to the cancer therapy with an ability to inhibit function and/or activity of PD-1, PD-L1 and/or PD-L2.

8. The method of any one of claim 1 to 3 or 7, wherein a lack of upregulation of one or more genes associated with a GO pathway listed in (i) compared to another biological sample from patient that is known to be sensitive to anti-PD-1 therapy indicates a response to the cancer therapy with an ability to inhibit function and/or activity of PD-1, PD-L1 and/or PD-L2.

9. The method of any one of claim 1 to 3, 7, or 8 wherein a lack of upregulation of one or more genes associated with a GO pathway listed in (i) compared to a prior biological sample obtained from the subject indicates a response to the cancer therapy with an ability to inhibit function and/or activity of PD-1, PD-L1 and/or PD-L2.

10. The method of any one of claims 1 to 6, wherein a downregulation of one or more genes associated with a GO pathway listed in (ii) compared to a healthy tissue indicates a lack of response, resistance or recalcitrance to the cancer therapy with an ability to inhibit function and/or activity of PD-1, PD-L1 and/or PD-L2.

11. The method of any one of claim 1 to 6 or 10, wherein a downregulation of one or more genes associated with a GO pathway listed in (ii) compared to another biological sample from patient that is known to be sensitive to anti-PD-1 therapy indicates a lack of response, resistance or recalcitrance to the cancer therapy with an ability to inhibit function and/or activity of PD-1, PD-L1 and/or PD-L2.

12. The method of any one of claim 1 to 6, 10 or 11, wherein a downregulation of one or more genes associated with a GO pathway listed in (ii) compared to a prior biological sample obtained from the subject indicates a development of resistance, a lack of response, or recalcitrance to the cancer therapy with an ability to inhibit function and/or activity of PD-1, PD-L1 and/or PD-L2.

13. The method of any one of claims 1 to 3, or 7 to 9, wherein a lack of downregulation of one or more genes associated with a GO pathway listed in (ii) compared to a healthy tissue indicates a response to the cancer therapy with an ability to inhibit function and/or activity of PD-1, PD-L1 and/or PD-L2.

14. The method of any one of claim 1 to 3, 7 to 9, or 13, wherein a lack of downregulation of one or more genes associated with a GO pathway listed in (ii) compared to another biological sample from patient that is known to be sensitive to anti-PD-1 therapy indicates a response to the cancer therapy with an ability to inhibit function and/or activity of PD-1, PD-L1 and/or PD-L2.

15. The method of any one of claim 1 to 3, 7 to 9, 13 or 14, wherein a lack of downregulation of one or more genes associated with a GO pathway listed in (ii) compared to a prior biological sample obtained from the subject indicates a response to the cancer therapy with an ability to inhibit function and/or activity of PD-1, PD-L1 and/or PD-L2.

16. The method of any one of claims 1 to 3, 7 to 9, or 13 to 15, wherein a lack of upregulation of one or more genes associated with a GO pathway compared to a prior biological sample from the subject indicates a response to the cancer therapy with an ability to inhibit function and/or activity of PD-1, PD-L1 and/or PD-L2, wherein the GO pathway is selected from (a) cellular response to IFNγ, (b) negative regulation of antigen processing/presentation, (c) type I IFN signaling pathway, (d) positive regulation of IκB kinase/NFκB signaling, and antigen processing, and (e) presentation of endogenous peptides via MHC class I.

17. The method of any one of claims 1 to 3, 7 to 9, or 13 to 16, wherein a lack of upregulation of one or more genes associated with a GO pathway compared to a patient that is known to be sensitive to anti-PD-1 therapy indicates a response to the cancer therapy with an ability to inhibit function and/or activity of PD-1, PD-L1 and/or PD-L2, wherein the GO pathway is selected from (a) cellular response to IFNγ, (b) negative regulation of antigen processing/presentation, (c) type I IFN signaling pathway, (d) positive regulation of IκB kinase/NFκB signaling, and antigen processing, and (e) presentation of endogenous peptides via MHC class I.

18. The method of any one of claims 1 to 3, 7 to 9, or 13 to 17, wherein a lack of upregulation of one or more genes associated with a GO pathway compared to a standard indicates a response to the cancer therapy with an ability to inhibit function and/or activity of PD-1, PD-L1 and/or PD-L2, wherein the GO pathway is selected from (a) cellular response to IFNγ, (b) negative regulation of antigen processing/presentation, (c) type I IFN signaling pathway, (d) positive regulation of IκB kinase/NFκB signaling, and antigen processing, and (e) presentation of endogenous peptides via MHC class I.

19. The method of any one of claims 1 to 6, or 10 to 12, wherein an upregulation of one or more genes associated with a GO pathway compared to a prior biological sample from the subject indicates resistance, a lack of response, or recalcitrance to the cancer therapy with an ability to inhibit function and/or activity of PD-1, PD-L1 and/or PD-L2, wherein the GO pathway is selected from (a) cellular response to IFNγ, (b) negative regulation of antigen processing/presentation, (c) type I IFN signaling pathway, (d) positive regulation of IκB kinase/NFκB signaling, and antigen processing, and (e) presentation of endogenous peptides via MHC class I.

20. The method of any one of claim 1 to 6, 10 to 12 or 19, wherein an upregulation of one or more genes associated with a GO pathway compared to a patient that is known to be sensitive to anti-PD-1 therapy indicates resistance, a lack of response, or recalcitrance to the cancer therapy with an ability to inhibit function and/or activity of PD-1, PD-L1 and/or PD-L2, wherein the GO pathway is selected from (a) cellular response to IFNγ, (b) negative regulation of antigen processing/presentation, (c) type I IFN signaling pathway, (d) positive regulation of IκB kinase/NFκB signaling, and antigen processing, and (e) presentation of endogenous peptides via MHC class I.

21. The method of any one of claim 1 to 6, 10 to 12, 19 or 20, wherein an upregulation of one or more genes associated with a GO pathway compared to a standard indicates resistance, a lack of response, or recalcitrance to the cancer therapy with an ability to inhibit function and/or activity of PD-1, PD-L1 and/or PD-L2, wherein the GO pathway is selected from (a) cellular response to IFNγ, (b) negative regulation of antigen processing/presentation, (c) type I IFN signaling pathway, (d) positive regulation of IκB kinase/NFκB signaling, and antigen processing, and (e) presentation of endogenous peptides via MHC class I.

22. The method of any one of claims 1 to 3, 7 to 9, or 13 to 18, wherein a lack of upregulation of one or more genes associated with cellular response to IFNγ compared to a prior biological sample from the subject indicates a response to the cancer therapy with an ability to inhibit function and/or activity of PD-1, PD-L1 and/or PD-L2.

23. The method of any one of claim 1 to 3, 7 to 9, 13 to 18, or 22, wherein a lack of upregulation of one or more genes associated with cellular response to IFNγ compared to a patient that is known to be sensitive to anti-PD-1 therapy indicates a response to the cancer therapy with an ability to inhibit function and/or activity of PD-1, PD-L1 and/or PD-L2.

24. The method of any one of claim 1 to 3, 7 to 9, 13 to 18, 22 or 23, wherein a lack of upregulation of one or more genes associated with cellular response to IFNγ compared to a standard indicates a response to the cancer therapy with an ability to inhibit function and/or activity of PD-1, PD-L1 and/or PD-L2.

25. The method of any one of claims 1 to 6, 10 to 12, or 19 to 21, wherein an upregulation of one or more genes associated with cellular response to IFNγ compared to a prior biological sample from the subject indicates resistance, a lack of response, or recalcitrance to the cancer therapy with an ability to inhibit function and/or activity of PD-1, PD-L1 and/or PD-L2.

26. The method of any one of claim 1 to 6, 10 to 12, 19 to 21 or 25, wherein an upregulation of one or more genes associated with cellular response to IFNγ compared to a patient that is known to be sensitive to anti-PD-1 therapy indicates resistance, a lack of response, or recalcitrance to the cancer therapy with an ability to inhibit function and/or activity of PD-1, PD-L1 and/or PD-L2.

27. The method of any one of claim 1 to 6, 10 to 12, 19 to 21, 25 or 26, wherein an upregulation of one or more genes associated with cellular response to IFNγ compared to a standard indicates resistance, a lack of response, or recalcitrance to the cancer therapy with an ability to inhibit function and/or activity of PD-1, PD-L1 and/or PD-L2.

28. The method of any one of claims 1 to 6, 10 to 12, 19 to 21, or 25 to 27, wherein an upregulation of one or more genes associated with type I IFN signaling pathway compared to a prior biological sample from the subject indicates resistance, a lack of response, or recalcitrance to the cancer therapy with an ability to inhibit function and/or activity of PD-1, PD-L1 and/or PD-L2.

29. The method of any one of claims 1 to 6, 10 to 12, 19 to 21 or 25 to 28, wherein an upregulation of one or more genes associated with type I IFN signaling pathway compared to a patient that is known to be sensitive to anti-PD-1 therapy indicates resistance, a lack of response, or recalcitrance to the cancer therapy with an ability to inhibit function and/or activity of PD-1, PD-L1 and/or PD-L2.

30. The method of any one of claims 1 to 6, 10 to 12, 19 to 21, or 25 to 29, wherein an upregulation of one or more genes associated with type I IFN signaling pathway compared to a standard indicates resistance, a lack of response, or recalcitrance to the cancer therapy with an ability to inhibit function and/or activity of PD-1, PD-L1 and/or PD-L2.

31. The method of any one of claims 1 to 3, 7 to 9, 13 to 18, or 22 to 24, wherein a lack of upregulation of one or more genes associated with type I IFN signaling pathway compared to a prior biological sample from the subject indicates a response to the cancer therapy with an ability to inhibit function and/or activity of PD-1, PD-L1 and/or PD-L2.

32. The method of any one of claim 1 to 3, 7 to 9, 13 to 18, 22 to 24, or 31, wherein a lack of upregulation of one or more genes associated with type I IFN signaling pathway compared to a patient that is known to be sensitive to anti-PD-1 therapy indicates a response to the cancer therapy with an ability to inhibit function and/or activity of PD-1, PD-L1 and/or PD-L2.

33. The method of any one of claim 1 to 3, 7 to 9, 13 to 18, 22 to 24, 31 or 32, wherein a lack of upregulation of one or more genes associated with type I IFN signaling pathway compared to a standard indicates a response to the cancer therapy with an ability to inhibit function and/or activity of PD-1, PD-L1 and/or PD-L2.

34. The method of any one of claims 1 to 33, wherein the biological sample is a fresh tissue sample, frozen tumor tissue specimen, cultured cells, circulating tumor cells, or a formalin-fixed paraffin-embedded tumor tissue specimen.

35. The method of any one of claims 1 to 34, wherein the biological sample is a biopsy sample.

36. The method of claim 35, wherein the biopsy sample is selected from endoscopic biopsy, bone marrow biopsy, endoscopic biopsy (e.g., cystoscopy, bronchoscopy and colonoscopy), needle biopsy (e.g., fine-needle aspiration, core needle biopsy, vacuum-assisted biopsy, X-ray-assisted biopsy, computerized tomography (CT)-assisted biopsy, magnetic resonance imaging (MRI)-assisted biopsy and ultrasound-assisted biopsy), skin biopsy (e.g., shave biopsy, punch biopsy, and incisional biopsy) and surgical biopsy.

37. The method of any one of claims 1 to 34, wherein the biological sample comprises a body fluid selected from blood, plasma, serum, lacrimal fluid, tears, bone marrow, blood, blood cells, ascites, tissue or fine needle biopsy sample, cell-containing body fluid, free floating nucleic acids, sputum, saliva, urine, cerebrospinal fluid, peritoneal fluid, pleural fluid, feces, lymph, gynecological fluid, skin swab, vaginal swab, oral swab, nasal swab, washing or lavage such as a ductal lavage or broncheoalveolar lavage, aspirate, scraping, bone marrow specimen, tissue biopsy specimen, surgical specimen, feces, other body fluids, secretions, and/or excretions, and/or cells therefrom.

38. The method of any one of claims 1 to 37, wherein the biological sample is obtained by a technique selected from scrapes, swabs, and biopsy.

39. The method of claim 38, wherein the biological sample is obtained by use of brushes, (cotton) swabs, spatula, rinse/wash fluids, punch biopsy devices, puncture of cavities with needles or surgical instrumentation.

40. The method of any one of claims 34 to 39, wherein the biological sample comprises at least one tumor cell.

41. The method of claim 40, wherein the tumor is selected from Hodgkin's and non-Hodgkin's lymphoma, B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; or chronic myeloblastic leukemia, basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (e.g., that associated with brain tumors), Meigs' syndrome cancer; renal carcinoma; colorectal cancer; and adrenal cancer.

42. The method of any one of claims 1 to 41, wherein the evaluating is performed by DNA sequencing, RNA sequencing, immunohistochemical staining, western blotting, in cell western, immunofluorescent staining, ELISA, and fluorescent activating cell sorting (FACS) or a combination thereof.

43. The method of any one of claims 1 to 42, wherein the evaluating is performed by contacting the sample with an agent that specifically binds to one or more proteins encoded by one or more genes associated with a gene ontology (GO) pathway selected from:
(i) positive regulation of cell cycle process, regulation of G1/S transition, regulation of cell division, regulation of cell proliferation, positive regulation of IκB kinase/NFκB signaling, type I IFN signaling pathway, cellular response to IFNγ, positive regulation of IFNα production, positive regulation of defense response, positive regulation of IFNβ production, regulation of inflammatory response, regulation of innate immune response, negative regulation of antigen processing/presentation, and antigen processing/presentation of endogenous peptides via MHC class I; and/or
(ii) phospholipid efflux, negative regulation of fibrinolysis, chylomicron assembly, plasma membrane repair, SRP-dependent co-translational protein targeting to membrane, ribosomal small subunit assembly, phospholipid efflux, regulation of translation, mitochondrial respiratory chain complex I, mitochondrial translational elongation, DNA-dependent DNA replication, and ATP biosynthetic process.

44. The method of claim 43, wherein the evaluating is performed by contacting the sample with an agent that specifically binds to one or more proteins encoded by one or more genes associated with a gene ontology (GO) pathway selected from (a) cellular response to IFNγ, (b) negative regulation of antigen processing/presentation, (c) type I IFN signaling pathway, (d) positive regulation of IκB kinase/NFκB signaling, and antigen processing, and (e) presentation of endogenous peptides via MHC class I.

45. The method of claim 43 or claim 44, wherein the evaluating is performed by contacting the sample with an agent that specifically binds to one or more proteins encoded by one or more genes associated with cellular response to IFNγ.

46. The method of any one of claims 43 to 45, wherein the evaluating is performed by contacting the sample with an agent that specifically binds to one or more proteins encoded by one or more genes associated with type I IFN signaling pathway.

47. The method of any one of claims 1 to 42, wherein the evaluating is performed by contacting the sample with an agent that specifically binds to one or more of nucleic acids of one or more genes associated with a gene ontology (GO) pathway selected from:
(i) positive regulation of cell cycle process, regulation of G1/S transition, regulation of cell division, regulation of cell proliferation, positive regulation of IκB kinase/NFκB signaling, type I IFN signaling pathway, cellular response to IFNγ, positive regulation of IFNα production, positive regulation of defense response, positive regulation of IFNβ production, regulation of inflammatory response, regulation of innate immune response, negative regulation of antigen processing/presentation, and antigen processing/presentation of endogenous peptides via MHC class I; and/or
(ii) phospholipid efflux, negative regulation of fibrinolysis, chylomicron assembly, plasma membrane repair, SRP-dependent co-translational protein targeting to membrane, ribosomal small subunit assembly, phospholipid efflux, regulation of translation, mitochondrial respiratory chain complex I, mitochondrial translational elongation, DNA-dependent DNA replication, and ATP biosynthetic process.

48. The method of claim 47, wherein the evaluating is performed by contacting the sample with an agent that specifically binds to one or more nucleic acid of one or more genes associated with a gene ontology (GO) pathway selected from (a) cellular response to IFNγ, (b) negative regulation of antigen processing/presentation, (c) type I IFN signaling pathway, (d) positive regulation of IκB kinase/NFκB signaling, and antigen processing, and (e) presentation of endogenous peptides via MHC class I.

49. The method of claim 47 or claim 48, wherein the evaluating is performed by contacting the sample with an agent that specifically binds to one or more nucleic acid of one or more genes associated with cellular response to IFNγ.

50. The method of any one of claims 47 to 49, wherein the evaluating is performed by contacting the sample with an agent that specifically binds to one or more nucleic acid of one or more genes associated with type I IFN signaling pathway.

51. The method of any one of claims 47 to 50, wherein the agent that specifically binds to one or more of the nucleic acids is a nucleic acid primer or probe.

52. The method of any one of claims 1 to 51, wherein the evaluating informs classifying the patient into a high or low risk group.

53. The method of claim 52, wherein the high risk classification comprises a high level of tumor cells having resistance to the cancer therapy with an ability to inhibit function and/or activity of PD-1, PD-L1 and/or PD-L2.

54. The method of claim 52, wherein the low risk classification comprises a low level of tumor cells having resistance to the cancer therapy with an ability to inhibit function and/or activity of PD-1, PD-L1 and/or PD-L2.

55. The method of any one of claims 52 to 54, wherein the low risk or high risk classification is indicative of withholding of a neoadjuvant therapy.

56. The method of any one of claims 52 to 54, wherein the low risk or high risk classification is indicative of withholding of an adjuvant therapy.

57. The method of any one of claims 52 to 54, wherein the evaluating is predictive of a positive response to and/or benefit from the cancer treatment.

58. The method of any one of claims 52 to 54, wherein the evaluating is predictive of a negative or neutral response to and/or benefit from the cancer treatment.

59. The method of any one of claims 52 to 54, wherein the evaluating is predictive of a positive response to and/or benefit from neoadjuvant chemotherapy or a non-responsiveness to and/or lack of benefit from neoadjuvant chemotherapy.

60. The method of any one of claims 52 to 54, wherein the evaluating is predictive of a positive response to and/or benefit from adjuvant chemotherapy or a non-responsiveness to and/or lack of benefit from adjuvant chemotherapy.

61. The method of any one of claims 52 to 54, wherein the evaluating is predictive of a negative or neutral response to and/or benefit from neoadjuvant chemotherapy or a non-responsiveness to and/or lack of benefit from neoadjuvant chemotherapy.

62. The method of any one of claims 52 to 54, wherein the evaluating is predictive of a negative or neutral response to and/or benefit from adjuvant chemotherapy or a non-responsiveness to and/or lack of benefit from adjuvant chemotherapy.

63. The method of any one of claims 52 to 54, wherein the evaluating informs administration or withholding of the cancer treatment.

64. The method of any one of claims 52 to 54, wherein the evaluating informs administration of neoadjuvant therapy.

65. The method of any one of claims 52 to 54, wherein the evaluating informs administration of adjuvant therapy.

66. The method of any one of claims 52 to 54, wherein the evaluating informs withholding of neoadjuvant therapy.

67. The method of any one of claims 52 to 54, wherein the evaluating informs withholding of adjuvant therapy.

68. The method of any one of claims 55 to 67, wherein the neoadjuvant therapy and/or the adjuvant therapy is selected from a chemotherapeutic agent, a cytotoxic agent, a checkpoint inhibitor, an antimetabolite chemotherapeutic (e.g., 5-fluorouracil, methotrexate, capecitabine, azacitidine), a topoisomerase inhibitor (e.g., irinotecan, topotecan, etoposide, doxorubicin, etc.).

69. The method of any one of claims 55 to 68, wherein the neoadjuvant therapy and/or the adjuvant therapy is selected from a protein translation inhibitor (e.g., a modulator of assembly and/or function of ribosomal complex, a modulator of expression and/or function of tRNA, a modulator of synthesis and/or uptake of amino acids, a modulator of post-translational modification (e.g., decoration of the translated protein with carbohydrates), a modulator of protein degradation, and a modulator of protein transport (e.g., post-translational peptide processing, signal peptide recognition and cleavage, transport through the ER/Golgi network, etc.), etc.) or topoisomerase inhibitors.

70. The method of claim 69, wherein the neoadjuvant therapy and/or the adjuvant therapy is selected from a protein translation inhibitor (e.g., silvestrol and omacetaxine) ribosome biogenesis inhibitors (e.g., diazaborine, lamotrigine and ribozinoindoles), inhibitors of rRNA and/or tRNA synthesis (e.g., quarfloxin (CX-3543) and CX-5461), an inhibitor of synthesis of amino acids (e.g., GLUD1 inhibitor R162, BCAT1 inhibitor gabapentin, glutaminase inhibitor bis-2-(5-phenylacetamido-1,2,4-thiadiazol-2-yl)ethyl sulfide (BPTES), PAGDH inhibitor NCT-503), an inhibitor of uptake of amino acids (e.g., SLC7A11 inhibitors sulfasalazine, erastin or sorafenib), a modulator of post-translational modification (e.g., glycosylation inhibitor tunicamycin, ppGalNAc-T3), a modulator of protein degradation, and a modulator of protein transport (e.g., cyclosporin A, fendiline, parbendazole, paroxetine, parthenolide, quinacrine, sertraline, spiperone, thimerosal, astemizole, perhexiline, HUN-7293, CAM741, CK147, and cotransin).

71. A transgenic non-human animal comprising one or more tumor cells, wherein the tumor cells have:
(a) upregulation of one or more genes associated with a gene ontology (GO) pathway selected from: positive regulation of cell cycle process, regulation of G1/S transition, regulation of cell division, regulation of cell proliferation, positive regulation of IκB kinase/NFκB signaling, type I IFN signaling pathway, cellular response to IFNγ, positive regulation of IFNα production, positive regulation of defense response, positive regulation of IFNβ production, regulation of inflammatory response, regulation of innate immune response, negative regulation of antigen processing/presentation, and antigen processing/presentation of endogenous peptides via MHC class I; and/or
(b) downregulation of one or more genes associated with a gene ontology (GO) pathway selected from: phospholipid efflux, negative regulation of fibrinolysis, chylomicron assembly, plasma membrane repair, SRP-dependent co-translational protein targeting to membrane, ribosomal small subunit assembly, phospholipid efflux, regulation of translation, mitochondrial respiratory chain complex I, mitochondrial translational elongation, DNA-dependent DNA replication, and ATP biosynthetic process.

72. The transgenic non-human animal of claim 71, wherein the tumor cells are resistant to a cancer therapy with an ability to inhibit function and/or activity of PD-1, PD-L1 and/or PD-L2.

73. The transgenic non-human animal of claim 72, wherein the cancer therapy with an ability to inhibit function and/or activity of PD-1, PD-L1 and/or PD-L2 is an antibody.

74. The transgenic non-human animal of any one of claims 71 to 73, wherein the antibody is selected from nivolumab (OPDIVO), pembrolizumab (KEYTRUDA), pidilizumab (CT-011, CURE TECH), MK-3475 (MERCK), BMS 936559, MPDL3280A (ROCHE), Cemiplimab (LIBTAYO), Atezolizumab (TECENTRIQ), Avelumab (BAVENCIO), and Durvalumab (imfinzi).

75. The transgenic non-human animal of any one of claims 71 to 74, wherein the one or more tumor cells have:
(a) upregulation of one or more genes associated with a gene ontology (GO) pathway selected from: positive regulation of cell cycle process, regulation of G1/S transition, regulation of cell division, regulation of cell proliferation, positive regulation of IκB kinase/NFκB signaling, type I IFN signaling pathway, cellular response to IFNγ, positive regulation of IFNα production, positive regulation of defense response, positive regulation of IFNβ production, regulation of inflammatory response, regulation of innate immune response, negative regulation of antigen processing/presentation, and antigen processing/presentation of endogenous peptides via MHC class I; and/or
(b) downregulation of one or more genes associated with a gene ontology (GO) pathway selected from: phospholipid efflux, negative regulation of fibrinolysis, chylomicron assembly, plasma membrane repair, SRP-dependent co-translational protein targeting to membrane, ribosomal small subunit assembly, phospholipid efflux, regulation of translation, mitochondrial respiratory chain complex I, mitochondrial translational elongation, DNA-dependent DNA replication, and ATP biosynthetic process.

76. The transgenic non-human animal of claim 75, wherein the one or more tumor cells have an upregulation of one or more genes associated with cellular response to IFNγ.

77. The transgenic non-human animal of claim 75 or claim 76, wherein the one or more tumor cells have an upregulation of one or more genes associated with type I IFN signaling pathway.

78. The transgenic non-human animal of any one of claims 71 to 77, wherein the transgenic non-human animal is a rodent.

79. The transgenic non-human animal of claim 78, wherein the rodent is a mouse.

80. The transgenic non-human animal of claim 79, wherein the mouse belongs to BALB/c or C57BL/6 strain.

81. A method of making a transgenic non-human animal comprising one or more cancer cells that are nonresponsive, resistant, or recalcitrance to a cancer therapy, wherein the cancer therapy has an ability to inhibit function and/or activity of PD-1, PD-L1 and/or PD-L2, the method comprising:
(a) injecting one or more parental cancer cells that are responsive to the cancer therapy in a non-human animal;
(b) administering the cancer therapy to the non-human animal;
(c) isolating cancer cells that survived the cancer therapy;
(d) injecting cancer cells that survived the cancer therapy in a different non-human animal of the same species; and
(e) repeating steps (b) to (d) two to ten more times.

82. The method of claim 81, wherein steps (b) to (d) are repeated at least once more.

83. The method of claim 81, wherein steps (b) to (d) are repeated at least twice more.

84. The method of claim 81, wherein steps (b) to (d) are repeated at least three times more.

85. The method of claim 81, wherein steps (b) to (d) are repeated less than five times.

86. The method of any one of claims 81 to 85, wherein the transgenic non-human animal is a rodent.

87. The method of claim 86, wherein the rodent is a mouse.

88. The method of claim 87, wherein the mouse belongs to BALB/c or C57BL/6 strain.

89. The method of any one of claims 81 to 88, wherein the cancer therapy that has the ability inhibit function and/or activity of PD-1, PD-L1 and/or PD-L2 is an antibody.

90. The method of claim 89, wherein the antibody is selected from nivolumab (OPDIVO), pembrolizumab (KEYTRUDA), pidilizumab (CT-011, CURE TECH), MK-3475 (MERCK), BMS 936559, MPDL3280A (ROCHE), Cemiplimab (LIBTAYO), Atezolizumab (TECENTRIQ), Avelumab (BAVENCIO), and Durvalumab (imfinzi).

91. The method of any one of claims 81 to 90, wherein the cancer therapy is capable of inhibiting the growth of tumors when administered to a transgenic non-human animal transgenic non-human animal harboring a parental cancer cell tumor compared to an untreated transgenic non-human animal harboring a parental cancer cell tumors.

92. The method of claims 81 to 91, wherein the tumor cells that survived the cancer therapy have:
(a) upregulation of one or more genes associated with a gene ontology (GO) pathway selected from: positive regulation of cell cycle process, regulation of G1/S transition, regulation of cell division, regulation of cell proliferation, positive regulation of IκB kinase/NFκB signaling, type I IFN signaling pathway, cellular response to IFNγ, positive regulation of IFNα production, positive regulation of defense response, positive regulation of IFNβ production, regulation of inflammatory response, regulation of innate immune response, negative regulation of antigen processing/presentation, and antigen processing/presentation of endogenous peptides via MHC class I; and/or
(b) downregulation of one or more genes associated with a gene ontology (GO) pathway selected from: phospholipid efflux, negative regulation of fibrinolysis, chylomicron assembly, plasma membrane repair, SRP-dependent co-translational protein targeting to membrane, ribosomal small subunit assembly, phospholipid efflux, regulation of translation, mitochondrial respiratory chain complex I, mitochondrial translational elongation, DNA-dependent DNA replication, and ATP biosynthetic process.

93. The method of claim 92, wherein the one or more tumor cells have an upregulation of one or more genes associated with cellular response to IFNγ and/or type I IFN signaling pathway.

94. A transgenic animal made according to the method of any one of claims 81 to 93.

95. A method for testing an anti-cancer drug candidate, the method comprising:
(a) providing a transgenic non-human animal of any one of claims 71 to 80 or a transgenic non-human animal made according to the method of any one of claims 81 to 94;
(b) administering the anti-cancer drug candidate to the transgenic non-human animal, and
(c) evaluating whether the anti-cancer drug candidate is effective in slowing or inhibiting cancer growth in the transgenic non-human animal.

96. A method for making a pharmaceutical composition for treating cancer, the method comprising:
(a) providing a transgenic non-human animal of any one of claims 71 to 80 or a transgenic non-human animal made according to the method of any one of claims 81 to 94;
(b) administering the anti-cancer drug candidate to the transgenic non-human animal, and
(c) selecting an anti-cancer drug that is effective in slowing or inhibiting cancer growth in the transgenic non-human animal; and
(d) formulating the anti-cancer drug or candidate for administration to a human patient.

97. The method of claim 95 or claim 96, wherein the anti-cancer drug candidate is selected from a chemotherapeutic agent, a cytotoxic agent, and a checkpoint inhibitor.

98. A method of determining a cancer treatment for a patient, the method comprising:
(a) obtaining a biological sample from a subject;
(b) evaluating the biological sample for the expression of:
(i) a gene selected from CD274, B2M, STAT1, STAT2, TRIM7, IRF1, TAP1, TAP2, CASP1, IRF, LTBR, PVR, GASTA3, LRG1, SPRY2, ARG1, TRIM8, TRIM2, MAPK81P1, TRIM6, and KRT1; and/or
(ii) a gene selected from RPL41, RPS15, RPS8, TRIM7 and LRG1; and
(c) selecting the cancer therapy based on the evaluation of step (b), wherein cancer therapy is selected from:
(i) an agent with an ability to inhibit function and/or activity of PD-1, PD-L1 and/or PD-L2;
(ii) an antimetabolite chemotherapeutic (e.g., 5-fluorouracil, methotrexate, capecitabine, azacitidine, 6-diazo-5-oxo-L-norleucine (DON), azaserine and acivicin), a topoisomerase inhibitor (e.g., irinotecan, topotecan, etoposide, doxorubicin, etc.); and
(iii) a protein translation inhibitor (e.g., silvestrol and omacetaxine) ribosome biogenesis inhibitors (e.g., diazaborine, lamotrigine and ribozinoindoles), inhibitors of rRNA and/or tRNA synthesis (e.g., quarfloxin (CX-3543) and CX-5461), an inhibitor of synthesis of amino acids (e.g., GLUD1 inhibitor R162, BCAT1 inhibitor gabapentin, glutaminase inhibitor bis-2-(5-phenylacetamido-1,2,4-thiadiazol-2-yl)ethyl sulfide (BPTES), PAGDH inhibitor NCT-503), an inhibitor of uptake of amino acids (e.g., SLC7A11 inhibitors sulfasalazine, erastin or sorafenib), a modulator of post-translational modification (e.g., glycosylation inhibitor tunicamycin, ppGalNAc-T3), a modulator of protein degradation, and a modulator of protein transport (e.g., cyclosporin A, fendiline, parbendazole, paroxetine, parthenolide, quinacrine, sertraline, spiperone, thimerosal, astemizole, perhexiline, HUN-7293, CAM741, CK147, and cotransin, etc.) or a topoisomerase inhibitor.

99. A method for selecting a patient for a cancer treatment, the method comprising:
(a) obtaining a biological sample from a subject;
(b) evaluating the biological sample for the expression of:
(i) a gene selected from CD274, B2M, STAT1, STAT2, TRIM7, IRF1, TAP1, TAP2, CASP1, IRF, LTBR, PVR, GASTA3, LRG1, SPRY2, ARG1, TRIM8, TRIM2, MAPK81P1, TRIM6, and KRT1; and/or
(ii) a gene selected from RPL41, RPS15, RPS8, TRIM7 and LRG1; and
and
(c) selecting the cancer therapy based on the evaluation of step (b), wherein cancer therapy is selected from:
(i) an agent with an ability to inhibit function and/or activity of PD-1, PD-L1 and/or PD-L2;
(ii) an antimetabolite chemotherapeutic (e.g., 5-fluorouracil, methotrexate, capecitabine, azacitidine, 6-diazo-5-oxo-L-norleucine (DON), azaserine and acivicin), a topoisomerase inhibitor (e.g., irinotecan, topotecan, etoposide, doxorubicin, etc.); and
(iii) a protein translation inhibitor (e.g., silvestrol and omacetaxine) ribosome biogenesis inhibitors (e.g., diazaborine, lamotrigine and ribozinoindoles), inhibitors of rRNA and/or tRNA synthesis (e.g., quarfloxin (CX-3543) and CX-5461), an inhibitor of synthesis of amino acids (e.g., GLUD1 inhibitor R162, BCAT1 inhibitor gabapentin, glutaminase inhibitor bis-2-(5-phenylacetamido-1,2,4-thiadiazol-2-yl)ethyl sulfide (BPTES), PAGDH inhibitor NCT-503), an inhibitor of uptake of amino acids (e.g., SLC7A11 inhibitors sulfasalazine, erastin or sorafenib), a modulator of post-translational modification (e.g., glycosylation inhibitor tunicamycin, ppGalNAc-T3), a modulator of protein degradation, and a modulator of protein transport (e.g., cyclosporin A, fendiline, parbendazole, paroxetine, parthenolide, quinacrine, sertraline, spiperone, thimerosal, astemizole, perhexiline, HUN-7293, CAM741, CK147, and cotransin, etc.) or a topoisomerase inhibitor.

100. A method of treating cancer, the method comprising:
(a) obtaining a biological sample from a subject;
(b) evaluating the biological sample for the expression of:
(i) a gene selected from CD274, B2M, STAT1, STAT2, TRIM7, IRF1, TAP1, TAP2, CASP1, IRF, LTBR, PVR, GASTA3, LRG1, SPRY2, ARG1, TRIM8, TRIM2, MAPK81P1, TRIM6, and KRT1; and/or
(ii) a gene selected from RPL41, RPS15, RPS8, TRIM7 and LRG1; and
(c) selecting the cancer therapy selected from:
(i) an agent with an ability to inhibit function and/or activity of PD-1, PD-L1 and/or PD-L2;
(ii) an antimetabolite chemotherapeutic (e.g., 5-fluorouracil, methotrexate, capecitabine, azacitidine, 6-diazo-5-oxo-L-norleucine (DON), azaserine and acivicin), a topoisomerase inhibitor (e.g., irinotecan, topotecan, etoposide, doxorubicin, etc.); and
(iii) a protein translation inhibitor (e.g., silvestrol and omacetaxine) ribosome biogenesis inhibitors (e.g., diazaborine, lamotrigine and ribozinoindoles), inhibitors of rRNA and/or tRNA synthesis (e.g., quarfloxin (CX-3543) and CX-5461), an inhibitor of synthesis of amino acids (e.g., GLUD1 inhibitor R162, BCAT1 inhibitor gabapentin, glutaminase inhibitor bis-2-(5-phenylacetamido-1,2,4-thiadiazol-2-yl)ethyl sulfide (BPTES), PAGDH inhibitor NCT-503), an inhibitor of uptake of amino acids (e.g., SLC7A11 inhibitors sulfasalazine, erastin or sorafenib), a modulator of post-translational modification (e.g., glycosylation inhibitor tunicamycin, ppGalNAc-T3), a modulator of protein degradation, and a modulator of protein transport (e.g., cyclosporin A, fendiline, parbendazole, paroxetine, parthenolide, quinacrine, sertraline, spiperone, thimerosal, astemizole, perhexiline, HUN-7293, CAM741, CK147, and cotransin, etc.) or a topoisomerase inhibitor; and
(d) administering the cancer therapy with an ability to inhibit function and/or activity of PD-1, PD-L1 and/or PD-L2; and optionally administering the therapy selected in step (c)(ii) and (c)(iii).

101. The method of any one of claims 98 to 100, wherein the cancer therapy with an ability to inhibit function and/or activity of PD-1, PD-L1 and/or PD-L2 is selected when the biological sample comprises at least one tumor cell, and
a gene selected from CD274, B2M, STAT1, STAT2, TRIM7, IRF1, TAP1, TAP2, CASP1, IRF, LTBR, PVR, GASTA3, LRG1, SPRY2, ARG1, TRIM8, TRIM2, MAPK8IP1, TRIM6, and KRT1 is not upregulated in the at least one tumor cell compared to a healthy tissue, a prior biological sample obtained from the subject, or another biological sample from patient that is known to be sensitive to anti-PD-1 therapy; and/or
a gene selected from RPL41, RPS15, RPS8, TRIM7 and LRG1 is not downregulated in the at least one tumor cell compared to a healthy tissue, a prior biological sample obtained from the subject, or another biological sample from patient that is known to be sensitive to anti-PD-1 therapy.

102. The method of any one of claims 98 to 100, wherein, when the biological sample comprises at least one tumor cell, and
a gene selected from CD274, B2M, STAT1, STAT2, TRIM7, IRF1, TAP1, TAP2, CASP1, IRF, LTBR, PVR, GASTA3, LRG1, SPRY2, ARG1, TRIM8, TRIM2, MAPK8IP1, TRIM6, and KRT1 is upregulated in the at least one tumor cell compared to a healthy tissue, a prior biological sample obtained from the subject, or another biological sample from patient that is known to be sensitive to anti-PD-1 therapy; and/or
a gene selected from RPL41, RPS15, RPS8, TRIM7 and LRG1 is downregulated in the at least one tumor cell compared to a healthy tissue, a prior biological sample obtained from the subject, or another biological sample from patient that is known to be sensitive to anti-PD-1 therapy, the cancer therapy is selected from:
an antimetabolite chemotherapeutic, a topoisomerase inhibitor, a protein translation inhibitor, a ribosome biogenesis inhibitor, an inhibitor of rRNA and/or tRNA synthesis, an inhibitor of synthesis of amino acids, an inhibitor of uptake of amino acids, a modulator of post-translational modification, a modulator of protein degradation, a modulator of protein transport, a topoisomerase inhibitor.

103. The method of any one of claims 98 to 102, wherein an upregulation of one or more genes listed in (b)(i) compared to a healthy tissue indicates a lack of response, resistance or recalcitrance to the cancer therapy with an ability to inhibit function and/or activity of PD-1, PD-L1 and/or PD-L2.

104. The method of any one of claims 98 to 103, wherein a downregulation of one or more genes listed in (b)(ii) compared to a healthy tissue indicates a lack of response, resistance or recalcitrance to the cancer therapy with an ability to inhibit function and/or activity of PD-1, PD-L1 and/or PD-L2.

105. The method of any one of claims 98 to 104, wherein an upregulation of one or more genes listed in (b)(i) compared to another biological sample from patient that is known to be sensitive to anti-PD-1 therapy indicates a lack of response, resistance or recalcitrance to the cancer therapy with an ability to inhibit function and/or activity of PD-1, PD-L1 and/or PD-L2.

106. The method of any one of claims 98 to 104, wherein a downregulation of one or more genes listed in (b)(ii) compared to another biological sample from patient that is known to be sensitive to anti-PD-1 therapy indicates a lack of response, resistance or recalcitrance to the cancer therapy with an ability to inhibit function and/or activity of PD-1, PD-L1 and/or PD-L2.

107. The method of any one of claims 98 to 106, wherein an upregulation of one or more genes listed in (b)(i) compared to a prior biological sample obtained from the subject indicates a development of a lack of response, resistance or recalcitrance to the cancer therapy with an ability to inhibit function and/or activity of PD-1, PD-L1 and/or PD-L2.

108. The method of any one of claims 98 to 107, wherein a downregulation of one or more genes listed in (b)(ii) compared to a prior biological sample obtained from the subject indicates a development of a lack of response, resistance or recalcitrance to the cancer therapy with an ability to inhibit function and/or activity of PD-1, PD-L1 and/or PD-L2.

109. The method of any one of claims 98 to 108, wherein a lack of upregulation of one or more genes listed in (b)(i) compared to a healthy tissue indicates a response to the cancer therapy with an ability to inhibit function and/or activity of PD-1, PD-L1 and/or PD-L2.

110. The method of any one of claims 98 to 109, wherein a lack of downregulation of one or more genes listed in (b)(ii) compared to a healthy tissue indicates a response to the cancer therapy with an ability to inhibit function and/or activity of PD-1, PD-L1 and/or PD-L2.

111. The method of any one of claims 98 to 110, wherein a lack of upregulation of one or more genes listed in (b)(i) compared to another biological sample from patient that is known to be sensitive to anti-PD-1 therapy indicates a response to the cancer therapy with an ability to inhibit function and/or activity of PD-1, PD-L1 and/or PD-L2.

112. The method of any one of claims 98 to 111, wherein a lack of downregulation of one or more genes listed in (b)(ii) compared to another biological sample from patient that is known to be sensitive to anti-PD-1 therapy indicates a response to the cancer therapy with an ability to inhibit function and/or activity of PD-1, PD-L1 and/or PD-L2.

113. The method of any one of claims 98 to 112, wherein a lack of upregulation of one or more genes listed in (b)(i) compared to a prior biological sample obtained from the subject indicates a development of lack of a response to the cancer therapy with an ability to inhibit function and/or activity of PD-1, PD-L1 and/or PD-L2.

114. The method of any one of claims 98 to 113, wherein a lack of downregulation of one or more genes listed in (b)(ii) compared to a prior biological sample obtained from the subject indicates a development of lack of a response to the cancer therapy with an ability to inhibit function and/or activity of PD-1, PD-L1 and/or PD-L2.

115. The method of any one of claims 98 to 114, wherein the biological sample is a fresh tissue sample, frozen tumor tissue specimen, cultured cells, circulating tumor cells, or a formalin-fixed paraffin-embedded tumor tissue specimen.

116. The method of any one of claims 98 to 115, wherein the biological sample is a biopsy sample, optionally wherein the biopsy sample is selected from endoscopic biopsy, bone marrow biopsy, endoscopic biopsy (e.g., cystoscopy, bronchoscopy and colonoscopy), needle biopsy (e.g., fine-needle aspiration, core needle biopsy, vacuum-assisted biopsy, X-ray-assisted biopsy, computerized tomography (CT)-assisted biopsy, magnetic resonance imaging (MRI)-assisted biopsy and ultrasound-assisted biopsy), skin biopsy (e.g., shave biopsy, punch biopsy, and incisional biopsy) and surgical biopsy.

117. The method of any one of claims 98 to 116, wherein the biological sample comprises a body fluid selected from blood, plasma, serum, lacrimal fluid, tears, bone marrow, blood, blood cells, ascites, tissue or fine needle biopsy sample, cell-containing body fluid, free floating nucleic acids, sputum, saliva, urine, cerebrospinal fluid, peritoneal fluid, pleural fluid, feces, lymph, gynecological fluid, skin swab, vaginal swab, oral swab, nasal swab, washing or lavage such as a ductal lavage or broncheoalveolar lavage, aspirate, scraping, bone marrow specimen, tissue biopsy specimen, surgical specimen, feces, other body fluids, secretions, and/or excretions, and/or cells therefrom.

118. The method of any one of claims 98 to 117, wherein the biological sample is obtained by a technique selected from scrapes, swabs, and biopsy, optionally wherein the biological sample is obtained by use of brushes, (cotton) swabs, spatula, rinse/wash fluids, punch biopsy devices, puncture of cavities with needles or surgical instrumentation.

119. The method of any one of claims 115 to 118, wherein the biological sample comprises at least one tumor cell.

120. The method of claim 101, 102 or 119, wherein the tumor is selected from Hodgkin's and non-Hodgkin's lymphoma, B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; or chronic myeloblastic leukemia, basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (e.g., that associated with brain tumors), Meigs' syndrome cancer; renal carcinoma; colorectal cancer; and adrenal cancer.

121. The method of any one of claims 98 to 120, wherein the evaluating is performed by DNA sequencing, RNA sequencing, immunohistochemical staining, western blotting, in cell western, immunofluorescent staining, ELISA, and fluorescent activating cell sorting (FACS) or a combination thereof.

122. The method of any one of claims 98 to 121, wherein the evaluating is performed by contacting the sample with an agent that specifically binds to one or more proteins encoded by one or more genes listed in (b)(i) and/or (b)(ii), optionally wherein the agent that specifically binds to one or proteins comprises an antibody, antibody-like molecule or binding a fragment thereof.

123. The method of any one of claims 98 to 122, wherein the evaluating is performed by contacting the sample with an agent that specifically binds to one or more of nucleic acids of one or more genes associated with a gene listed in (b)(i) and/or (b)(ii).

124. The method of claim 123, wherein the agent that specifically binds to one or more of the nucleic acids is a nucleic acid primer or probe.

125. A method of determining a cancer treatment for a patient, the method comprising:
(a) obtaining a biological sample from a subject;
(b) evaluating the biological sample for the activation of a pathway selected from Mapk8ip1, Trim7, Elk1, Lrg1, Arg1, Rap1, and Ras; and
(c) selecting the cancer therapy based on the evaluation of step (b), wherein cancer therapy is selected from:
(i) an agent with an ability to inhibit function and/or activity of PD-1, PD-L1 and/or PD-L2;
(ii) an antimetabolite chemotherapeutic (e.g., 5-fluorouracil, methotrexate, capecitabine, azacitidine, 6-diazo-5-oxo-L-norleucine (DON), azaserine and acivicin), a topoisomerase inhibitor (e.g., irinotecan, topotecan, etoposide, doxorubicin, etc.); and
(iii) a protein translation inhibitor (e.g., silvestrol and omacetaxine) ribosome biogenesis inhibitors (e.g., diazaborine, lamotrigine and ribozinoindoles), inhibitors of rRNA and/or tRNA synthesis (e.g., quarfloxin (CX-3543) and CX-5461), an inhibitor of synthesis of amino acids (e.g., GLUD1 inhibitor R162, BCAT1 inhibitor gabapentin, glutaminase inhibitor bis-2-(5-phenylacetamido-1,2,4-thiadiazol-2-yl)ethyl sulfide (BPTES), PAGDH inhibitor NCT-503), an inhibitor of uptake of amino acids (e.g., SLC7A11 inhibitors sulfasalazine, erastin or sorafenib), a modulator of post-translational modification (e.g., glycosylation inhibitor tunicamycin, ppGalNAc-T3), a modulator of protein degradation, and a modulator of protein transport (e.g., cyclosporin A, fendiline, parbendazole, paroxetine, parthenolide, quinacrine, sertraline, spiperone, thimerosal, astemizole, perhexiline, HUN-7293, CAM741, CK147, and cotransin, etc.) or a topoisomerase inhibitor.

126. A method for selecting a patient for a cancer treatment, the method comprising:
(a) obtaining a biological sample from a subject;
(b) evaluating the biological sample for the activation of a pathway selected from Mapk8ip1, Trim7, Elk1, Lrg1, Arg1, Rap1, and Ras; and
(c) selecting the cancer therapy based on the evaluation of step (b), wherein cancer therapy is selected from:
(i) an agent with an ability to inhibit function and/or activity of PD-1, PD-L1 and/or PD-L2;
(ii) an antimetabolite chemotherapeutic (e.g., 5-fluorouracil, methotrexate, capecitabine, azacitidine, 6-diazo-5-oxo-L-norleucine (DON), azaserine and acivicin), a topoisomerase inhibitor (e.g., irinotecan, topotecan, etoposide, doxorubicin, etc.); and
(iii) a protein translation inhibitor (e.g., silvestrol and omacetaxine) ribosome biogenesis inhibitors (e.g., diazaborine, lamotrigine and ribozinoindoles), inhibitors of rRNA and/or tRNA synthesis (e.g., quarfloxin (CX-3543) and CX-5461), an inhibitor of synthesis of amino acids (e.g., GLUD1 inhibitor R162, BCAT1 inhibitor gabapentin, glutaminase inhibitor bis-2-(5-phenylacetamido-1,2,4-thiadiazol-2-yl)ethyl sulfide (BPTES), PAGDH inhibitor NCT-503), an inhibitor of uptake of amino acids (e.g., SLC7A11 inhibitors sulfasalazine, erastin or sorafenib), a modulator of post-translational modification (e.g., glycosylation inhibitor tunicamycin, ppGalNAc-T3), a modulator of protein degradation, and a modulator of protein transport (e.g., cyclosporin A, fendiline, parbendazole, paroxetine, parthenolide, quinacrine, sertraline, spiperone, thimerosal, astemizole, perhexiline, HUN-7293, CAM741, CK147, and cotransin, etc.) or a topoisomerase inhibitor.

127. A method of treating cancer, the method comprising:
(a) obtaining a biological sample from a subject;
(b) evaluating the biological sample for the activation of a pathway selected from Mapk8ip1, Trim7, Elk1, Lrg1, Arg1, Rap1, and Ras; and
(c) selecting the cancer therapy selected from:
(i) an agent with an ability to inhibit function and/or activity of PD-1, PD-L1 and/or PD-L2;
(ii) an antimetabolite chemotherapeutic (e.g., 5-fluorouracil, methotrexate, capecitabine, azacitidine, 6-diazo-5-oxo-L-norleucine (DON), azaserine and acivicin), a topoisomerase inhibitor (e.g., irinotecan, topotecan, etoposide, doxorubicin, etc.); and
(iii) a protein translation inhibitor (e.g., silvestrol and omacetaxine) ribosome biogenesis inhibitors (e.g., diazaborine, lamotrigine and ribozinoindoles), inhibitors of rRNA and/or tRNA synthesis (e.g., quarfloxin (CX-3543) and CX-5461), an inhibitor of synthesis of amino acids (e.g., GLUD1 inhibitor R162, BCAT1 inhibitor gabapentin, glutaminase inhibitor bis-2-(5-phenylacetamido-1,2,4-thiadiazol-2-yl)ethyl sulfide (BPTES), PAGDH inhibitor NCT-503), an inhibitor of uptake of amino acids (e.g., SLC7A11 inhibitors sulfasalazine, erastin or sorafenib), a modulator of post-translational modification (e.g., glycosylation inhibitor tunicamycin, ppGalNAc-T3), a modulator of protein degradation, and a modulator of protein transport (e.g., cyclosporin A, fendiline, parbendazole, paroxetine, parthenolide, quinacrine, sertraline, spiperone, thimerosal, astemizole, perhexiline, HUN-7293, CAM741, CK147, and cotransin, etc.) or a topoisomerase inhibitor; and
(d) administering the cancer therapy with an ability to inhibit function and/or activity of PD-1, PD-L1 and/or PD-L2; and optionally administering the therapy selected in step (c)(ii) and (c)(iii).

128. The method of any one of claims 125 to 127, wherein the cancer therapy with an ability to inhibit function and/or activity of PD-1, PD-L1 and/or PD-L2 is selected when the biological sample comprises at least one tumor cell, and
pathway selected from Mapk8ip1, Trim7, Elk1, Lrg1, Arg1, Rap1, and Ras is not upregulated in the at least one tumor cell compared to a healthy tissue, a prior biological sample obtained from the subject, or another biological sample from patient that is known to be sensitive to anti-PD-1 therapy.

129. The method of any one of claims 125 to 127, wherein, when the biological sample comprises at least one tumor cell, and
pathway selected from Mapk8ip1, Trim7, Elk1, Lrg1, Arg1, Rap1, and Ras is upregulated in the at least one tumor cell compared to a healthy tissue, a prior biological sample obtained from the subject, or another biological sample from patient that is known to be sensitive to anti-PD-1 therapy, the cancer therapy is selected from:
an antimetabolite chemotherapeutic, a topoisomerase inhibitor, a protein translation inhibitor, a ribosome biogenesis inhibitor, an inhibitor of rRNA and/or tRNA synthesis, an inhibitor of synthesis of amino acids, an inhibitor of uptake of amino acids, a modulator of post-translational modification, a modulator of protein degradation, a modulator of protein transport, a topoisomerase inhibitor.

130. The method of any one of claims 125 to 129, wherein an upregulation of pathway selected from Mapk8ip1, Trim7, Elk1, Lrg1, Arg1, Rap1, and Ras compared to a healthy tissue indicates a lack of response, resistance or recalcitrance to the cancer therapy with an ability to inhibit function and/or activity of PD-1, PD-L1 and/or PD-L2.

131. The method of any one of claims 125 to 130, wherein an upregulation of pathway selected from Mapk8ip1, Trim7, Elk1, Lrg1, Arg1, Rap1, and Ras compared to another biological sample from patient that is known to be sensitive to anti-PD-1 therapy indicates a lack of response, resistance or recalcitrance to the cancer therapy with an ability to inhibit function and/or activity of PD-1, PD-L1 and/or PD-L2.

132. The method of any one of claims 125 to 131, wherein an upregulation of pathway selected from Mapk8ip1, Trim7, Elk1, Lrg1, Arg1, Rap1, and Ras compared to a prior biological sample obtained from the subject indicates a development of a lack of response, resistance or recalcitrance to the cancer therapy with an ability to inhibit function and/or activity of PD-1, PD-L1 and/or PD-L2.

133. The method of any one of claims 125 to 132, wherein a lack of upregulation of pathway selected from Mapk8ip1, Trim7, Elk1, Lrg1, Arg1, Rap1, and Ras compared to a healthy tissue indicates a response to the cancer therapy with an ability to inhibit function and/or activity of PD-1, PD-L1 and/or PD-L2.

134. The method of any one of claims 125 to 133, wherein a lack of upregulation of pathway selected from Mapk8ip1, Trim7, Elk1, Lrg1, Arg1, Rap1, and Ras compared to another biological sample from patient that is known to be sensitive to anti-PD-1 therapy indicates a response to the cancer therapy with an ability to inhibit function and/or activity of PD-1, PD-L1 and/or PD-L2.

135. The method of any one of claims 125 to 134, wherein a lack of upregulation of pathway selected from Mapk8ip1, Trim7, Elk1, Lrg1, Arg1, Rap1, and Ras compared to a prior biological sample obtained from the subject indicates a development of lack of a response to the cancer therapy with an ability to inhibit function and/or activity of PD-1, PD-L1 and/or PD-L2.

136. The method of any one of claims 125 to 135, wherein the biological sample is a fresh tissue sample, frozen tumor tissue specimen, cultured cells, circulating tumor cells, or a formalin-fixed paraffin-embedded tumor tissue specimen.

137. The method of any one of claims 125 to 136, wherein the biological sample is a biopsy sample.

138. The method of claim 137, wherein the biopsy sample is selected from endoscopic biopsy, bone marrow biopsy, endoscopic biopsy (e.g., cystoscopy, bronchoscopy and colonoscopy), needle biopsy (e.g., fine-needle aspiration, core needle biopsy, vacuum-assisted biopsy, X-ray-assisted biopsy, computerized tomography (CT)-assisted biopsy, magnetic resonance imaging (MRI)-assisted biopsy and ultrasound-assisted biopsy), skin biopsy (e.g., shave biopsy, punch biopsy, and incisional biopsy) and surgical biopsy.

139. The method of any one of claims 125 to 138, wherein the biological sample comprises a body fluid selected from blood, plasma, serum, lacrimal fluid, tears, bone marrow, blood, blood cells, ascites, tissue or fine needle biopsy sample, cell-containing body fluid, free floating nucleic acids, sputum, saliva, urine, cerebrospinal fluid, peritoneal fluid, pleural fluid, feces, lymph, gynecological fluid, skin swab, vaginal swab, oral swab, nasal swab, washing or lavage such as a ductal lavage or broncheoalveolar lavage, aspirate, scraping, bone marrow specimen, tissue biopsy specimen, surgical specimen, feces, other body fluids, secretions, and/or excretions, and/or cells therefrom.

140. The method of any one of claims 125 to 139, wherein the biological sample is obtained by a technique selected from scrapes, swabs, and biopsy.

141. The method of claim 140, wherein the biological sample is obtained by use of brushes, (cotton) swabs, spatula, rinse/wash fluids, punch biopsy devices, puncture of cavities with needles or surgical instrumentation.

142. The method of any one of claims 136 to 141, wherein the biological sample comprises at least one tumor cell.

143. The method of claim 126, 127 or 142, wherein the tumor is selected from Hodgkin's and non-Hodgkin's lymphoma, B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; or chronic myeloblastic leukemia, basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (e.g., that associated with brain tumors), Meigs' syndrome cancer; renal carcinoma; colorectal cancer; and adrenal cancer.

144. The method of any one of claims 125 to 143, wherein the evaluating is performed by DNA sequencing, RNA sequencing, immunohistochemical staining, western blotting, in cell western, immunofluorescent staining, ELISA, and fluorescent activating cell sorting (FACS) or a combination thereof.

145. The method of any one of claims 125 to 144, wherein the evaluating is performed by contacting the sample with an agent that specifically binds to one or more proteins encoded by a pathway selected from Mapk8ip1, Trim7, Elk1, Lrg1, Arg1, Rap1, and Ras.

146. The method of claim 145, wherein the agent that specifically binds to one or proteins comprises an antibody, antibody-like molecule or binding a fragment thereof.

147. The method of any one of claims 125 to 146, wherein the evaluating is performed by contacting the sample with an agent that specifically binds to one or more of nucleic acids of one or more genes associated with a pathway selected from Mapk8ip1, Trim7, Elk1, Lrg1, Arg1, Rap1, and Ras.

148. The method of claim 147, wherein the agent that specifically binds to one or more of the nucleic acids is a nucleic acid primer or probe.

149. A method of treating a cancer in a subject in need thereof, the method comprising administering a cancer therapy is selected from: an antimetabolite chemotherapeutic, a topoisomerase inhibitor, a protein translation inhibitor, a ribosome biogenesis inhibitor, an inhibitor of rRNA and/or tRNA synthesis, an inhibitor of synthesis of amino acids, an inhibitor of uptake of amino acids, a modulator of post-translational modification, a modulator of protein degradation, a modulator of protein transport, a topoisomerase inhibitor, wherein
the subject has received or is receiving an anticancer treatment with an ability to inhibit function and/or activity of PD-1, PD-L1 and/or PD-L2;
wherein the subject has developed a lack of response, resistance or recalcitrance to the cancer therapy with an ability to inhibit function and/or activity of PD-1, PD-L1 and/or PD-L2; and
wherein pathway selected from Mapk8ip1, Trim7, Elk1, Lrg1, Arg1, Rap1, and Ras is upregulated in the at least one tumor cell of the subject compared to a healthy tissue, a prior biological sample obtained from the subject, or another biological sample from patient that is known to be sensitive to anti-PD-1 therapy.

150. A method of treating a cancer in a subject in need thereof, the method comprising:
(a) administering an anticancer treatment with an ability to inhibit function and/or activity of PD-1, PD-L1 and/or PD-L2;
(b) evaluating anti-tumor response with the anticancer treatment with an ability to inhibit function and/or activity of PD-1, PD-L1 and/or PD-L2 by monitoring a tumor size reduction in the subject;
(c) administering Trim7 modulator and/or a proteasome inhibitor if a lack of tumor size reduction is observed;
(d) re-evaluating anti-tumor response with the anticancer treatment with an ability to inhibit function and/or activity of PD-1, PD-L1 and/or PD-L2 by monitoring the tumor reduction in the subject; and
(e) withdrawing Trim7 modulator administration if a tumor size reduction is observed.

151. A method of treating a cancer in a subject in need thereof, the method comprising:
(a) administering an anticancer treatment with an ability to inhibit function and/or activity of PD-1, PD-L1 and/or PD-L2;
(b) evaluating anti-tumor response with the anticancer treatment with an ability to inhibit function and/or activity of PD-1, PD-L1 and/or PD-L2 using the steps of:
(i) obtaining a biological sample from the subject;
(ii) evaluating the biological sample for the overexpression and/or activation of TRIM7;
(c) administering Trim7 modulator and/or a proteasome inhibitor if an overexpression and/or activation of TRIM7 is observed;
(d) re-evaluating anti-tumor response with the anticancer treatment with an ability to inhibit function and/or activity of PD-1, PD-L1 and/or PD-L2 using the steps of:
(i) obtaining a biological sample from the subject;
(ii) evaluating the biological sample for the overexpression and/or activation of TRIM7; and
(e) withdrawing Trim7 modulator administration if an overexpression and/or activation of TRIM7 is not observed.

152. A method of treating a cancer in a subject in need thereof, the method comprising:
(a) administering an anticancer treatment with an ability to inhibit function and/or activity of PD-1, PD-L1 and/or PD-L2;
(b) evaluating overexpression and/or activation of TRIM7 using the steps of:
(i) obtaining a biological sample from the subject; and
(ii) evaluating the biological sample for the overexpression and/or activation of TRIM7;
(c) administering Trim7 modulator and/or a proteasome inhibitor if an overexpression and/or activation of TRIM7 is observed;
(d) re-evaluating overexpression and/or activation of TRIM7 using the steps of:
(i) obtaining a biological sample from the subject;
(ii) evaluating the biological sample for the overexpression and/or activation of TRIM7; and
(e) withdrawing Trim7 modulator administration if an overexpression and/or activation of TRIM7 is not observed.

153. The method of claim one of claims 150 to 152, wherein the Trim7 modulator is a Trim 7 inhibitor.

154. The method of any one of claims 150 to 153, wherein the Trim7 modulator is selected a small interference RNA (siRNA), a short hairpin RNA (shRNA), a microRNA (miRNA), an antisense RNA, a guide RNA (gRNA), a small molecule, an antibody, a peptide, and a peptidomimetic.

155. The method of claim 154, wherein the small interference RNA (siRNA), the short hairpin RNA (shRNA), the microRNA (miRNA), the antisense RNA, or the guide RNA (gRNA) inhibit the production of Trim7 protein.

156. The method of claim 154, wherein the peptidomimetic mimics a target of Trim7 and thereby inhibits the activity of Trim7.

157. The method of claim 153, wherein the Trim 7 inhibitor is a small molecule or peptide inhibitor that binds Trim7 protein at or near protein segments selected from MAAVGPRTGPGTGAEALALAAEL (SEQ ID NO: 1), AATRAPPFPLPCP (SEQ ID NO: 2), HGSQAAAARAAAARCG (SEQ ID NO: 3) and NVSLKTFVLKGMLKKFKEDLRGELEKEEKV (SEQ ID NO: 4).

158. The method of claim one of claims 150 to 152, wherein the Trim7 modulator is an mitogen- and stress-activated kinase 1 (MSK1) inhibitor, wherein the MSK1 inhibitor modulates Trim7 via downstream effect of an inhibition of MSK1.

159. The method of claim 158, wherein the MSK1 inhibitor is selected from Ro 31-8220, SB-747651A, and H89.

160. The method of claim 159, wherein the MSK1 inhibitor is SB-747651A.

161. The method of any one of claims 150 to 160, wherein the proteasome inhibitor is selected from bortezomib, carfilzomib, ixazomib, oprozomib, delanzomib and marizomib.

162. The method of any one of claims 151 to 161, wherein the evaluating is performed by DNA sequencing, RNA sequencing, immunohistochemical staining, western blotting, in cell western, immunofluorescent staining, ELISA, and fluorescent activating cell sorting (FACS) or a combination thereof.

163. The method of any one of claims 151 to 162, wherein the evaluating is performed by contacting the sample with an agent that specifically binds to one or more proteins encoded by the Trim7 pathway.

164. The method of claim 163, wherein the agent that specifically binds to one or proteins comprises an antibody, antibody-like molecule or binding a fragment thereof.

165. The method of any one of claims 151 to 164, wherein the evaluating is performed by contacting the sample with an agent that specifically binds to one or more of nucleic acids of one or more genes associated with the Trim7 pathway.

166. The method of claim 165, wherein the agent that specifically binds to one or more of the nucleic acids is a nucleic acid primer or probe.

167. The method of any one of claims 151 to 166, wherein the evaluating is performed by assaying a E3 ubiquitin ligase activity.

168. The method of any one of claims 151 to 167, wherein the evaluating is performed by assaying protein ubiquitylation and/or K48-linked ubiquitylation of stimulator of interferon genes (STING) and/or AP-1 co-activator RACO-1.

169. The method of any one of claims 151 to 168, wherein the evaluating is performed by assaying c-Jun/AP1 activation via Ras-Raf-MEK-ERK signaling and/or an increase in AP1 mediated gene expression.

170. The method of any one of claims 151 to 169, wherein the evaluating is performed by assaying ubiquitination and stabilization of AP1 co-activator RACO-1.

171. The method of any one of claims 151 to 170, wherein the evaluating is performed by assaying K63-linked ubiquitylation of target proteins, including proteins involved with cell proliferation and innate immune responses.

172. The method of any one of claims 151 to 171, wherein the evaluating is performed by assaying Trim7 phosphorylation, K63-linked ubiquitylation and/or protein level of the AP-1 co-activator RACO-1.

173. The method of any one of claims 151 to 172, wherein the evaluating is performed by assaying the upregulation of IFNβ, IP-10 and/or Rantes.
